(19) United States
(12) Patent Application Publication
(10) Pub. No.: US 2026/0053853 A1
(43) Pub. Date: Feb. 26, 2026

(54) ENGINEERED T CELL RECEPTOR CONSTRUCTS FOR CANCER THERAPY

(71) Applicants: HADASIT MEDICAL RESEARCH SERVICES AND DEVELOPMENT LTD., Jerusalem (IL); BAR-ILAN UNIVERSITY, Ramat Gan (IL)

(72) Inventors: Michal LOTEM, Jerusalem (IL); Shira KLEIN SILBERMAN, Jerusalem (IL); Galit EISENBERG, Jerusalem (IL); Inna BEN DAVID, Jerusalem (IL); Anna KUZNETS, Jerusalem (IL); Yael GELFAND, Jerusalem (IL); Cyrille Joseph COHEN, Jerusalem (IL); Astar SHAMUL, Jerusalem (IL)

(21) Appl. No.: 18/995,761

(22) PCT Filed: Jul. 27, 2023
(86) PCT No.: PCT/IL2023/050781

§ 371 Date: Jan. 16, 2025

Related U.S. Application Data
(60) Provisional application No. 63/392,876, filed on Jul. 28, 2022.

(51) Int. Cl. A61K 35/17; A61K 40/11; A61K 40/32; A61K 40/42; A61P 35/00; C07K 14/725; C07K 16/30; C12N 5/00; C12N 5/0783; C12N 15/86

(52) U.S. Cl. CPC A61K 35/17; A61K 40/11; A61K 40/32; A61K 40/4269; A61P 35/00; C07K 14/7051; C07K 16/3069; C12N 5/0018; C12N 5/0636; C12N 15/86; C07K 2317/52; C07K 2317/565; C07K 2319/02; C12N 2501/2302; C12N 2501/51; C12N 2501/515; C12N 2510/00; C12N 2740/10043

(57) ABSTRACT

The invention relates to engineered T cell receptors (TCRs) directed to cancer testis antigen New York Esophageal Squamous Cell Carcinoma-1 (NY-ESO-1), useful in the treatment of cancer. In particular, provided are isolated TCR polypeptides, as well as corresponding nucleic acid molecules and cell compositions, characterized by improved properties that are particularly adapted to cancer immunotherapy.

### FIELD OF THE INVENTION

The invention relates to engineered T cell receptors directed to NY-ESO-1, nucleic acid constructs encoding them, and use thereof in cancer therapy.

### BACKGROUND OF THE INVENTION

Cancer is a leading cause of death worldwide, accounting for nearly ten million deaths in 2020, or nearly one in six deaths. Conventional cancer therapies such as chemotherapeutic drugs and radiotherapy typically have low specificity and can act on both healthy and diseased tissues, generating severe side effects. This low specificity laid the ground for the development of targeted-therapy approaches, including various immunotherapeutic modalities. Among the main approaches employed in immunotherapy are monoclonals antibodies, immune system modulators (e.g. cytokines), therapeutic vaccines, immune checkpoint inhibitors and adoptive cell transfer (ACT).

One of the major challenges regarding cancer therapy is the immune-evasion ability of tumors. Cancer cells exploit the schemes that the immune system uses to control the extent of immune reactivity such as upregulated expression of checkpoint proteins, activation of cell death programs, and accumulation of various immunosuppressive cells to escape the immune attack, and may employ additional immune tolerance mechanisms.

ACT has been appreciated as a promising treatment for patients with cancer. Utilization of ACT can enhance the function of the immune system or improve the specificity and persistence of immune cells transferred to the patient. Cancer ACT typically utilizes immune cells isolated from the blood of the patient (or from a suitable donor), which may undergo ex-vivo cancer specificity selection and/or genetic modifications arming these cells with anti-tumor functions. Subsequently, an expansion step of the selected or modified cells to large numbers, followed by infusion of the expanded cells back to the patient to fight the cancer, are typically performed.

ACT includes a number of different types of immunotherapy protocols, one of which involves T-cell receptor (TCR) transduced T cells (TCR-T), in which immune cells are genetically modified or manipulated to express a TCR with a desired tumor antigen specificity prior to administration into a subject. Although promising, some setbacks were encountered while developing TCR-T compositions for ACT, e.g. with respect to the expression and/or function of the transfected TCRs. For example, assembly of mispaired endogenous and transfected TCRs was reported to produce TCRs with unexpected specificity, including self-reactive TCRs. It has been suggested that T cell recognition of targeted antigens on normal tissues, or of unrelated antigens caused by TCR cross-reactivity, are among the main challenges in developing TCR-T modalities with adequate efficacy and safety. Additional setbacks relate e.g. to insufficient expression, stability and/or functionality in the engineered cells, which may subsequently be associated with inadequate functional avidity. In addition, exhausted effectors generated by overstimulation and tonic signaling may further impair the therapeutic outcome.

Attempts to modify various positions and structural elements in the sequences of the TCR chains aiming to improve their functional properties have been reported. The development of modified engineered TCRs is discussed e.g. in Bialer G. et al (J. Immunol. 2010, 184: 6232-6241, Cohen C. J. et al (Cancer Res. 2006 Sep. 1; 66(17): 8878-8886), Johnson L. A. et al (J Immunol. 2006 Nov. 1; 177(9): 6548-6559), Daniel-Meshulam I. et al (Front. Immunol., 6 Jul. 2012), Haga-Friedman A. et al (J. Immunol. 2012, 188: 5538-5546), Cohen C. J. et al (Cancer Res. 2007, April 15; 67(8): 3898-3903), Kuball J. et al. (Blood. 2007, 109:2331-2338) and Ikeda H (International Immunology, Vol. 28, No. 7, pp. 349-353). To date, preclinical and clinical studies have demonstrated various levels of feasibility, safety, and efficacy using TCR-engineered T cells to treat cancer and viral infections, and there is still a need for developing TCRs with improved expression and function compatible with ACT protocols.

Cancer related epitopes exclusively expressed on tumor cells are vigorously sought out for TCR-T in order to reduce bystander cytotoxic effects in healthy tissues of cancer patients. NY-ESO-1 or New York esophageal squamous cell carcinoma 1 is a well-known cancer-testis antigen with re-expression in various cancer types. NY-ESO-1 expression has been reported in a wide range of tumor types, including neuroblastoma, myeloma, metastatic melanoma, synovial sarcoma, bladder cancer, esophageal cancer, hepatocellular cancer, head and neck cancer, non-small cell lung cancer, ovarian cancer, prostate cancer, and breast cancer. Its ability to elicit spontaneous humoral and cellular immune responses, together with its restricted expression pattern, have rendered it a good candidate target for cancer immunotherapy (Thomas R, et al. Front Immunol. 2018, 9:947). Reactive epitopes of human NY-ESO-1 were identified as amino acids 157-167.

Robbins et al. (J. Immunol. 2008, 180: 6116-6131) discloses the generation of single and dual amino acid substitution variants in the TCR CDRs of three TCRs that recognize tumor-associated antigens. Among the variants created are variants of the 1G4 TCR, that recognizes a peptide corresponding to amino acid residues 157-165 of the human cancer testis Ag NY-ESO-1 in the context of the HLA-A*02 class I allele.

EP2016102, to some of the present inventors and co-workers, is directed to chimeric TCRs comprising a variable region of a human TCR and a constant region comprising at least an extracellular domain of a constant region of a non-human TCR, as well as functional variants thereof. The disclosed chimeric TCR may have antigenic specificity for a cancer antigen selected from the group consisting of MART-I, gp-100, p53, and NY-ESO-1.

Additional TCRs directed to inter alia NY-ESO-1 are disclosed for example in U.S. Ser. No. 10/201,597, WO2020188348, US20210163891, US20190100592, US20210317184, WO2008037943, US 20200040358 and U.S. Pat. No. 8,088,379.

A number of clinical trials examining NY-ESO-1-specific engineered TCR-T have been initiated. For example, Robbins, et al, (Clin Cancer Res. 2015 Mar. 1; 21(5): 1019-1027), Rapoport A. P. et al, (Nat Med. 2015, August; 21(8): 914-921), Stadtmauer E A, et al, (Blood Adv, 2019, 3(13), 2022), Ramachandran I, et al, (J Immunother Cancer. 2019, 7:276) and D'Angelo S. P. et al, (Cancer Discov. 2018, 8(8): 944-957) report on clinical studies involving NY-ESO-1-directed immunotherapy. Some of the studies suggested impaired efficacy and/or safety. For example, while the treatments were found to be effective in some patients, they often resulted in lack of response (or of complete response) and/or short duration of responses in the majority of the tested patients (e.g., due to poor persistence of the adoptively transferred cells). Among the adverse effects reported were immune deficiencies (thrombocytopenia, lymphopenia, leukopenia, neutropenia, anemia), gastrointestinal symptoms (diarrhea, decreased appetite, nausea), fatigue, hypoxia, infection, hypophosphatemia, cytokines release syndrome, autologous graft versus host disease (GVHD), immune effector cells associated neurologic toxicity (ICANS) and treatment-related death.

Hence, despite reported success in preclinical trials and in certain groups of cancer patients, many TCR-T modalities have demonstrated impaired or insufficient therapeutic activity (or otherwise applicability) in clinical practice. To date, there are no engineered TCR-T therapy products with regulatory approval for marketing.

Thus, there remains a need for additional improved means, methods, and compositions for NY-ESO-1 TCR-based cancer ACT. In particular, the development of NY-ESO-1 TCR-based therapeutic modalities for ACT, providing enhanced efficacy and/or safety, would be highly beneficial.

### SUMMARY OF THE INVENTION

The invention relates to engineered T cell receptors (TCRs) directed to cancer testis antigen New York Esophageal Squamous Cell Carcinoma-1 (NY-ESO-1), to nucleic acid constructs encoding them, and to their use in cancer therapy. In particular, provided are isolated TCR polypeptides, as well as corresponding nucleic acid molecules and cell compositions, characterized by improved properties that are particularly adapted to cancer immunotherapy.

The invention is based, in part, on the development of improved constructs for providing NY-ESO-1-specific lymphocyte compositions amenable for cancer immunotherapy. As disclosed herein, a nucleic acid construct, encoding a novel engineered NY-ESO-1-specific TCR, was developed. The construct, cloned into a retroviral vector comprising long terminal repeats (LTRs) from the murine stem cell virus (MSCV), was characterized in a variety of in vitro and in vivo assays. Remarkably, transduction of human primary lymphocytes with the vector resulted in a significantly enhanced anti-tumor response, not only as compared to lymphocytes transduced with a non-relevant TCR, but also as compared to those transduced with a hitherto-known TCR directed to the same epitope of NY-ESO-1. Further, a clinical-grade pharmaceutical composition comprising the construct, manufactured in accordance with GMP requirements, was found to be highly effective and safe in the treatment of tumors in vivo.

Accordingly, disclosed herein is the development and preparation of improved therapeutic modalities for cancer immunotherapy, including nucleic acid-based pharmaceutical compositions and cell compositions. In various embodiments, compositions in accordance with the principles of the invention may be used to overcome or diminish persistent deficiencies typically associated with adoptive transfer therapies.

Thus, the invention in embodiments thereof relates to an isolated TCR directed to NY-ESO-1. As disclosed herein, isolated TCRs (also referred to herein as isolated or engineered TCR complexes, molecules or polypeptides) bind specifically to an HLA-A2-presented epitope comprising the amino acid sequence SLLMWITQC (SEQ ID NO: 21), corresponding to residues 157 to 165 of NY-ESO-1. The TCRs are characterized by a unique combination of structural elements as detailed below, providing for advantageous functional properties. In some embodiments, TCRs in accordance with the invention comprise complementarity determining region (CDR) sequences as set forth in Table 1 below. Additional structural and functional properties characteristic of TCRs in accordance with embodiments of the invention are further detailed below.

In one aspect of the present invention, there is provided an isolated TCR directed to NY-ESO-1, comprising:
(a) a TCR α chain comprising:
   i. a variable region (VR) comprising a CDR1 having the amino acid sequence of SEQ ID NO: 1, a CDR2 having the amino acid sequence of SEQ ID NO: 2, and a CDR3 having the amino acid sequence of SEQ ID NO: 3;
   ii. a constant region (CR) comprising a cysteine residue at position 47 thereof and the amino acid sequence as set forth in SEQ ID NO: 22 at positions 250-254 thereof;
(b) a TCR β chain comprising:
   i. a VR comprising a CDR1 having the amino acid sequence of SEQ ID NO: 4, a CDR2 having the amino acid sequence of SEQ ID NO: 5, and a CDR3 having the amino acid sequence of SEQ ID NO: 6; and
   ii. a CR comprising a cysteine residue at position 57 thereof; and
(c) a plurality of interchain disulfide bonds between the α chain and β chain.

In one embodiment, the TCR comprises a first disulfide bond between the cysteine residues at positions 94 of the α chain CR and 131 of the β chain CR, and a second disulfide bond between the cysteine residues at positions 47 of the α chain CR and 57 of the β chain CR. In another embodiment, the transmembrane region of the α chain has the amino acid sequence LLVIVLRILLLKVAGFNLLMT (SEQ ID NO: 23). In a further embodiment, the TCR comprises a TCR α chain having the amino acid sequence as set forth in SEQ ID NO: 17, optionally excluding the signal peptide at positions 1-20 thereof. In yet another embodiment the TCR comprises a TCR β chain having the amino acid sequence as set forth in SEQ ID NO: 18, optionally excluding the signal peptide at positions 1-21 thereof. In another embodiment, each of the α and β chains exhibits at least 95% sequence identity to SEQ ID NOs: 17 and 18, respectively, and said TCR is capable of specific binding to an HLA-A2-presented epitope comprising the amino acid sequence of SEQ ID NO: 21. In yet another embodiment, each of the α and β chains exhibits at least 95% sequence identity to SEQ ID NOs: 17 and 18, respectively, and said TCR is capable of specific binding to an HLA-A2-presented epitope comprising the amino acid sequence of SEQ ID NO: 21 with high functional avidity.

In another aspect, there is provided a nucleic acid construct encoding the isolated TCR. In another embodiment, the nucleic acid construct comprises the nucleic acid sequences as set forth in SEQ ID NOs: 7-9, encoding the CDR1, CDR2 and CDR3 of the α chain, respectively, and the nucleic acid sequences as set forth in SEQ ID NOs: 10-12, encoding the CDR1, CDR2 and CDR3 of the β chain, respectively. In a further embodiment, the nucleic acid construct comprises a first nucleic acid molecule encoding the α chain and having the nucleic acid sequence as set forth in SEQ ID NO: 19, and a second nucleic acid molecule encoding the β chain and having the nucleic acid sequence as set forth in SEQ ID NO: 20. In another embodiment, said first and second nucleic acid molecules are operatively linked to one or more transcription control elements. In yet another embodiment, the one or more transcription control elements are capable of inducing or enhancing the expression of said TCR chains in a human lymphocyte (in particular in T cells).

In another embodiment, the nucleic acid construct comprises a first nucleic acid molecule encoding the α chain and having at least 95% identity to the nucleic acid sequence as set forth in SEQ ID NO: 19, and a second nucleic acid molecule encoding the β chain and having at least 95% identity to the nucleic acid sequence as set forth in SEQ ID NO: 20, wherein the encoded TCR is capable of specific binding to an HLA-A2-presented epitope comprising the amino acid sequence of SEQ ID NO: 21. In yet another embodiment, the nucleic acid construct comprises a first nucleic acid molecule encoding the α chain and having at least 95% identity to the nucleic acid sequence as set forth in SEQ ID NO: 19, and a second nucleic acid molecule encoding the β chain and having at least 95% identity to the nucleic acid sequence as set forth in SEQ ID NO: 20, wherein the encoded TCR is capable of specific binding to an HLA-A2-presented epitope comprising the amino acid sequence of SEQ ID NO: 21 with high functional avidity. In yet another embodiment, the encoded TCR is capable of exerting tumor-specific reactivity in a CD8-independent manner. In yet another embodiment, the nucleic acid construct is capable of providing an enhanced level and/or duration of expression of the encoded TCR, as compared to a construct encoding a naturally-occurring TCR directed to an HLA-A2-presented epitope as set forth in SEQ ID NO: 21. In yet another embodiment, the first nucleic acid molecule and the second nucleic acid molecule are connected by a third nucleic acid molecule encoding a linker. In yet another embodiment, the linker is a 2A peptide.

In another aspect, there is provided a nucleic acid construct comprising at least one nucleic acid molecule selected from the group consisting of SEQ ID NOs: 19 and 20. In another aspect, there is provided an expression vector comprising a nucleic acid construct as disclosed herein. In another embodiment, the invention provides a host cell comprising the vector. In a further embodiment, there is provided a pharmaceutical composition comprising a therapeutically effective amount of the vector.

In another aspect, there is provided a cell composition for cancer immunotherapy, comprising a therapeutically effective amount of immune cells (typically and preferably a T-cell containing population) engineered to express a TCR directed to NY-ESO-1, the TCR comprising:
(a) a TCR α chain comprising:
   (i) a VR comprising a CDR1 having the amino acid sequence of SEQ ID NO: 1, a CDR2 having the amino acid sequence of SEQ ID NO: 2, and a CDR3 having the amino acid sequence of SEQ ID NO: 3;
   (ii) a CR comprising a cysteine residue at position 47 thereof and the amino acid sequence as set forth in SEQ ID NO: 22 at positions 250-254 thereof;
(b) a TCR β chain comprising:
   (i) a VR comprising a CDR1 having the amino acid sequence of SEQ ID NO: 4, a CDR2 having the amino acid sequence of SEQ ID NO: 5, and a CDR3 having the amino acid sequence of SEQ ID NO: 6; and
   (ii) a CR comprising a cysteine residue at position 57 thereof; and
(c) a plurality of interchain disulfide bonds between said α chain and β chain.

In one embodiment, the cell composition is adapted for adoptive transfer therapy (ACT). In another embodiment, the immune cells are a T cell-containing cell population. In a particular embodiment, the immune cells are a T cell-containing cell population and said composition is adapted for ACT. In another embodiment, at least 60%, 70%, 80% or 90% of the immune cells are characterized by surface expression of the engineered TCR (eTCR⁺ cells), wherein each possibility represents a separate embodiment of the invention. In another embodiment, at least 70% of the immune cells are CD3⁺ eTCR⁺ cells and comprise both CD4⁺ and CD8⁺ cells. In yet another embodiment, the immune cells were engineered to express the TCR by administration of a nucleic acid construct encoding said TCR, and the cell composition is characterized by an average copy number of five or less (e.g. 1, 2, 3, 4 or 5) construct molecules per cell. In yet another embodiment, the TCR comprises a TCR α chain having the amino acid sequence as set forth in SEQ ID NO: 17, optionally excluding the signal peptide at positions 1-20 thereof, and a TCR β chain having the amino acid sequence as set forth in SEQ ID NO: 18, optionally excluding the signal peptide at positions 1-21 thereof. In yet another embodiment, the nucleic acid construct comprises a first nucleic acid molecule encoding the α chain and having the nucleic acid sequence as set forth in SEQ ID NO: 19, and a second nucleic acid molecule encoding the β chain and having the nucleic acid sequence as set forth in SEQ ID NO: 20.

In another embodiment, the cell composition is prepared by a process comprising:
a. provided a T-cell containing cell population,
b. culturing the cell population in the presence of a CD3-specific antibody and interleukin-2 (IL-2),
c. engineering the cells resulting from step b. to express said TCR,
d. expanding the cells resulting from step c. so as to obtain a therapeutically effective amount of expanded engineered cells, and
e. harvesting the expanded engineered cells resulting from step d.

In another embodiment, the T-cell containing cell population provided in step a. is a peripheral blood mononuclear cells (PBMC) population. In another embodiment, step b. further comprises culturing said cells with a CD28-specific antibody, and the CD3-specific antibody and the CD28-specific antibody are surface-bound. In another embodiment, step d. is performed by incubating the cells following step c. with IL-2 and optionally feeder cells. In a particular embodiment. step d. is performed by incubating the cells following step c. with IL-2 and feeder cells. In another embodiment, step d. is performed in the absence of supplementation of antibodies. In another embodiment, step d. is performed so as to obtain at least 10⁹ CD3⁺ eTCR⁺ cells. In another embodiment, step e. is performed within 7-21 days and typically within 14-21 days of initiating step b.

In another aspect, there is provided a process for preparing a cell composition for ACT, the process comprising:
a. provided a T-cell containing cell population,
b. culturing the cells in the presence of a CD3-specific antibody and IL-2,
c. engineering the cells resulting from step b. to express a TCR as disclosed herein,
d. expanding the cells resulting from step c. so as to obtain a therapeutically effective amount of expanded engineered cells, and
e. harvesting the expanded engineered cells resulting from step d.

In one embodiment, the process comprises:
a. provided a PBMC population,
b. culturing the cell population in the presence of a CD3-specific antibody and IL-2,
c. engineering the cells to express the TCR by incubating said cells with a viral vector comprising a nucleic acid construct encoding said TCR,
d. expanding the cells following step c. in the presence of IL-2 and feeder cells, and in the absence of supplementation of antibodies, so as to obtain at least 10⁹ CD3⁺ cells expressing said TCR, and
e. harvesting the expanded cells within 14-15 days of initiating step b.

In another embodiment, the resulting cell composition comprises at least 70% CD3⁺ eTCR⁺ cells which include both CD4⁺ and CD8⁺ cells. In another embodiment, step b. comprises administering to the cells an expression vector as disclosed herein (encoding a TCR of the invention).

In another aspect of the present invention, there is provided a method of treating cancer in a subject in need thereof, comprising administering to the subject the cell composition as disclosed herein. In one embodiment, the cells of the composition (resulting from step e.) are autologous to the subject.

In another aspect, there is provided a method of treating cancer in a subject in need thereof, comprising administering to the subject the pharmaceutical composition as disclosed herein.

In another aspect, there is provided a method of enhancing anti-tumor immunity in a subject in need thereof, comprising administering to the subject the cell composition or the pharmaceutical composition as disclosed herein. Each possibility represents a separate embodiment of the invention.

In some embodiments of the methods of the invention, the subject is afflicted with a NY-ESO-1 expressing tumor. In another embodiment, the subject is HLA-A2-positive. In another embodiment, the tumor is selected from the group consisting of: melanoma, bladder, ovarian, lung, breast, and prostate tumors. In another embodiment, the tumor is selected from the group consisting of: melanoma, myeloma, bladder, ovarian, lung, breast, synovial and prostate tumors. Each possibility represents a separate embodiment of the invention.

In another aspect, the invention relates to a cell composition of the invention for use in treating cancer in a subject in need thereof. In another embodiment the subject is afflicted with a NY-ESO-1 expressing tumor, and/or said subject is HLA-A2-positive. In another embodiment the tumor is selected from the group consisting of: melanoma, myeloma, bladder, ovarian, lung, breast, synovial and prostate tumors. In another embodiment the cells of the composition are autologous to the subject.

In another aspect, there is provided a pharmaceutical composition comprising a therapeutically effective amount of a vector of the invention, for use in treating cancer in a subject in need thereof.

In another embodiment the subject is afflicted with a NY-ESO-1 expressing tumor, and/or said subject is HLA-A2-positive. In another embodiment the tumor is selected from the group consisting of: melanoma, myeloma, bladder, ovarian, lung, breast, synovial and prostate tumors.

In another aspect, there is provided a cell composition of the invention, for use in enhancing anti-tumor immunity in a subject in need thereof. In another embodiment the subject is afflicted with a NY-ESO-1 expressing tumor, and/or said subject is HLA-A2-positive. In another embodiment the tumor is selected from the group consisting of: melanoma, myeloma, bladder, ovarian, lung, breast, synovial and prostate tumors. In another embodiment the cells of the composition are autologous to the subject.

In another aspect, there is provided a pharmaceutical composition comprising a therapeutically effective amount of a vector of the invention, for use in enhancing anti-tumor immunity in a subject in need thereof. In another embodiment the subject is afflicted with a NY-ESO-1 expressing tumor, and/or said subject is HLA-A2-positive. In another embodiment the tumor is selected from the group consisting of: melanoma, myeloma, bladder, ovarian, lung, breast, synovial and prostate tumors.

Other objects, features and advantages of the present invention will become clear from the following description and drawings.

### BRIEF DESCRIPTION OF THE DRAWINGS

FIGS. 1A-1C depict cytokine secretion levels upon co-culture of eTCR, 1G4, or NGFR-transduced cells with cognate target cells (NY-ESO-1-expressing melanoma lines A375 and 624.38) or with control melanoma cells (888A2). Tumor necrosis factor (TNFα), interleukin 2 (IL2) and interferon γ (IFN-γ) levels in the co-culture supernatant were determined by ELISA. FIG. 1A—TNFα concentrations. FIG. 1B—IL2 concentrations. FIG. 1C—IFN-γ concentrations.

FIG. 2 depicts expression levels of the T cell activation marker, OX40, following the co-culture of eTCR, 1G4, or NGFR-transduced cells with A375, and 624.38 (“624”) target cells and 888A2 melanoma cell line as control. The transduced cells were stained with anti-OX40 antibodies and measured by fluorescence activated cell sorting (FACS). Results are normalized to the activity of the NGFR-transduced cells against A375 cells.

FIG. 3 depicts the expression levels of the T cell activation marker, 4-1BB, in CD8⁺ cells, following the co-culture of cells transduced with either eTCR, 1G4, or NGFR, with A375 or 624.38 target cells. The transduced cells were stained with differentially labeled anti-4-1BB and anti-CD8 antibodies and measured by FACS.

FIG. 4 depicts the expression level of the eTCR in PG13 cells corresponding to initial 12 clones of eTCR. PG13 cells transduced only with medium were used as negative control and appear as PG13. The cells were stained with a PE-conjugated anti-Vβ13 antibody directed to the specific beta chain of the eTCR and evaluated by FACS.

FIG. 5 illustrates target-induced IFN-γ secretion levels from PBMC activated and transduced with different dilutions (1:1, 1:2, 1:4, 1:8) of eTCR subclones 1.2 and 1.16. IFN-γ concentration in the supernatants following co-culture with melanoma A375 cells was determined by ELISA. Undiluted clone 1.3 served as a positive control and PG13 cells transduced with only medium (“no vector”) served as a negative control.

FIG. 6. depicts FACS plots of caspase-3 levels in A375 cells (expressing NY-ESO-1) following co-culture with PBMCs activated and transduced with eTCR subclones. Cells were stained with a cell tracer marker DDAO and for caspase-3 as an indicator of apoptosis. Cells were examined for percentage of caspase-3 and DDAO expression by FACS.

FIGS. 7A-7B. depict PCR results for the presence of replication-competent retrovirus (RCR) in 6 random samples of PBMC activated and transduced with eTCR vector. PCR was conducted on day 20 post transduction. FIG. 7A, PCR using primers for GALV (the viral envelope protein) as an indicator of RCR. FIG. 7B, PCR using primers for beta-actin (PAct) as a positive control.

FIG. 8 depicts the specific subsets and phenotypes of the cell composition manufactured under different expansion conditions. CD3⁺ T cells obtained at day 20 post transduction from the different expansion protocols were stained with antibodies directed to CD45RA and CCR7 and analyzed by flow cytometry. Lymphocytes transduced with a vector-free supernatant served as a negative control (“no vector”), lymphocytes transduced with the cell composition appear as “eTCR”. The tested expansion protocols: anti-CD3 and feeder cells appears as “aCD3”, Transact (Milteny) protocol appears as “Trans” and DynaBeads (Thermo Scientific) appears as “Dyna”. Effector memory cells lacking expression of both CCR7 and CD45RA appear as Tem (CD45RA⁻CCR7⁻, gray bars), and Temra cells express CD45RA but lack expression of CCR7 appear as T_{EMRA} (CD45RA⁺CCR7⁻, black bars). Results are presented as the percentile of each group (T_{EM} or T_{EMRA}) out of the total number of CD3⁺ cells.

FIG. 9 depicts results of a second analysis of the specific subsets and phenotypes of the cell composition manufactured under different expansion protocols as described in FIG. 8.

FIG. 10 depicts the proportion of different subsets of immune cells in the various cell compositions. Cells were stained for CD3, CD4, CD8, CD14, CD19, CD83 and CD56 and analyzed by flow cytometry. The results correspond to day 13 post transduction of experiment 2. The different treatment groups are as described in FIG. 8.

FIG. 11 depicts the proportion of different subsets of immune cells in the various cell compositions as describe in FIG. 10. The results correspond to day 20 post transduction of the same experiment as in FIG. 10.

FIG. 12 depicts the tumor volume at different time points post ACT in vivo. ACT was performed in cohorts of NSG mice inoculated with A375 melanoma cells and treated with cells transduced with eTCR vector (black line), cells transduced with negative control PG13 supernatants (no vector, dashed line), or with saline (no ACT, dotted line). Tumor volumes were measured at day 7, 10, 15, 18, 21 post ACT and are presented as mean t SD.

FIG. 13 depicts tumors weights obtained from ACT treated mice at day 22 post tumor inoculation. Tumors from mice treated with eTCR, no vector or saline as described in FIG. 12, were obtained and weighed, mean t SD are indicated.

FIGS. 14A-14F depict tumor volumes in individual A375 melanoma tumor bearing mice according to different ACT tested groups over time. The tested groups were treated with escalating doses of eTCR⁺ cells as ACT. Each line represents an individual mouse. FIG. 14A, control group of mice which received no treatment. FIG. 14B, control group of mice which received non-transduced cells. FIG. 14C, mice which received eTCR⁺ ACT dosage of 14×10⁶ cells/mouse. FIG. 14D, eTCR⁺ ACT dosage of 21×10⁶. cells/mouse. FIG. 14E, eTCR⁺ ACT dosage of 28×10⁶ cells/mouse. FIG. 14F, eTCR⁺ ACT dosage of 56×10⁶ cells/mouse.

FIG. 15 depicts Kaplan Meier curves of survival over time of A375 melanoma tumor bearing mice post ACT. The survival curves were calculated for each tested group (p<0.01): no treatment appears as tumor only—full diamond, negative control (no vector cells)—line with vertical dashes on the top, eTCR⁺ cells: 14×10⁶—full squares, 21×10⁶—full hexagon, 28×10⁶—full circle, 56×10⁶ empty triangle.

FIG. 16 depicts a photographic imaging of time-dependent bioluminescence of luciferase-expressing A375 melanoma cells, in post-ACT individual mice according to the tested groups as described in FIG. 14. The bioluminescence was monitored at day 6, 13, 20, 27 and 34 post tumor inoculation.

FIGS. 17A-17B depict flow cytometry plots corresponding to the persistence of eTCR⁺ cells in blood of NSG tumor bearing mice post ACT. Blood samples, obtained at day 33 following the first infusion of the ACT from mice administered with 56×10⁶ eTCR⁺ cells and achieved complete anti-tumor response, were stained and analyzed by flow cytometry. FIG. 17A —blood samples stained with FITC-conjugated anti human CD8 antibodies. FIG. 17B—the CD8⁺ cell population from FIG. 17A stained with PE-conjugated anti-Vβ13 antibodies.

FIG. 18 depicts tumor volume over time in female mice bearing luciferase-expressing A375 melanoma cells administered with eTCR⁺ cell compositions produced by different protocols. The protocols are: No treatment “tumor only”—dotted line with full circle; no vector—dashed line with full triangle; Dynabeads (13×10⁶ positive cells/17.3×10⁶ total) appears as DYna—black line with full squares; Transact (13×10⁶ positive cells/18.6×10⁶ total) appears as Trans—black line and dashes; and anti-CD3 with feeder cells (13×10⁶ positive cells/28×10⁶ total) appears as aCD3—black line with full circles.

FIG. 19 depicts Kaplan-Meier curves of survivals of the ACT treated mice as described in FIG. 18. No treatment appears “tumor only”—dashed line, no vector—dotted line, Dynabeads appears as DYna—thick line, Transact appears as Trans—thin line, anti-CD3 and feeder cells appears as Feeders—full squares.

FIG. 20 depicts target-specific TNFα secretion from cells expressing eTCR or the hitherto known TCR, 1G4. TNFα concentrations were measured following the co-culture of eTCR- or 1G4-transduced cells with T2 cells pulsed with different concentrations of NY-ESO-1 peptide (or the MART-1 peptide as a control.

FIG. 21 illustrates target-induced IFN-γ secretion levels from cell compositions prepared from PBMC originally isolated from a patient afflicted with uveal melanoma, transduced or not transduced with eTCR. Following incubation with T2 cells loaded or unloaded with NY-ESO-1 peptide at a 1:1 effector to target (E:T) ratio for 24 hours, supernatants were examined for IFN-γ secretion by ELISA. T:T2—non-specific T cells (not transduced) incubated with unloaded T2 cells; T:T2-ESO—non-specific T cells incubated with peptide-loaded T2 cells; eTCR⁺:T2—eTCR-transduced T cells incubated with unloaded T2 cells; eTCR⁺:T2-ESO—eTCR⁺ T cells incubated with peptide-loaded T2 cells.

FIGS. 22A-22D depict the persistence of eTCR-transduced cells in the blood of four patients at different time points following ACT. PBMCs obtained from each of the tested patients were stained with FITC-conjugated anti-human CD3. The CD3⁺ cell population was further stained with PE-conjugated anti-Vβ13 antibodies, and analyzed by flow cytometry. FIG. 22A—patient 1 (uveal melanoma, ACT dosage of 1×10⁹ eTCR⁺ cells). FIG. 22B—patient 2 (synovial sarcoma, ACT dosage of 1×10⁹ eTCR⁺ cells). FIG. 22C—patient 3 (Triple Negative Breast Cancer, ACT dosage of 1×10⁹ eTCR⁺ cells). FIG. 22D—patient 4 (uveal melanoma, ACT dosage of 5×10⁹ eTCR⁺ cells).

FIG. 23 depicts IFN-γ secretion levels upon co-culture of eTCR⁺ CD4⁺ cells with A375 melanoma target tumor cells expressing the NY-ESO-1 antigen (+A375) or in the absence of target cells (-A375). IFN-γ levels in the culture supernatant were determined by ELISA.

FIGS. 24A-24B depict the expression levels of the cytotoxic activation marker, CD107, in eTCR⁺CD4⁺ cells, following co-culture with A375 melanoma cells or in the absence of target cells. The transduced cells were stained with differentially labeled antibodies directed to CD107 (CD107−APC−) or the TCR β chain (Vb13.3—PE) and measured by FACS. FIG. 24A, eTCR⁺ cells incubated in the absence of target cells. FIG. 24B, eTCR⁺ cells incubated with A375 cells.

### DETAILED DESCRIPTION OF THE INVENTION

The invention relates to engineered T cell receptors (TCRs) directed to cancer testis antigen New York Esophageal Squamous Cell Carcinoma-1 (NY-ESO-1), to nucleic acid constructs encoding them, and to their use in cancer therapy. In particular, provided are isolated TCR polypeptides, as well as corresponding nucleic acid molecules and cell compositions, characterized by improved properties that are particularly adapted to cancer immunotherapy.

The invention is based, in part, on the development of improved constructs for providing NY-ESO-1-specific immunotherapy to cancer patients. As disclosed herein, a nucleic acid construct, encoding a novel recombinant (engineered) NY-ESO-1-specific TCR, was developed. Surprisingly, the construct provided for stable and prolonged expression of the TCR not only on CD8⁺ T cells, but also on the surface of CD4⁺ T cells, exhibiting exceptionally high transduction levels per transduced copy number.

Remarkably, the construct provided a significantly enhanced anti-tumor response, not only as compared to lymphocytes transduced with a non-relevant TCR, but also as compared to those transduced with a hitherto-known TCR directed to the same epitope of NY-ESO-1. Further, a clinical-grade cell composition comprising the construct was found to be highly effective and safe in the treatment of tumors in vivo, and to exhibit long-term retention in human patients.

The invention in embodiments thereof relates to an isolated TCR directed to NY-ESO-1. As disclosed herein, isolated TCRs (also referred to herein as isolated or engineered TCR complexes, molecules or polypeptides) bind specifically to an HLA-A2-presented epitope comprising the amino acid sequence SLLMWITQC (SEQ ID NO: 21), corresponding to residues 157 to 165 of human NY-ESO-1. The TCRs are characterized by a unique combination of structural elements as detailed below, providing for advantageous functional properties.

In one aspect, there is provided an isolated TCR directed to NY-ESO-1, comprising:
(a) a TCR α chain comprising: i. a variable region (VR) comprising a CDR1 having the amino acid sequence of SEQ ID NO: 1, a CDR2 having the amino acid sequence of SEQ ID NO: 2, and a CDR3 having the amino acid sequence of SEQ ID NO: 3; ii. a constant region (CR) comprising a cysteine residue at position 47 thereof and the amino acid sequence as set forth in SEQ ID NO: 22 at positions 250-254 thereof;
(b) a TCR β chain comprising: i. a VR comprising a CDR1 having the amino acid sequence of SEQ ID NO: 4, a CDR2 having the amino acid sequence of SEQ ID NO: 5, and a CDR3 having the amino acid sequence of SEQ ID NO: 6; and ii. a CR comprising a cysteine residue at position 57 thereof; and
(c) a plurality of interchain disulfide bonds between the α chain and β chain.

In another aspect, there is provided a nucleic acid construct encoding the isolated TCR. In another aspect, the invention provides a nucleic acid construct comprising at least one nucleic acid molecule selected from the group consisting of SEQ ID NOs: 19 and 20.

In another aspect, there is provided a pharmaceutical composition comprising a therapeutically effective amount of an expression vector comprising a nucleic acid construct encoding an isolated TCR directed to NY-ESO-1, comprising:
(a) a TCR α chain comprising: i. a VR comprising a CDR1 having the amino acid sequence of SEQ ID NO: 1, a CDR2 having the amino acid sequence of SEQ ID NO: 2, and a CDR3 having the amino acid sequence of SEQ ID NO: 3; ii. a CR comprising a cysteine residue at position 47 thereof and the amino acid sequence as set forth in SEQ ID NO: 22 at positions 250-254 thereof;
(b) a TCR β chain comprising: i. a VR comprising a CDR1 having the amino acid sequence of SEQ ID NO: 4, a CDR2 having the amino acid sequence of SEQ ID NO: 5, and a CDR3 having the amino acid sequence of SEQ ID NO: 6; and ii. a CR comprising a cysteine residue at position 57 thereof; and
(c) a plurality of interchain disulfide bonds between the α chain and β chain.

In another aspect, the invention relates to a cell composition for cancer immunotherapy, comprising a therapeutically effective amount of immune cells engineered to express a TCR directed to NY-ESO-1, the TCR comprising:
(a) a TCR α chain comprising: i. a VR comprising a CDR1 having the amino acid sequence of SEQ ID NO: 1, a CDR2 having the amino acid sequence of SEQ ID NO: 2, and a CDR3 having the amino acid sequence of SEQ ID NO: 3; ii. a CR comprising a cysteine residue at position 47 thereof and the amino acid sequence as set forth in SEQ ID NO: 22 at positions 250-254 thereof;
(b) a TCR β chain comprising: i. a VR comprising a CDR1 having the amino acid sequence of SEQ ID NO: 4, a CDR2 having the amino acid sequence of SEQ ID NO: 5, and a CDR3 having the amino acid sequence of SEQ ID NO: 6; and ii. a CR comprising a cysteine residue at position 57 thereof; and
(c) a plurality of interchain disulfide bonds between the α chain and β chain.

In another aspect, there is provided a process for preparing a cell composition for adoptive transfer therapy (ACT), the process comprising:
a. provided a T-cell containing cell population,
b. culturing the cells in the presence of a CD3-specific antibody and IL-2,
c. engineering the cells resulting from step b. to express the TCR as defined herein,
d. expanding the cells resulting from step c. so as to obtain a therapeutically effective amount of expanded engineered cells, and
e. harvesting the expanded engineered cells resulting from step d.

In other aspects, the invention relates to methods for treating cancer and/or for enhancing anti-tumor immunity in a subject in need thereof, the methods comprising administering to the subject a cell composition for cancer immunotherapy, comprising a therapeutically effective amount of immune cells engineered to express a TCR directed to NY-ESO-1, the TCR comprising:
(a) a TCR α chain comprising: i. a VR comprising a CDR1 having the amino acid sequence of SEQ ID NO: 1, a CDR2 having the amino acid sequence of SEQ ID NO: 2, and a CDR3 having the amino acid sequence of SEQ ID NO: 3; ii. a CR comprising a cysteine residue at position 47 thereof and the amino acid sequence as set forth in SEQ ID NO: 22 at positions 250-254 thereof;
(b) a TCR β chain comprising: i. a VR comprising a CDR1 having the amino acid sequence of SEQ ID NO: 4, a CDR2 having the amino acid sequence of SEQ ID NO: 5, and a CDR3 having the amino acid sequence of SEQ ID NO: 6; and ii. a CR comprising a cysteine residue at position 57 thereof; and
(c) a plurality of interchain disulfide bonds between the α chain and β chain.

In another aspect, the invention relates to a cell composition as defined herein for use in treating cancer in a subject in need thereof. In another aspect, the invention relates to a cell composition as defined herein for use in enhancing anti-tumor immunity in a subject in need thereof.

In other aspects, the invention relates to methods for treating cancer and/or for enhancing anti-tumor immunity in a subject in need thereof, the methods comprising administering to the subject a pharmaceutical composition comprising a therapeutically effective amount of an expression vector comprising a nucleic acid construct encoding an isolated TCR directed to NY-ESO-1, comprising:
(a) a TCR α chain comprising: i. a VR comprising a CDR1 having the amino acid sequence of SEQ ID NO: 1, a CDR2 having the amino acid sequence of SEQ ID NO: 2, and a CDR3 having the amino acid sequence of SEQ ID NO: 3; ii. a CR comprising a cysteine residue at position 47 thereof and the amino acid sequence as set forth in SEQ ID NO: 22 at positions 250-254 thereof;
(b) a TCR β chain comprising: i. a VR comprising a CDR1 having the amino acid sequence of SEQ ID NO: 4, a CDR2 having the amino acid sequence of SEQ ID NO: 5, and a CDR3 having the amino acid sequence of SEQ ID NO: 6; and ii. a CR comprising a cysteine residue at position 57 thereof; and
(c) a plurality of interchain disulfide bonds between the α chain and β chain.

In another aspect, the invention relates to a pharmaceutical composition as defined herein for use in treating cancer in a subject in need thereof. In another aspect, the invention relates to a pharmaceutical composition as defined herein for use in enhancing anti-tumor immunity in a subject in need thereof.

These and other embodiments are described in greater detail and exemplified below.

### Functional Properties

Without wishing to be bound by a specific theory or mechanism of action, TCRs and constructs of the invention are characterized by at least one and typically a plurality of properties as detailed below. In some embodiments, the properties are selected from: enhanced affinity to the target epitope, enhanced avidity, enhanced functional avidity, reduced mispairing of the α and β chains with chains of non-related (e.g. endogenous) TCRs, enhanced stability, improved transduction efficacy, increased expression levels, and/or prolonged surface expression. Accordingly, in various embodiments, compositions (including cell compositions) and methods utilizing the TCRs and constructs of the invention exhibit or provide at least one and typically a plurality of the functional properties discussed herein.

As used herein in various embodiments of the invention, the terms “enhanced”, “reduced” and the like refer to enhancement or reduction, respectively, which is recognized to be significant by the skilled artisan, in particular to statistically-significant enhancement or reduction. With respect to the functional properties of the invention, the change (e.g. enhancement, increase, improvement, reduction or prolongation) is calculated as compared to corresponding control TCR molecules, such as a naturally-occurring TCR directed to NY-ESO-1. According to particular embodiments, the change in a particular property may be conveniently calculated with respect to the value corresponding to the property exhibited by a hitherto-disclosed NY-ESO-1-specific TCR, including, but not limited to, those referred to in the Background section above. In another particular embodiment, said naturally-occurring or hitherto-disclosed TCR is directed to the NY-ESO-1 epitope of SEQ ID NO: 21. For example, without limitation, said change is conveniently calculated with respect to a TCR having VRa and VRs chains as set forth in SEQ ID NOs: 26 and 27, as disclosed and exemplified in Example 2 below.

In another embodiment, the TCR is characterized by enhanced affinity to the target epitope of SEQ ID NO: 21. Affinity is the strength of binding of one molecule to another. In particular, the term “affinity” as used herein refers to the strength of binding of an antigen molecule with an antigen-binding molecule such as an antibody or a TCR. In the context of the TCR molecules of the invention, the term “affinity” refers to the strength of a single interaction between an MHC-bound antigen (for example an NY-ESO-1 peptide epitope of SEQ ID NO: 21, presented in the context of HLA-A2), and the TCR. Affinity is measured for example where the antigen of interest is presented in a cell-free context, e.g. where the HLA-presented peptide is bound to a surface or bead. By means of non-limitative examples, the binding affinity of the TCR protein to its target may be determined by equilibrium methods (e.g. enzyme-linked immunosorbent assay (ELISA), staining with peptide/MHC multimers in the presence of competitors, Surface Plasmon Resonance (SPR), or radioimmunoassay (RIA)), or kinetics (e.g. BIACORE™ analysis).

In another embodiment, the TCR is characterized by enhanced avidity. The term “avidity” as used herein, is a measure of multiple affinities, reflecting the sum total of the strength of binding of two molecules to one another at multiple sites, e.g. taking into account the valency of the interaction. In particular, the term “avidity” as used herein refers to the overall strength of binding between an antigen with antigenic determinants (such as a peptide of interest presented by an antigen presenting cell), and an antigen-binding molecule such an a TCR or antibody (e.g. in the contest of a cell expressing the TCR or antibody). For example, with respect to the TCR molecules of the invention, the term avidity refers to the overall strength of binding between a displayed peptide such as an NY-ESO-1 epitope of SEQ ID NO: 21, presented in the context of HLA-A2, and the expressed TCR, or the overall strength of binding between a cell (e.g. tumor cell) displaying said peptide and a cell expressing said TCR. Avidity can be measured e.g. by staining TCR expressing cells (typically T cells), using graded concentrations of HLA/peptide multimers and competing or not with unlabeled HLA/peptide monomers.

In another embodiment, the TCR is characterized by enhanced functional avidity. The term “functional avidity” as used herein refers to the capability of TCR-expressing cells (in particular T cells expressing engineered TCRs as described herein, also referred to as eTCR⁺ cells) to respond in vitro to a given concentration of a ligand, and is considered to correlate with the in vivo effector capacity of TCR-expressing cells. With respect to the TCR molecules of the invention, the term “functional avidity” refers in particular to the ability of the eTCR⁺ cells to stimulate antigen-induced cytokine secretion, for example TNFα and/or IFNγ, upon co-culture with antigen-negative HLA-A2 expressing target cells (such as T2 cells) loaded with different concentration of the NY-ESO-1 peptide of SEQ ID NO: 21.

In another embodiment, the TCR is characterized by high functional avidity. By definition, TCR⁺ cells with high functional avidity respond in in vitro tests to very low antigen doses, while such cells of lower functional avidity require higher amounts of antigen before they mount an immune response similar to that of high-functional avidity TCR⁺ cells. The functional avidity can be therefore considered as a quantitative determinant of the activation threshold of a TCR⁺ cell. It is determined by exposing such cells in vitro to different amounts of cognate antigen. Accordingly, TCR⁺ cells with high functional avidity respond to low antigen doses. For example, a TCR⁺ cell will typically be considered to bind with “high” functional avidity to its antigenic target if it secretes at least about 200 pg/mL or more (e.g., 1-100, 10-90, 20-80, 10-50, 50-100, 30-70 or 40-60 ng/mL) of IFNγ upon co-culture with antigen-negative HLA-A2 expressing target cells (such as T2 cells) loaded with a low concentration of the NY-ESO-1 epitope of SEQ ID NO: 21 ranging from 10⁻⁶ to 10⁻¹² M. A TCR⁺ cell can also be considered to bind with high functional avidity to its antigenic target when exhibiting an EC50 (e.g. for IFNγ or TNFα) lower than 10⁻⁸ M and typically 10⁻¹⁰-10^{-13 M}, e.g. about 10⁻¹² M. As used herein, and unless indicated otherwise, the term “about” is meant to encompass variations of ±10%, more typically ±5%.

In some embodiments, the TCRs of the invention are capable of specific binding to an HLA-A2-presented epitope comprising the amino acid sequence SLLMWITQC (SEQ ID NO: 21) with high functional avidity. In another embodiment, the TCR is capable of specific binding to an HLA-A2-presented epitope comprising the amino acid sequence of SEQ ID NO: 21 with enhanced functional avidity as compared to a naturally occurring TCR directed to NY-ESO-1. For example, without limitation, the functional avidity may be enhanced by at least 3-fold and typically at least 4, 4.5, 5, 5.5, 6, 6.5 or 7-fold, for example 3-12-fold, 4-15-fold, 5-7-fold or 10-20-fold, as compared to the naturally occurring TCR. In various embodiments, functional avidity may be determined using well-known methods based on assays known in the art, including, but not limited to, target-induced cytokine secretion. Non-limiting examples for such assays and methods and of constructs in accordance with the invention exhibiting high functional avidity which is significantly enhanced as compared to a hitherto-known TCR directed to the same epitope, are provided in Example 2 below.

For instance, as demonstrated in Example 2, a TCR of the invention exhibited enhanced functional avidity as compared to a NY-ESO-1-specific TCR having VRα and VRβ sequences as set forth in SEQ ID NOs: 26 and 27, respectively, as determined by an antigen-induced cytokine secretion assay using at physiologically-relevant antigen levels. More specifically, enhancement of about 3-12-fold with an average of about 6-fold in TNFα secretion could be measured in the presence of NY-ESO-1 peptide concentrations of 10⁻⁶ to 10⁻⁹ M.

In another embodiment, the TCR of the invention is characterized by enhanced stability. Protein stability is an art-recognized measure of the maintenance of one or more physical properties of a protein in response to environmental conditions. For example, impairment in stability may be reflected structurally as perturbation of the primary, secondary, tertiary and/or quaternary structure of the protein (or polypeptide complex) in question. By means of non-limitative examples, protein stability may be determined by evaluating thermal stability, resistance to unfolding, renaturation yield, extended surface expression and/or specific binding of the ligand (e.g. antigen), under a variety of environmental conditions (e.g., pH, salt concentration, or the presence of detergents or denaturing agents), as known in the art.

With respect to TCR molecules of the invention, the term “stability” as used herein refers in particular to stability of the TCR complex under physiological conditions upon expression in a suitable cell or expression system. More specifically, the term relates to the ability to retain structural integrity upon expression of a TCR heterodimer of the invention on the surface of a mammalian cell, in particular a human T cell. For example, as compared to a naturally-occurring or previously disclosed TCR, a TCR of the invention may be characterized by enhanced resistance to unfolding (denaturation), enhanced resistance to misfolding of one or more of the TCR chains, reduced mispairing of the TCR heterodimer, and enhanced renaturability and/or renaturation yield. In various embodiments, enhanced stability may be reflected by an enhancement in the level, duration and/or extent of surface expression of the TCR in the absence of stabilizing molecules (such as CD8). In other embodiments, enhanced stability of the TCR complex may be reflected by enhanced antigen-binding ability in the absence of stabilizing molecules (such as CD8).

In another embodiment, the TCR is characterized by enhanced stability in the expressing cell, both in the presence and in the absence of other non-related (e.g. endogenous) TCR molecules. In another embodiment, the TCR is characterized by enhanced pairing of its α and β chains (enhancement in the proportion of specifically paired complexes out of the total produced chains). In various embodiments, enhanced pairing of the heterodimer chains is reflected by reduced mispairing of at least one of the α and β chains with non-related (e.g. endogenous) TCR chains (reduction in the proportion of mispaired complexes). In another embodiment, said TCR does not substantially yield mispaired TCR complexes upon co-expression in normal donor PBMC-derived T cells.

In another embodiment, the TCR is stably expressed on the surface of an immune cell such that it is highly active even in the absence of a CD8 molecule stabilizing the TCR-MHC complex. In another embodiment, the immune cell is a CD3⁺ cell. In another embodiment, said immune cell is a T cell. In another embodiment, said TCR is capable of being stably expressed on the surface of CD8⁻ immune cells, such as on helper T cells. In another embodiment the encoded TCR is capable of being stably expressed on both CD4⁺ and CD8⁺ T cells (with adequate stability and functionality so as to allow an antigen-specific response under physiologically relevant conditions).

In another embodiment, the TCR is able to exert tumor-specific reactivity (e.g. cytotoxicity) in a CD8-independent manner (that does not require co-expression or prior expression of CD8 by the TCR-expressing cell to exert said function or activity). Thus, a T cell expressing a TCR of the invention is capable of eliciting or enhancing a TCR-mediated functional activity (such as cytotoxicity or cytokine production), specifically against an NY-ESO-1-expressing tumor, as compared to their response to non-related cells (not expressing the antigen, which are typically and advantageously unaffected). Remarkably, as disclosed herein, the TCRs of the invention are capable not only of being expressed on the surface of CD8⁻ immune cells (such as CD4⁺ cells), but also of retaining the antigen-specific activity of the TCR molecules when expressed in these cells. Thus, as exemplified herein, TCRs of the invention, when expressed on the surface of CD4⁺ T cells, were functionally reactive against NY-ESO-1—expressing tumor cells, and showed the ability to secrete high levels of IFN-γ in response to target melanoma cells (A375) expressing the antigen. Further, as demonstrated herein, CD4⁺ TCR⁺ cells prepared in accordance with the invention were demonstrated to adopt a cytotoxic phenotype, characterized by enhanced expression of the cytotoxic effecter marker CD107 (CD107⁺), which was further enhanced upon recognition with the cognate target A375 tumor cells.

In some embodiments, TCRs of the invention may be stably expressed on the surface of both CD4⁺ and CD8⁺ T cells, such that both populations are capable of in vitro and/or in vivo clonal (antigen-specific) expansion. Thus, a T cell population derived from T cells engineered (e.g. transduced) to express a TCR of the invention, in particular cell compositions prepared from healthy human donor PBMCs according to the processes as disclosed herein, may advantageously exhibit an enhancement in the relative proportion of eTCR⁺ cells over time in the presence of their cognate antigen (and in the absence of subsequent engineering steps). For example, without limitation, such enhancement may be observed within 20-60, 30-50 or 35-45 days post-transduction (or otherwise engineering), or 15-40, 17-35 or 19-30 post adoptive transfer. By means of a non-limitative example, in vivo stability, retention and clonal expansion may conveniently be evaluated in suitable animal models accepted in the art, such as in NOD SCID gamma (NSG) mice bearing NY-ESO-1-expressing tumor xenografts, e.g. as described in Example 6 below. According to exemplary embodiments, compositions in accordance with the invention may be characterized by an average expression of said TCR of at least 97%, typically at least 98% and more typically at least 99% on CD8⁺ cells 40 days after transduction (or otherwise engineering, e.g. in the NSG xenograft model adoptively transferred with healthy human donor PBMC-derived cell compositions as discussed herein). In addition, compositions in accordance with the invention may be characterized by an average expression of said TCR of at least 30%, typically at least 35% and more typically at least 40% on CD4⁺ cells 40 days after transduction (or otherwise engineering, e.g. in the NSG xenograft model adoptively transferred with healthy human donor PBMC-derived cell compositions as discussed herein).

In another embodiment the invention provides for an enhanced level and/or duration of expression of the encoded TCR, as compared to a construct encoding a naturally-occurring TCR directed to an HLA-A2-presented epitope as set forth in SEQ ID NO: 21. In a particular embodiment, the invention provides for enhancing the proportion of cells characterized by surface expression of said TCR as compared to a corresponding construct encoding a naturally-occurring TCR directed to an HLA-A2-presented epitope as set forth in SEQ ID NO: 21. In another embodiment, the invention provides for the production of adoptive cell therapy compositions characterized by surface expression of said TCR which is greater than 50% of the engineered cell population, and typically by an average expression greater than 53%, 55%, 60%, 70%, 80%, 90% or more, as disclosed herein.

In another embodiment, the invention provides for high TCR expression levels per construct copy number. The term “copy number” is used herein in connection with nucleic acid constructs (optionally as “CPN”, “construct copy number”, “vector copy number” or “VCN”) to indicate the number of copies of the construct, e.g. a plasmid or vector (such as a viral vector), per cell. The copy number may be determined for individual cells, and also for a population of cells. In the latter case, an average CPN is obtained. Exemplary methods for measuring CPN include PCR procedures (e.g. quantitative polymerase chain reaction (qPCR) and digital droplet polymerase chain reaction (ddPCR)) and flow cytometry. As there may be an increased risk of oncogenesis if the vector copy number (VCN) per cell is high (in particular when using genome-integrating vectors), administration (FDA) recommends that the VCN shall be <5 copies per genome.

According to exemplary embodiments, the invention enables the production of immune cell compositions in which at least 60%, 70%, 80% or 90% of the immune cells (e.g. derived from healthy donor PBMC) are characterized by surface expression of the TCR and by an average copy number of five or less molecules per cell of the construct encoding said TCR. In contradistinction, hitherto reported approaches provide for an average expression level of 30-50% in a cell composition comprising an equivalent average copy number of the corresponding construct. In another embodiment, the functional property is as detailed and exemplified herein. Each possibility represents a separate embodiment of the invention.

### Structural Properties

TCRs are disulfide-linked membrane-bound heterodimeric proteins expressed on the surface of T cells, which are members of the immunoglobulin superfamily. TCRs engage, via their variable regions, antigenic peptide in complex with the MHC/HLA, to induce downstream T cell signaling (further referred to herein as TCR-mediated signaling). TCRs typically comprise the highly variable alpha and beta chains, which complex with invariant CD3 chain molecules; a minority of TCRs comprise variable gamma and delta chains. Each of the alpha (α) chain and the beta (β) chain comprises two extracellular domains: a variable region (VR) and a constant region (CR). Each variable region (e.g. in the α chain and the β chain) contains three hypervariable regions, also referred to as “complementarity determining regions” (CDRs) separated by framework regions (FRs). CDR3 is the main CDR responsible for antigen binding. The α and β chains also contain joining (J) regions. The β chain also usually contains a diversity (D) region between the V and J regions; however, this D region may be considered part of the J region.

In naturally-occurring TCRs, the constant region is encoded by the TRAC gene for TCR α chains and either the TRBC1 or TRBC2 genes for TCR β chains, whereas the TCR variable regions are encoded by TRAV or TRAJ gene segments for TCR α chains and by TRBV, TRBD or TRBJ gene segments for a TCR β chains. During thymocyte development, TCR gene segments are assembled by processes including random somatic gene rearrangement (V(D)J recombination) combined with a selection process for proper expression and MHC recognition, to generate the diverse T cell repertoire characteristic of the individual.

Specific MHC-presented antigen recognition and binding by the clonotype-specific α/β heterodimer leads to activation of transcriptional events in the expressing T cells, and subsequent proliferation and effector functions (such as cytotoxic activity in CD8⁺ T cells and cytokine secretion in CD4⁺ T cells). This activation involves other subunits (e.g., CD3) of the receptor complex that couple the extracellular liganding event to downstream signaling pathways (TCR-mediated signaling, e.g. protein phosphorylation, the release of inositol phosphates and the elevation of intracellular calcium levels.

As disclosed herein, the invention relates to improved TCR molecules, characterized by structural properties as detailed hereinabove. In particular, provided are TCR complexes which are heterodimers of structurally-modified (non-naturally occurring) alpha and beta chains (also referred to as “engineered” or “recombinant” α/β TCRs). More specifically, TCRs in accordance with the invention comprise complementarity determining region (CDR) sequences as set forth in Table 1 below, in which CDR₁α, CDR₂α and CDR₃α correspond to the CDR1, CDR2 and CDR3 sequences of the alpha chain, and CDR₁β, CDR₂β and CDR₃β correspond to the CDR1, CDR2 and CDR3 sequences of the beta chain, respectively. In particular, TCRs in accordance with the invention employ the use of an improved CDR₃α, as disclosed herein. In addition, advantageous nucleic acid sequences encoding the CDRs that are particularly suitable for use in nucleic acid constructs for cancer immunotherapy, are further listed in Table 1.

TABLE 1
CDR sequences

| | Amino acid | | | |
|---|---|---|---|---|
| | sequence/ | | | |
| CDR | SEQ ID NO. | | Nucleic acid sequence/SEQ ID NO. | |
| CDR₁α | DSAIYN | 1 | GACTCTGCCATCTACAAC | 7 |
| CDR₂α | IQSSQRE | 2 | ATCCAGAGCTCGCAGAGGGAG | 8 |
| CDR₃α | AVRPLYGGSYIPT | 3 | GCCGTGCGGCCTCTGTACGGAGGCTCTTATATCCCAACC | 9 |
| CDR₁β | MNHEY | 4 | ATGAATCACGAGTAC | 10 |
| CDR₂β | SVGAGI | 5 | TCCGTGGGAGCAGGAATC | 11 |
| CDR₃β | ASSYVGNTGELF | 6 | GCCTCCTCTTATGTGGGCAACACCGGCGAGCTGTTC | 12 |

As further disclosed herein, TCRs in accordance with the invention comprise a plurality of interchain disulfide bonds. As used herein the term “disulfide bond” (also known as a disulfide bridge) includes the covalent bond formed between two sulfur atoms. The amino acid cysteine comprises a thiol group that can form a disulfide bond or bridge with a second thiol group. Naturally-occurring TCR complexes typically contain a single disulfide bond between their TCR chains, formed by the presence of a cysteine residue at specific locations of the constant region (CR) of each of the chains. In contradistinction, TCRs of the invention contain a plurality of disulfide bonds, including, in particular, an engineered (artificial) disulfide bridge as disclosed herein. The term “interchain disulfide bond” as used herein thus refers to any disulfide bridge, including both natural and engineered or artificial disulfide bridges, formed between cysteine residues located in the CRs of the TCR α chain and TCR β chain, respectively.

In particular, TCRs of the invention further comprise a second disulfide bond between the α and β chains, facilitated by substitutions of particular amino acids at both chains into cysteine residues. In some embodiments, the second disulfide bond has no equivalent in native α/β TCRs, yet provides for the variable domain sequences of the α and β chains to be mutually orientated substantially as in native α/β TCRs. Thus, the engineered TCRs of the invention have modified stability but retain and even improve their functionality, for example, their specificity towards their cognate antigen and their ability to induce TCR-mediated signaling and activate T cells.

In particular, disclosed herein are TCRs comprising cysteine residues at position 182 of the α chain precursor and position 189 of the β chain precursor, forming a disulfide bond (in the mature TCR complex). It is to be understood, that the positions mentioned above are presented with respect to the α and β chain precursors, including their respective signal peptides. Thus, the above-mentioned cysteine residues correspond to positions 47 and 57 of the CR of the α and β chains, respectively.

In another embodiment, the TCR further comprises a disulfide bond between the cysteine residues at positions 229 of the α chain precursor and position 263 of the β chain precursor. In some embodiments, the plurality of disulfide bonds comprises, or consists of, a first and a second disulfide bonds as disclosed herein. In another embodiment, the TCR comprises a first disulfide bond between the cysteine residues at positions 94 of the α chain CR and 131 of the β chain CR, and a second disulfide bond between the cysteine residues at positions 47 of the α chain CR and 57 of the β chain CR.

For example, the TCR α chain precursor of SEQ ID NO: 17 as set forth below comprises cysteine residues at positions 182 and 229 thereof (considering the signal peptide at positions 1-20), which correspond to positions 47 and 94 of its CR sequence as set forth in SEQ ID NO: 14 below. Similarly, in another example, the β chain precursor of SEQ ID NO: 18 as set forth below comprises cysteine residues at positions 189 and 263, which correspond to positions 57 and 131 of its CR sequence as set forth in SEQ ID NO: 16 below. According to embodiments of the invention, these cysteine residues are involved in forming disulfide bonds, in particular interchain disulfide bonds as disclosed herein.

As also disclosed herein, TCRs in accordance with the invention are characterized by an enhanced incidence of hydrophobic amino acids. The term “hydrophobic amino acid,” as used herein, refers to an amino acid having a side chain that is uncharged at physiological pH, is not polar, and is generally repelled by aqueous solution. Exemplary hydrophobic amino acids include isoleucine (Ile or I), phenylalanine (Phe or F), valine (Val or V), leucine (Leu or L), tryptophan (Trp or W), methionine (Met or M), alanine (Ala or A), and glycine (Gly or G). Particularly preferred hydrophobic amino acids in the present disclosure are leucine, isoleucine, and valine. In another embodiment, the hydrophobic amino acids are selected from the group consisting of alanine, glycine, leucine, isoleucine, and valine.

In particular, provided are TCRs in which the α chain comprises a LVIVL (SEQ ID NO: 22) sequence. Typically and advantageously, a sequence as set forth in SEQ ID NO: 22 is located at the transmembrane region of the α chain. More specifically, TCRs of the invention comprise a LVIVL sequence located at positions 250-254 of the α chain precursor (including the signal peptide). Thus, TCRs of the invention may comprise a LVIVL sequence at positions 115-119 of the α chain CR (CRα). In another embodiment, the transmembrane region of the α chain comprises at least 14 hydrophobic amino acids. As used herein, the term “transmembrane” with respect to a TCR chain refers to the portion or portions of a TCR chain that are embedded in the plasma membrane of a cell. Transmembrane regions or domains of TCR chains are typically about 20-25 amino acids (aa) in length (e.g. 21), and comprise a transmembrane alpha helix and several conserved polar residues. According to embodiments of the invention, a TCR chain in accordance with the invention comprises a TCR-derived transmembrane domain (e.g. derived from a naturally occurring TCR chain) modified to include the hydrophobic sequences and/or residues as disclosed herein. In another embodiment, the transmembrane region of the α chain has the amino acid sequence LLVIVLRILLLKVAGFNLLMT (SEQ ID NO: 23).

In addition, TCR chain precursors of the invention typically contain modified signal peptides. As used herein, the term “signal peptide” has its general meaning in the art and refers to a pre-peptide which is present as an N-terminal peptide on a precursor form of a protein and directs the polypeptide into the cell's secretory pathway. Signal peptides are typically cleaved from a protein during translation or transport, and are therefore not present in the mature protein. In one embodiment, a signal peptide as used herein has a modified sequence (not characteristic of naturally-occurring TCR chains), yet provides for adequate presentation of the TCR chain on the plasma membrane. In particular, an α chain precursor in accordance with the invention may comprise a phenylalanine residue at position 2 of the signal peptide.

Accordingly, amino acid sequences of advantageous TCRs in accordance with the invention are provided in SEQ ID NOs: 13-18 below, as follows:

Variable region of the α chain
(VRα, excluding the signal peptide):
(SEQ ID NO: 13)
KQEVTQIPAALSVPEGENLVLNCSFTDSAIYNLQWFRQDPGKGLTSLLLI
QSSQREQTSGRLNASLDKSSGRSTLYIAASQPGDSATYLCAVRPLYGGSY
IPTFGRGTSLIVHPY.
Constant region of the α chain (CRα):
(SEQ ID NO: 14)
IQNPDPAVYQLRDSKSSDKSVCLFTDFDSQTNVSQSKDSDVYITDKCVLD
MRSMDFKSNSAVAWSNKSDFACANAFNNSIIPEDTFFPSPESSCDVKLVE
KSFETDINLNFQNLLVIVLRILLLKVAGFNLLMTLRLWSS.
Variable region of the β chain
(VRβ, excluding the signal peptide):
(SEQ ID NO: 15)
GVTQTPKFQVLKTGQSMTLQCAQDMNHEYMSWYRQDPGMGLRLIHYSVGA
GITDQGEVPNGYNVSRSTTEDFPLRLLSAAPSQTSVYFCASSYVGNTGEL
FFGEGSRLTVL.
Constant region of the β chain (CRβ):
(SEQ ID NO: 16)
EDLNKVFPPEVAVFEPSEAEISHTQKATLVCLATGFFPDHVELSWWVNGK
EVHSGVCTDPQPLKEQPALNDSRYCLSSRLRVSATFWQNPRNHFRCQVQF
YGLSENDEWTQDRAKPVTQIVSAEAWGRADCGFTSVSYQQGVLSATILYE
ILLGKATLYAVLVSALVLMAMVKRKDF.

The amino acid sequences of the full-length α and β chain precursors (including the signal peptides, which are underlined) in accordance with particularly advantageous embodiments of the invention are as set forth in SEQ ID NOs: 17-18, respectively, as follows:

(α chain precursor,)
SEQ ID NO: 17
MFETLLGLLILWLQLQWVSSKQEVTQIPAALSVPEGENLVLNCSFTDSAI
YNLQWFRQDPGKGLTSLLLIQSSQREQTSGRLNASLDKSSGRSTLYIAAS
QPGDSATYLCAVRPLYGGSYIPTFGRGTSLIVHPYIQNPDPAVYQLRDSK
SSDKSVCLFTDFDSQTNVSQSKDSDVYITDKCVLDMRSMDFKSNSAVAWS
NKSDFACANAFNNSIIPEDTFFPSPESSCDVKLVEKSFETDINLNFQNLL
VIVLRILLLKVAGFNLLMTLRLWSS.
(β chain precursor,)
SEQ ID NO: 18
MSIGLLCCAALSLLWAGPVNAGVTQTPKFQVLKTGQSMTLQCAQDMNHEY
MSWYRQDPGMGLRLIHYSVGAGITDQGEVPNGYNVSRSTTEDFPLRLLSA
APSQTSVYFCASSYVGNTGELFFGEGSRLTVLEDLNKVFPPEVAVFEPSE
AEISHTQKATLVCLATGFFPDHVELSWWVNGKEVHSGVCTDPQPLKEQPA
LNDSRYCLSSRLRVSATFWQNPRNHFRCQVQFYGLSENDEWTQDRAKPVT
QIVSAEAWGRADCGFTSVSYQQGVLSATILYEILLGKATLYAVLVSALVL
MAMVKRKDF.

Thus, the amino acid sequences of the mature, processed TCR chains (lacking their respective signal peptides) correspond to positions 21-275 of SEQ ID NO: 17 and positions 22-309 of SEQ ID NO: 18 for the α and β chains, respectively. As used herein, the term “TCR chain precursor” refers to the recombinant molecule formed upon translation in a suitable host cell or expression system, prior to subsequent processing such as cleavage of the signal peptide, glycosylation, complexing with the corresponding chain (e.g. α-β chain alignment) and formation of disulfide bonds. It is to be understood that this term as used herein is not meant to include or necessitate pre-translational processing such as somatic recombination.

### Nucleic Acid Sequences

Further, TCRs in accordance with the invention are advantageously encoded by improved nucleic acid sequences. Without wishing to be bound by a specific theory or mechanism of action, nucleic acid sequences employed by embodiments of the invention provide for improved expression in mammalian cells and expression systems, in particular in human cells such as lymphocytes.

The nucleic acid sequences encoding for the full-length α and β chain precursors (including the signal peptides, which are underlined) in accordance with particularly advantageous embodiments of the invention are as set forth in SEQ ID NOs: 19-20, respectively, as follows:

Nucleic acid molecule encoding
TCRα precursor -
(SEQ ID NO: 19)
ATGTTCGAAACTCTGCTGGGGCTGCTGATTCTGTGGCTGCAGCTGCAGTG
GGTGTCATCCAAACAGGAGGTCACTCAGATTCCCGCTGCCCTGAGCGTGC
CTGAGGGCGAGAACCTGGTGCTGAATTGCTCCTTCACCGACTCTGCCATC
TACAACCTGCAGTGGTTTAGGCAGGATCCAGGCAAGGGCCTGACCAGCCT
GCTGCTGATCCAGAGCTCGCAGAGGGAGCAGACATCCGGCCGCCTGAATG
CCTCTCTGGACAAGTCTAGCGGCCGGAGCACCCTGTACATCGCAGCAAGC
CAGCCAGGCGATTCCGCCACATACCTGTGCGCCGTGCGGCCTCTGTACGG
AGGCTCTTATATCCCAACCTTCGGCAGAGGCACAAGCCTGATCGTGCACC
CTTACATCCAGAACCCAGACCCCGCCGTGTATCAGCTGCGGGACAGCAAG
TCCTCTGATAAGTCCGTGTGCCTGTTCACCGACTTTGATTCCCAGACAAA
CGTGAGCCAGAGCAAGGACTCTGACGTGTACATCACCGACAAGTGCGTGC
TGGATATGAGAAGCATGGACTTTAAGTCCAACTCTGCCGTGGCCTGGAGC
AATAAGTCCGATTTCGCCTGCGCCAACGCGTTTAACAATAGCATCATCCC
CGAGGATACATTCTTTCCTTCCCCAGAGAGCTCCTGTGACGTGAAGCTGG
TGGAGAAGAGCTTCGAGACAGATACAAACCTGAATTTTCAGAACCTGCTG
GTCATCGTGCTGCGGATCCTGCTGCTGAAGGTGGCCGGCTTCAATCTGCT
GATGACCCTGAGACTGTGGTCTAGC.

Thus, the VRα and CRα are encoded by positions 1-405 and 406-825 of SEQ ID NO: 19, respectively.

Nucleic acid molecule encoding
TCRβ precursor -
(SEQ ID NO: 20)
ATGTCTATCGGCCTGCTGTGCTGTGCCGCCCTGAGCCTGCTGTGGGCAGG
ACCAGTGAACGCAGGAGTGACCCAGACACCCAAGTTCCAGGTGCTGAAGA
CCGGCCAGTCTATGACACTGCAGTGCGCCCAGGACATGAATCACGAGTAC
ATGAGCTGGTATCGGCAGGATCCTGGCATGGGCCTGAGACTGATCCACTA
CTCCGTGGGAGCAGGAATCACCGACCAGGGAGAGGTGCCAAACGGCTATA
ACGTGAGCAGGAGCACCACAGAGGATTTCCCACTGAGGCTGCTGTCTGCC
GCACCTTCTCAGACAAGCGTGTACTTTTGCGCCTCCTCTTATGTGGGCAA
CACCGGCGAGCTGTTCTTTGGCGAGGGCTCCAGGCTGACAGTGCTGGAGG
ACCTGAATAAGGTGTTCCCCCCTGAGGTGGCCGTGTTTGAGCCCTCTGAG
GCCGAGATCAGCCACACCCAGAAGGCCACCCTGGTGTGCCTGGCAACCGG
CTTCTTTCCTGATCACGTGGAGCTGTCCTGGTGGGTGAACGGCAAGGAGG
TGCACTCTGGCGTGTGCACAGACCCACAGCCCCTGAAGGAGCAGCCAGCC
CTGAATGACTCgAGATACTGCCTGAGCAGCCGCCTGAGGGTCTCGGCCAC
CTTCTGGCAGAACCCCCGCAACCACTTCCGCTGTCAAGTCCAGTTCTACG
GGCTCTCGGAGAATGACGAGTGGACCCAGGATAGGGCCAAACCCGTCACC
CAGATCGTCAGCGCCGAGGCCTGGGGTAGAGCAGACTGTGGCTTTACCTC
GGTGTCCTACCAGCAAGGGGTCCTGTCTGCCACCATCCTCTATGAGATCC
TGCTAGGGAAGGCCACCCTGTATGCTGTGCTGGTCAGCGCCCTTGTGTTG
ATGGCCATGGTCAAGAGAAAGGATTTCTGA.

Thus, the VRβ and CRβ are encoded by positions 1-396 and 397-930 of SEQ ID NO: 20, respectively.

In other embodiments, the invention relates to nucleic acid sequences having a high degree of homology (e.g. at least 95%, 96%, 97%, 98% or 99% sequence identity and/or homology to SEQ ID NOs: 19 or 20), wherein the resulting TCR is capable of specific binding to an HLA-A2-presented epitope of SEQ ID NO: 21 with high functional avidity. Each possibility represents a separate embodiment of the invention. Such nucleic acid molecules typically_comprise the nucleic acid sequences as set forth in SEQ ID NOs: 7-9, encoding the CDR1, CDR2 and CDR3 of the α chain, respectively, or the nucleic acid sequences as set forth in SEQ ID NOs: 10-12, encoding the CDR1, CDR2 and CDR3 of the β chain, respectively.

In another embodiment, the nucleic acid molecule of methods and compositions of the present invention is a DNA molecule. In another embodiment, the nucleic acid molecule is an RNA molecule. In another embodiment, the molecule is any other type of nucleic acid molecule known in the art. Each possibility represents a separate embodiment of the present invention.

### TCRs

The invention in aspects and embodiments thereof relate to isolated TCRs directed to NY-ESO-1. The term “isolated” as used herein, means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

The term “directed” as used herein in connection with antigen-binding proteins such as TCRs and antibodies refers to the ability to specifically bind said antigen under physiological conditions. As used herein, “specifically binds” or “specific for” refers to an association or union of a binding protein (e.g., TCR) to a target molecule (e.g. MHC-presented peptide epitope) with an affinity or Kₐ(i.e., an equilibrium association constant of a particular binding interaction with units of 1/M) equal to or greater than 10⁵ M⁻¹, while not significantly associating or uniting with any other molecules or components in a sample. Binding proteins or binding domains may be classified as “high-affinity” or as “low-affinity” binding proteins or domains. “High-affinity” binding proteins or binding domains refer to those molecules having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low-affinity” binding proteins or binding domains refer to those molecules having a Kₐ of up to up to 10⁶ M⁻¹, or up to 10⁵ M⁻¹.

The term “NY-ESO-1” or “New York esophageal squamous cell carcinoma 1” refers to the well-known cancer-testis antigen (CTA) also known as cancer/testis antigen 1B (CTAGIB). The human CTAG1B gene maps to the Xq28 region of the X chromosome, and is silenced in normal somatic cells except for male testis. However, NY-ESO-1 is aberrantly expressed in many types of cancer cells as a consequence of an epigenetic event that involves tightly controlled recruitment and sequential interaction of histone deacetylases, histone methyltransferase, DNA methyltransferases, and transcription factors. A human NY-ESO-1 sequence is set forth in accession no. NP_001318.1.

More specifically, TCRs in accordance with the invention bind specifically to an HLA-A2-presented epitope comprising the amino acid sequence of SEQ ID NO: 21, corresponding to residues 157 to 165 of human NY-ESO-1. According to particular embodiments, TCRs of the invention are directed to a peptide epitope of SEQ ID NO: 21 in the context of HLA-A*0201 and/or HLA-A*0206. As used herein, the term “HLA-presented” peptide, epitope or antigen refers to a peptide capable of specifically binding an antigen-binding groove of an MHC or a particular allele thereof (for example an HLA-A2-presented epitope is specific to HLA-A2 alleles). Such an antigen is commonly referred to in the art as being “restricted” by such an MHC. The antigen generally has a characteristic dimension and/or chemical composition—for example, a characteristic amino acid length and set of anchor residues, respectively, in the case of a peptide antigen—enabling it to specifically bind the antigen-binding groove of a particular MHC haplotype so as to form an MHC/antigen complex therewith having an antigen presenting portion capable of specifically binding a variable region of a cognate TCR. For HLA-A2, for example, the anchoring positions are P2 and P9.

In one aspect, the invention relates to an isolated TCR directed to NY-ESO-1, comprising:
(a) a TCR α chain comprising:
   i. a CDR1 having the amino acid sequence of SEQ ID NO: 1, a CDR2 having the amino acid sequence of SEQ ID NO: 2, and a CDR3 having the amino acid sequence of SEQ ID NO: 3;
   ii. a CR comprising a cysteine residue at position 47 thereof and a LVIVL sequence at positions 250-254 thereof;
   iii. optionally a signal peptide comprising a phenylalanine residue at position 2;
(b) a TCR β chain comprising:
   i. a CDR1 having the amino acid sequence of SEQ ID NO: 4, a CDR2 having the amino acid sequence of SEQ ID NO: 5, and a CDR3 having the amino acid sequence of SEQ ID NO: 6; and
   ii. a CR comprising a cysteine residue at position 57 thereof;
   iii. optionally a signal peptide; and
(c) a plurality of interchain disulfide bonds between said α chain and β chain.

In another aspect, the invention provides isolated TCR directed to NY-ESO-1, comprising:
(a) a TCR α chain comprising:
   i. a VR comprising a CDR1 having the amino acid sequence of SEQ ID NO: 1, a CDR2 having the amino acid sequence of SEQ ID NO: 2, and a CDR3 having the amino acid sequence of SEQ ID NO: 3;
   ii. a CR comprising a cysteine residue at position 47 thereof and the amino acid sequence as set forth in SEQ ID NO: 22 at positions 250-254 thereof;
(b) a TCR β chain comprising:
   i. a VR comprising a CDR1 having the amino acid sequence of SEQ ID NO: 4, a CDR2 having the amino acid sequence of SEQ ID NO: 5, and a CDR3 having the amino acid sequence of SEQ ID NO: 6; and
   ii. a CR comprising a cysteine residue at position 57 thereof; and
(c) a plurality of interchain disulfide bonds between the α chain and β chain.

In another embodiment, the CDRs are as set forth in SEQ ID NOs: 1-6. In another embodiment the TCR comprises a TCR α chain having an amino acid sequence as set forth in SEQ ID NO: 17, optionally excluding the signal peptide at positions 1-20. In another embodiment the TCR comprises a TCR β chain having an amino acid sequence as set forth in SEQ ID NO: 18, optionally excluding the signal peptide at positions 1-21. In another embodiment, said TCR comprises the α and β chains as set forth in SEQ ID NOs: 17 and 18, optionally excluding their respective signal peptides.

In yet other embodiments, the invention encompasses TCRs comprising certain modifications (e.g. substitutions) to the amino acid sequences as set forth in SEQ ID NOs: 17 and 18 which retain a high degree of homology (e.g. greater than 95%, 96% 97%, 98% or 99%), as long as the structural elements as set forth at clauses (a) to (c) above are maintained. In a particular embodiment, the substitutions are conservative substitutions.

The terms “homology” and “sequence identity” as used herein refer to the degree of relatedness between two or more amino acid sequences, or two or more nucleic acid sequences, as determined by comparing the sequences. The comparison of sequences and determination of sequence identity or homology may be accomplished using a mathematical algorithm; those skilled in the art will be aware of computer programs available to align two sequences and determine the percent identity between them. The term “homology” refers in particular to the percentage of amino acid residues or nucleotides in a sequence that are identical with the residues of the reference polypeptide or polynucleotide with which it is compared, after aligning the sequences and in some embodiments after introducing gaps, if necessary, to achieve the maximum percentage homology, and not considering any conservative substitutions. As used here, the term “% identity,” which may be used interchangeably with the term “sequence identity”, in the context of two or more nucleic acid or polypeptide sequences, refer to two or more sequences that have a specified percentage of nucleotides or amino acid residues that are the same, when compared and aligned for maximum correspondence, as measured using a suitable sequence comparison algorithm (e.g., BLASTP and BLASTN or other algorithms available to persons of skill) or by visual inspection. Further, with respect to sequence identity as used herein, the overall length of the molecules compared is also taken into consideration, such that the degree of identity is calculated over the entire length of the sequences rather than locally.

As disclosed herein, a TCR retaining the unique structural elements of the present disclosure, in particular those set forth at clauses (a) to (c) while retaining a high degree of homology and/or identity, manifest advantageous functional properties as disclosed in the present specification, such as their epitope specificity and functional avidity. The term “high” with respect to sequence homology or identity refers to at least 90% homology or sequence identity, respectively, e.g. at least 95%, 96% 97%, 98% or 99% homology or identity, wherein each possibility represents a separate embodiment of the invention. In another particular embodiment, each of the α and β chains exhibits at least 95% sequence identity to SEQ ID NOs: 17 and 18, respectively, and said TCR is capable of specific binding to an HLA-A2-presented epitope comprising the amino acid sequence SLLMWITQC (SEQ ID NO: 21) with high functional avidity. In various other particular embodiments, each of the α and β chains exhibits at least 95%, 96%, 97%, 98% or 99% sequence identity and/or homology to SEQ ID NOs: 17 and 18, respectively, such that the resulting TCR is capable of specific binding to an HLA-A2-presented epitope of SEQ ID NO: 21 with high functional avidity. Each possibility represents a separate embodiment of the invention.

### Constructs and Vectors

Polypeptides, peptides and nucleic acid molecules may conveniently be produced by recombinant technology. Recombinant methods for designing, expressing and purifying proteins, peptides and nucleic acid molecules are known in the art (see, e.g. Sambrook et al., 2001, Molecular Cloning: A Laboratory Manual, Cold Springs Harbor Laboratory, New York). Nucleic acid molecules may include DNA, RNA, or derivatives of either DNA or RNA. An isolated nucleic acid sequence encoding a polypeptide or peptide can be obtained from its natural source, either as an entire (i.e., complete) gene or a portion thereof. A nucleic acid molecule can also be produced using recombinant DNA technology (e.g., polymerase chain reaction (PCR) amplification, cloning) or chemical synthesis. Nucleic acid sequences include natural nucleic acid sequences and homologs thereof, including, but not limited to, modified nucleic acid sequences in which nucleotides have been inserted, deleted, substituted, and/or inverted in such a manner that such modifications do not substantially interfere with the nucleic acid molecule's ability to encode a functional product. A polynucleotide or oligonucleotide sequence can be deduced from the genetic code of a protein, however, the degeneracy of the code must be taken into account, as well as the allowance of exceptions to classical base pairing in the third position of the codon, as given by the so-called “Wobble rules”. Polynucleotides that include more or less nucleotides can result in the same or equivalent proteins. Using recombinant production methods, selected host cells, e.g. of a microorganism such as E. coli or yeast, are transformed with a hybrid viral or plasmid DNA vector including a specific DNA sequence coding for the polypeptide and the polypeptide is synthesized in the host upon transcription and translation of the DNA sequence.

Such recombinant methods may also be used in the preparation of nucleic acid constructs, including in particular expression constructs or vectors used for delivering and expressing the TCRs of the invention in a mammalian host such as a human T cell, as detailed herein. The constructs comprise nucleic acid molecules of the invention, and may also comprise regulatory sequences or selectable markers, as known in the art. The nucleic acid construct (also referred to in some embodiments as a vector) may include additional sequences that render this vector suitable for replication and integration in prokaryotes, eukaryotes, or optionally both (e.g., shuttle vectors). In addition, a typical cloning vector may also contain transcription and translation initiation sequences, transcription and translation terminators, and a polyadenylation signal.

In another aspect, there is provided a nucleic acid construct encoding at least one chain of the isolated TCR. In another embodiment, the nucleic acid construct comprises at least one nucleic acid molecule encoding the TCR α and β chains as disclosed herein. In another embodiment, the at least one nucleic acid molecule is operatively linked to one or more transcription control elements.

The phrase “operably linked” refers to a nucleic acid sequence linked a to a transcription control sequence in a manner such that the molecule is able to be expressed when transfected (i.e., transformed, transduced, infected, or transfected) into a host cell. Transcription control sequences are sequences, which control the initiation, elongation, and termination of transcription. Particularly important transcription control sequences are those which control transcription initiation, such as promoter, enhancer, operator and repressor sequences.

In another embodiment, the construct comprises the nucleic acid sequences as set forth in SEQ ID NOs: 7-9, encoding the CDR1, CDR2 and CDR3 of the α chain, respectively, and the nucleic acid sequences as set forth in SEQ ID NOs: 10-12, encoding the CDR1, CDR2 and CDR3 of the β chain, respectively. In another embodiment the construct comprises a first nucleic acid molecule encoding the α chain and having the nucleic acid sequence as set forth in SEQ ID NO: 19, and a second nucleic acid molecule encoding the β chain and having the nucleic acid sequence as set forth in SEQ ID NO: 20. In another embodiment, there is provided a nucleic acid construct comprising at least one nucleic acid molecule selected from the group consisting of SEQ ID NOs: 19 and 20.

In another embodiment, said construct comprises a first nucleic acid molecule encoding a TCR α chain and a second nucleic acid molecule encoding a TCR β chain, said first and second nucleic acid molecules having the nucleic acid sequences as set forth in SEQ ID NOs: 19-20, respectively, and wherein said first and second nucleic acid molecules are operatively linked to one or more transcription control elements. In another embodiment, the first nucleic acid molecule is 5′ to the second nucleic acid molecule. In another embodiment, the first nucleic acid molecule is 3′ to the second nucleic acid molecule. In another embodiment, the one or more transcription control elements (e.g. promoters) are capable of inducing or enhancing the expression of said TCR chains in a human leukocyte cell. In another embodiment, the one or more transcription control elements (e.g. promoters) are capable of inducing or enhancing the expression of said TCR chains in a human lymphocyte, e.g. T cell.

For example, in an expression vector capable of expressing a nucleic acid molecule in human T cells, expression control sequences operatively linked to the gene product to be expressed include promoters and other elements that are active in human T cells. The promoter can be of genomic origin or synthetically generated. A variety of promoters for use in T cells are well-known in the art. The promoter can be constitutive or inducible, where induction is associated with the specific cell type or a specific level of activation or maturation, for example. Alternatively, a number of well-known viral promoters are also suitable. Promoters of interest include but are not limited to the β-actin promoter, SV40 early and late promoters, immunoglobulin promoter, human cytomegalovirus promoter, retrovirus promoter, and the Friend spleen focus-forming virus promoter. The promoters may or may not be associated with enhancers, wherein the enhancers may be naturally associated with the particular promoter or associated with a different promoter.

Exemplary strong well-characterized promoters for inducing expression in human T cells include the human immediate early cytomegalovirus (CMV) promoter, a strong ubiquitous classically used viral promoter; murine stem cell virus (MSCV) retroviral LTR promoter, previously described in T cells to mimic transcription regulation of γ-retroviral vector; human phosphoglycerate kinase (PGK), an endogenous housekeeping promoter showed to sustain a moderate and stable expression level; beta-2-microglobulin (β2m) promoter, an ubiquitous and constitutive promoter especially strong in immune cells; human elongation factor 1 alpha (EF1α), a strong ubiquitous constitutive promoter classically used for stable gene transfer, and other promoters such as hPGK and RPBSA promoters. Such promoters may conveniently be used for ex vivo manipulation of T cells, where a constitutive expression of the manipulation is needed, for example in the preparation of ACT compositions. In some embodiments, e.g. for in vivo gene editing, T cell-specific and/or inducible promoters are preferred. In some embodiments, the promoter is a T cell-specific promoter, e.g. a CD3, CD4 or CD8 promoter. Exemplary inducible promoters initiating expression of a gene product upon lymphocyte or T cell activation include NFAT, API, and NR4A promoters.

In another embodiment the construct comprises a first nucleic acid molecule encoding the α chain and having at least 95% identity to the nucleic acid sequence as set forth in SEQ ID NO: 19, and a second nucleic acid molecule encoding the β chain and having at least 95% identity to the nucleic acid sequence as set forth in SEQ ID NO: 20, wherein the encoded TCR is capable of specific binding to an HLA-A2-presented epitope comprising the amino acid sequence of SEQ ID NO: 21 with high functional avidity. In another embodiment, said construct comprises nucleic acid molecules encoding the engineered TCRs of the invention. According to particular embodiments, said first and second nucleic acid sequences comprised in the construct exhibit a high degree of homology (e.g. at least 95%, 96%, 97%, 98% or 99% sequence identity and/or homology to SEQ ID NOs: 19 or 20, respectively), wherein the resulting TCR retains the structural and functional properties as disclosed herein. In another embodiment the encoded TCR is capable of being stably expressed on both CD4⁺ and CD8⁺ cells. In another embodiment the construct is capable of providing an enhanced level and/or duration of expression of the encoded TCR, as compared to a construct encoding a naturally-occurring TCR directed to an HLA-A2-presented epitope as set forth in SEQ ID NO: 21.

In another embodiment the first nucleic acid molecule and the second nucleic acid molecule are connected by a third nucleic acid molecule encoding a linker. In another embodiment, the linker is a cleavable linker. In another embodiment, said linker is a self-cleaving linker. As used herein, the term “cleavable linker” refers to a linker, typically a peptide linker (e.g., about 5-30 amino acids in length) that can be incorporated into multicistronic mRNA constructs such that equimolar levels of multiple genes can be produced from the same mRNA. Non-limiting examples of cleavable linkers include self-cleaving linkers that may be used to generate multiple gene products from a single transcript during translation (typically by ribosomal skipping), and enzymatically cleavable linkers (e.g. furin-cleavable linkers).

In one embodiment, the self-cleaving linker comprises a 2A peptide. In another embodiment, the linker is a 2A peptide (e.g. T2A, P2A, E2A and/or F2A). 2A peptides are typically 18-22 amino-acid (aa)-long viral oligopeptides that mediate “cleavage” of polypeptides during translation in eukaryotic cells. The designation “2A” refers to a specific region of the viral genome and different viral 2A peptides have generally been named after the virus they were derived from. For example F2A is a 2A peptide derived from Foot-and-mouth disease virus (FMDV), E2A is derived from Equine rhinitis A virus (ERAV), P2A is derived from Porcine teschovirus (PTV-1), and T2A is derived from Thosea asigna virus (TaV). In the present specification, the term 2A peptide encompasses viral 2A peptides and 2A-like self-cleaving peptide known in the art. 2A peptide-like sequences are present in various groups of positive- and double-stranded RNA viruses including Picornaviridae, Flaviviridae, Tetraviridae, Dicistroviridae, Reoviridae and Totiviridae. In addition, 2A-like peptides for use in accordance with the invention may include a viral 2A peptide fused to an additional sequence such as an enzyme cleavage site as disclosed herein. In a particular embodiment, said 2A peptide is a P2A peptide having the amino acid sequence ATNFSLLKQAGDVEENPGP (SEQ ID NO: 28).

In various embodiments, the 2A linker may be used with or without a furin recognition site. Furin is a ubiquitously expressed protease that resides in the trans-golgi and processes protein precursors before their secretion. Furin cleaves at the COOH-terminus of its consensus recognition sequence. Various furin consensus recognition sequences (also referred to herein as “furin recognition sites” or “furin cleavage sites”) are known to those of skill in the art. For example, the Arg-X-Lys/Arg-Arg motif can be cleaved by furin enzyme.

In a particular embodiment, said linker is selected from the group consisting of T2A, P2A, E2A and F2A comprising added upstream furin recognition sites. In a particular embodiment, said linker is a P2A linker (with or without said furin recognition site). In another embodiment, said linker may further comprise a “GSG” (Gly-Ser-Gly) linker, e.g. on the N-terminal of a 2A peptide and/or between the 2A peptide and the furin recognition site. According to a preferable embodiment, the linker contains a P2A peptide and the furin recognition sequence Arg-Ala-Lys-Arg, e.g. a linker encoded by SEQ ID NO: 24. In another embodiment the encoded linker has the amino acid sequence RAKRSGSGATNFSLLKQAGDVEENPGPR (SEQ ID NO: 29).

Alternatively, the nucleic acid construct may comprise an internal ribosome entry site (IRES) sequence between the nucleic acid molecules encoding the two chains. As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunoglobulin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). Those of skill in the art would be able to select the appropriate IRES for use in the present invention. Each possibility represents a separate embodiment of the invention.

In another embodiment, there is provided a system comprising: (i) a first construct comprising a first nucleic acid molecule encoding a TCR α chain operatively linked to one or more transcription control elements, and (ii) a second construct comprising a construct nucleic acid molecule encoding a TCR β chain operatively linked to one or more transcription control elements.

In another embodiment, the invention provides an expression vector comprising at least one nucleic acid construct as disclosed herein. The term “expression vector” as used herein, refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include suitable vectors known in the art, such as cosmids, plasmids (e.g., naked or contained in liposomes) and viruses (e.g., lentiviruses, retroviruses, adenoviruses, and adeno-associated viruses) that incorporate the recombinant polynucleotide. In another embodiment, the vector is capable of expressing a TCR of the invention in a mammalian cell. In another embodiment the cell is a human cell (e.g. leukocyte, lymphocyte and/or T cell). In another embodiment, the vector is a viral vector. In a particular embodiment, said vector is a retroviral vector.

Suitable viral vectors are known in the art and available commercially. Exemplary retroviral vectors include, without limitation, pMSGV1, pMSCV, pBMN, pQXIX, pBullet, pBabe, pMSGV, pRETRO, pMIGR, pMX, and pRET. Exemplary lentiviral vectors include, without limitation, pRRL, pCLX, pLenti, pHR, pLVX, and pCAG. Additional exemplary vectors include without limitation, pBullet, pLSC, and adeno-associated viral (AAV) vectors including, but not limited to pAAV and pX601. In another embodiment, said vector is a retroviral vector comprising LTRs from the murine stem cell virus (MSCV LTRs), e.g. a pMSGV1 or pMSCV-derived vectors.

By means of a non-limiting example, a vector of the invention may include a lentiviral vector comprising MSCV LTRs, operatively linked to nucleic acid sequences encoding the α and β chains as set forth in SEQ ID NOs: 19 and 20, respectively, connected via a P2A linker comprising a 5′ furin recognition site.

In another embodiment, the invention provides a host cell comprising the vector. The term “host cell” denotes a cell comprising an exogenous nucleic acid of interest, and is intended to include any individual cell or cell culture that can be or has/have been recipients of a nucleic acid molecule of the invention or a construct or vector encoding same, as well as the progeny thereof, as long as the nucleic acid molecule is present. Suitable host cells include prokaryotic or eukaryotic cells, and also include but are not limited to bacteria, yeast cells, and animal cells such as insect cells and mammalian cells, e.g., murine, rat, or human. According to specific embodiments, the host cell is an immune cell, in particular human immune cells including, but not limited to PBMC or T cell compositions. In another embodiment the host cell is a T cell-containing population, such as human primary lymphocytes or cell populations derived therefrom.

In another embodiment, there is provided a pharmaceutical composition comprising the vector. For example, the pharmaceutical composition may comprise an expression vector or system as disclosed herein as an active ingredient, and optionally one or more pharmaceutically acceptable carriers, excipients or diluents. In another example, said pharmaceutical composition may comprise a host cell (e.g. T cell) comprising said construct or system, and optionally one or more pharmaceutically acceptable carriers, excipients or diluents.

In accordance with this invention, the term “pharmaceutical composition” relates to a composition for administration to a patient, preferably a human patient. Pharmaceutical compositions of the invention comprise a therapeutically effective amount of an active ingredient as disclosed herein (e.g. a nucleic acid construct or a vector, viral particle or cell comprising said nucleic acid construct) and at least one pharmaceutically acceptable excipient. In some embodiments, the pharmaceutical composition comprises a composition for parenteral, transdermal, intraluminal, intravenous, intra-arterial, or intrathecal administration or by direct injection into the tissue or tumor. It is in particular envisaged that said pharmaceutical composition is administered to a patient via infusion or injection. In other embodiments, said composition is formulated for ex-vivo administration into a recipient cell (e.g. derived from PBMC of the subject or suitable donor) which is engineered ex vivo by said composition and re-introduced into the subject.

An “effective amount” or “therapeutically effective amount” refers to an amount sufficient to exert a beneficial outcome in a method of the invention. More specifically, a therapeutically effective amount means an amount of active ingredients effective to prevent, alleviate, or ameliorate symptoms of a disorder (e.g., cancer) or prolong the survival of the subject being treated.

Determination of a therapeutically effective amount is well within the capability of those skilled in the art, especially in light of the detailed disclosure provided herein. For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition (See, e.g., Fingl, E. et al. (1975), “The Pharmacological Basis of Therapeutics,” Ch. 1, p. 1.).

For example, without limitation, nucleic acid constructs in accordance with the invention (e.g. viral vectors) may be used at doses of 1-10 μg/ml for ex-vivo administration, and at doses of 1-300 or 30-100 μg for an adult patient or of 1×10¹⁰-5×10¹⁰ infective units (IU) per patient.

Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well-known conventional methods.

In another embodiment the carrier is a targeting carrier, also referred to herein as a delivery vehicle or an in vivo delivery system. Examples of delivery vehicles particularly suitable for administering nucleic acid agents include, but are not limited to, artificial and natural lipid-containing delivery vehicles. Natural lipid-containing delivery vehicles include exosomes and cellular membranes. Artificial lipid-containing delivery vehicles include liposomes and micelles. Other suitable delivery vehicles include for example nanoparticle-based in vivo delivery systems. A delivery vehicle of the present invention can be modified to target to a particular site in a subject, thereby targeting and making use of a nucleic acid molecule of the present invention at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. For example, compounds that may be used for targeting T cells include e.g. antibodies (or antigen-binding portions thereof) specific to CD3, CD4 and/or CD8.

The compositions of the invention may be administered locally or systematically. Administration will generally be parenterally, e.g., intravenously; DNA may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. It is envisaged that the pharmaceutical composition of the invention might comprise, in addition to the active ingredient as disclosed herein, additional active agents, depending on the intended use of the pharmaceutical composition. Such agents might be anti-cancer drugs, e.g. immunotherapeutic agents such as immune checkpoint inhibitors. The term “checkpoint inhibitor” refers to drugs (e.g. antibodies or small molecules) that target and antagonize, neutralize, or otherwise reduce the activity of immune inhibitory checkpoint molecules like PD-1, PD-L1, and CTLA-4.

In another embodiment, the pharmaceutical composition is amenable for cancer immunotherapy. In another embodiment, the pharmaceutical composition is amenable for use in adoptive transfer therapy (ACT).

### Cell Compositions and Adoptive Cell Transfer Therapy

In another aspect, the invention relates to a cell composition expressing a TCR of the invention. In another embodiment, there is provided a cell composition for cancer immunotherapy, characterized by surface expression of a TCR of the invention. In another embodiment, the cell composition is an ACT composition. Thus, a cell composition expressing (or characterized by surface expression of) a TCR of the invention comprise at least one population of cells expressing said TCR. In some embodiments, there is provided a cell composition for cancer immunotherapy, comprising a therapeutically effective amount of immune cells engineered to express a TCR directed to NY-ESO-1, the TCR comprising:
(a) a TCR α chain comprising: i.a VR comprising a CDR1 having the amino acid sequence of SEQ ID NO: 1, a CDR2 having the amino acid sequence of SEQ ID NO: 2, and a CDR3 having the amino acid sequence of SEQ ID NO: 3; ii. a CR comprising a cysteine residue at position 47 thereof and the amino acid sequence as set forth in SEQ ID NO: 22 at positions 250-254 thereof;
(b) a TCR β chain comprising: i. a VR comprising a CDR1 having the amino acid sequence of SEQ ID NO: 4, a CDR2 having the amino acid sequence of SEQ ID NO: 5, and a CDR3 having the amino acid sequence of SEQ ID NO: 6; and ii. a CR comprising a cysteine residue at position 57 thereof; and
(c) a plurality of interchain disulfide bonds between said α chain and β chain.

In another embodiment, the TCR is characterized as disclosed herein. The terms “engineered” and “genetically engineered” may be used interchangeably.

The term “cell composition” as used herein indicates a pharmaceutical composition that contains cells or cellular material as the active ingredient. Cell compositions typically contain pharmaceutically acceptable carriers, excipients or diluents, and optionally additional components other than cells such as culture medium or preservation liquid. The term “cancer immunotherapy” refers to treatment of a subject afflicted with, or at risk of suffering a recurrence of cancer, by a method comprising modulating an immune response in the subject. In particular, cancer immunotherapies are typically aimed at inducing and/or stimulating the immune response of the subject towards cancer cells. Exemplary agents and regimes used in cancer immunotherapy include immune checkpoint modulators (such as checkpoint inhibitors or agonists), tumor-specific antibodies, cytokines and therapeutic anti-cancer vaccines. Another common form of cancer immunotherapy involves the adoptive transfer of immune cells (ACT), in particular of effector cells including T cells, NK cells and/or NK T cells.

As used herein, and unless otherwise specified, the term “adoptive transfer” refers to a form of passive immunotherapy where previously sensitized immunologic agents (e.g., cells or serum) are transferred to the recipients. The phrases “adoptive cell transfer”, “adoptive transfer immunotherapy”, “adoptive transfer therapy”, “adoptive cell therapy” and “adoptive cell immunotherapy” are used interchangeably herein to denote a therapeutic or prophylactic regimen or modality, in which effector immunocompetent cells are administered (adoptively transferred) to a subject in need thereof, to alleviate or ameliorate the development or symptoms of cancer or infectious diseases.

The adoptively transferred cells are often directed to a tumor or a tumor-associated antigen, for example the ACT compositions of the invention are directed to NY-ESO-1. In particular ACT compositions of the invention contain an effective amount of immune cells (typically comprising T cells) engineered to express a TCR of the invention, as disclosed herein. Thus, an ACT composition in accordance of the invention contains effective amounts of engineered cells as disclosed herein, which are produced under sterile and suitable (e.g. cGMP grade) conditions, to be administered to a human subject afflicted with a neoplastic disorder as part of their anti-tumor regimen.

As used herein in connection with cell compositions, an effective amount (also referred to herein as a therapeutically effective amount) is an amount sufficient to induce or enhance a beneficial immune response such as an anti-tumor response when administered to a subject, e.g. 10⁶ to 10¹² cells or 10⁷ to 10¹¹ cells. In particular, with respect to the ACT compositions of the invention, the effective amount is an amount sufficient to induce or enhance said immune response upon adoptive transfer to a subject in need thereof, and typically comprises at least 10⁹ and more typically at least 5×10⁹ cells, and up to about 10×10¹⁰ cells or more, e.g. 5×10⁹-10×10⁹ viable cells provided by a preparation process as disclosed herein.

T lymphocytes (T cells) are one of a variety of distinct cell types involved in an immune response. The activity of T cells is regulated by antigen, presented to a T cell in the context of a major histocompatibility complex (MHC) molecule. The T cell receptor (TCR) then binds to the MHC-antigen complex. Once antigen is complexed to MHC, the MHC-antigen complex is bound by a specific TCR on a T cell, thereby altering the activity of that T cell. Proper activation of T lymphocytes by antigen-presenting cells requires stimulation not only of the TCR, but the combined and coordinated engagement of its co-receptors.

T cells (for example α/β T cells) are typically characterized by expression of CD3, and are thus generally considered CD3⁺ cells. CD3 is the TCR co-receptor consisting of four transmembrane subunits (γ, δ, ε, and ζ), functioning as and involved in TCR-mediated activation. In particular, the intracellular portions of the CD3 γ, δ, ε, and ζ subunits contain copies of a sequence motif termed ITAMs (immunoreceptor tyrosine-based activation motifs) facilitating TCR-mediated signaling. Thus, a TCR of the invention may optionally be engineered to be expressed in an immune cell lacking endogenous CD3 expression, by engineering said cells to co-express CD3 (in addition to the TCR of the invention), e.g. by recombinant methods as disclosed herein.

Memory (primed) T cells include for example central memory cells (T_{CM}) expressing CCR7 but not CD45RA, effector memory (T_{EM}) cells lacking expression of both CCR7 and CD45RA, and/or T_{EMRA} cells that express CD45RA but lack expression of CCR7.

T helper cells (TH cells) assist other white blood cells in immunologic processes, including maturation of B cells into plasma cells and memory B cells, and activation of cytotoxic T cells and macrophages. These cells are also considered CD4⁺ T cells because they express the CD4 glycoprotein on their surfaces. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. CD4 is complexed with the bound TCR and is considered to be involved in stabilization of MHC II-restricted TCR complexes.

Cytotoxic T cells (TC cells, or CTLs) destroy virus-infected cells and tumor cells, and are also implicated in transplant rejection. These cells are also considered CD8⁺ T cells since they express the CD8 glycoprotein at their surfaces. These cells recognize their targets by binding to antigen associated with MHC class I molecules, which are present on the surface of all nucleated cells. CD8 is complexed with the bound TCR and is considered to be involved in stabilization of MHC I-restricted TCR complexes.

However, while “CD4⁺ cells” and “CD8⁺ cells” are often used to refer to TH cells and TC cells, respectively, it is noted that additional CD3⁺ lymphocyte populations may be present in the cell compositions of the invention. For example, CD4⁺ CTLs are effector cells that possess cytotoxic function in the absence of CD8 expression. According to embodiments of the present disclosure, cell compositions of the invention are characterized by a unique profile of sub-populations distinct from other hitherto disclosed ACT compositions.

The invention in embodiments thereof provides for the production of ACT compositions in which greater than 50% of the cells are characterized by surface expression of the TCR, and typically to an average expression greater than 53%, 55%, 60%, 70%, 80%, 90% or more, as disclosed herein. As a CPN<5 is recommended for regulatory purposes, enhancing the TCR surface expression per copy number is advantageous. The invention in embodiments thereof provides for the production of ACT compositions adapted for clinical use, characterized CPN<5 and by enhanced TCR surface expression as disclosed herein. Further, compositions characterized by high eTCR expression as disclosed herein can be produced not only when using healthy donor allogeneic immune cells, but also when using autologous cells from cancer patients (which are often be immune suppressed or compromised, due to the presence of the tumor (tumor-derived immune suppression) and/or to prior treatment by other cancer therapies (such as chemotherapy or irradiation that may induce depletion or suppression of immune cells).

In another embodiment, said composition comprise both CD4⁺ eTCR⁺ cells and CD8⁺ eTCR⁺ cells. In another embodiment said composition comprises a CD8⁻ effector cell population. In another embodiment said composition comprises a cell population characterized by surface expression of CD4, a TCR of the invention of and CD107 (CD4⁺ eTCR⁺ CD107⁺ cells). According to exemplary embodiments, an ACT composition of the invention may comprise a CD4⁺ eTCR⁺CD107⁺ cell population constituting 5-40%, 10-30%, 10-20%, 10-25%, 15-30%, e.g. about 15% out of the total CD4⁺ eTCR⁺ cells in the composition. Further, ACT compositions in accordance with the invention exhibit functional properties as disclosed herein with respect to the activity of the engineered TCR.

In another embodiment, at least 60%, 70%, 80% or 90% of the immune cells are characterized by surface expression of said engineered TCR (eTCR⁺ cells). In another embodiment, at least 70% of the immune cells are CD3⁺ eTCR⁺ cells and comprise both CD4⁺ and CD8⁺ cells. In another embodiment the composition comprises 20-50%, 15-50%, 15-40%, 40-50%, 20-60% or 20-40% CD4⁺ cells and 30-70%, 30-85%, 40-50%, 40-85%, 50-70% or 50-85% CD8⁺ cells, wherein each possibility represents a separate embodiment of the invention. As disclosed herein, the CD4⁺ cells in the compositions of embodiments of the invention are functionally reactive against NY-ESO-1—expressing tumor cells. Additionally, or alternatively, said immune cells were engineered to express said TCR by administration of a nucleic acid construct encoding said TCR, and said cell composition is characterized by an average copy number of five or less construct molecules per cell.

In another embodiment, the composition may further comprise an additional active ingredient, e.g. an anti-cancer drug or a cytokine. In a particular embodiment, the anti-cancer drug is a cancer immunotherapy, e.g. an immune checkpoint inhibitor. In another particular embodiment, the cytokine is a T cell-modulating cytokines, e.g. interleukin-2 (IL-2). In another embodiment, the composition further comprises IL-2 (e.g. 100-500 IU/mL). In another embodiment said composition comprises at least 10⁹ CD3⁺ eTCR⁺ cells, e.g., 10⁹-5×10¹⁰, 5×10⁹-10¹⁰, or 10⁹-10¹⁰ cells.

By means of a non-limiting example, a clinical-grade cell composition for ACT may include at least 10⁹ autologous CD3⁺ eTCR⁺ cells suspended in injection medium, comprising 0.9% Sodium Chloride, 2% Human Albumin and IL-2 300 IU/mL in a total volume of 300 mL.

In another embodiment, the cell composition is prepared by a process as disclosed herein. In another embodiment, there is provided a process for preparing a cell composition as disclosed herein.

In another embodiment, the processes of the invention comprise the steps of:
a. providing an immune cell population, typically a T-cell containing cell population,
b. culturing the cell population in the presence of a CD3-specific antibody and IL-2,
c. engineering the cells resulting from step b. to express a TCR of the invention,
d. expanding the cells resulting from step c. so as to obtain a therapeutically effective amount of expanded engineered cells, and
e. harvesting the expanded engineered cells resulting from step d.

Step a. is further referred to herein as the sample obtaining step. In another embodiment the T-cell containing cell population provided in step a. is a peripheral blood mononuclear cells (PBMC) population. In another embodiment, the cell population is obtained by apheresis (e.g., leukapheresis). In another embodiment, the cells may be subjected to additional steps of enrichment or isolation of specific cell population. In another embodiment, the cell populations may include e.g. isolated T cells, memory T cells, or T cell lines. In another embodiment said T cell-containing cell population comprises or is derived from a tumor-infiltrating lymphocyte population. In another embodiment, said PBMC population is provided in the form of a non-fractionated PBMC sample. Each possibility represents a separate embodiment of the invention. In another embodiment, the PBMC population has been cryopreserved and thawed prior to step b.

Peripheral blood mononuclear cells (PBMC or PBMCs) are leukocytes isolated from peripheral blood and comprise cells having round nuclei such as lymphocytes, monocytes, and dendritic cells. Typically, lymphocytes constitute the majority of the PBMC population. PBMCs may be extracted from whole blood using methods known in the art, for example using Ficoll-hypaque density gradient centrifugation. Additionally or alternatively, PBMC or specific leukocyte populations thereof may also be isolated from peripheral blood using apheresis. The apheresis product may contain lymphocytes, including T cells, monocytes, granulocytes, B cells, other nucleated white blood cells, red blood cells, and platelets or may be a leukapheresis product comprising lymphocytes, including T cells, monocytes, granulocytes, B cells, and other nucleated white blood cells. In one embodiment, the cells collected by apheresis may be washed to remove the plasma fraction and to place the cells in an appropriate buffer or media for subsequent processing (e.g. density gradient or additional purification steps). The cells can be washed with PBS or with another suitable solution that lacks calcium, magnesium, and most, if not all other, divalent cations. As used herein, and unless indicated otherwise, the term PBMC is further used to denote in particular human PBMC. Non-limiting examples of PBMC isolation are provided in the Examples below.

Step b. is also referred to herein as the activation (or stimulation) step, in which the cells provided in step a. are cultured in the presence of a CD3-specific antibody and IL-2 so as to induce T cell activation. The term “activation,” as used herein, refers to the state of an immune cell that has been sufficiently stimulated to induce detectable cellular proliferation. Activation can also be associated with induced cytokine production, and detectable effector functions. The term “activated T cells” refers to, among other things, T cells that are undergoing cell division.

In another embodiment, step b. further comprises culturing said cells with a CD28-specific antibody, wherein the CD3-specific antibody and the CD28-specific antibody are surface-bound. For example, without limitation, the antibodies may be bound to the surface of particles, beads, nanoparticles, culture dishes and the like. In another embodiment, step b. may be performed for 1-3 days, e.g. 2 days. CD28 refers to the protein Cluster of Differentiation 28, one of the proteins expressed on T cells that provide co-stimulatory signals required for T cell activation and survival. CD28 is the receptor for CD80 and CD86 proteins A sequence of human CD28 is provided in NCBI Reference No: NP_006130. In this specification and claims, “anti-CD28 antibody” and “anti-CD3 antibody” refer to anti-CD28 agonist antibody and anti-CD3 agonist antibody, respectively, namely to antibodies that specifically stimulate CD28 receptors and CD3 receptors on cells, respectively.

The term “antibody” is used in the broadest sense and includes monoclonal antibodies (including full length or intact monoclonal antibodies), polyclonal antibodies, multivalent antibodies, and antibody fragments long enough to exhibit the desired biological activity, e.g. specific binding to CD3 or CD28 (in particular, to human CD3 or CD28) in an agonistic manner. Methods of generating monoclonal and polyclonal antibodies are well known in the art. Antibodies may be generated via any one of several known methods, which may employ induction of in vivo production of antibody molecules, screening of immunoglobulin libraries, or generation of monoclonal antibody molecules by continuous cell lines in culture. These include, but are not limited to, the hybridoma technique, the human B-cell hybridoma technique, and the Epstein-Barr virus (EBV)-hybridoma technique. Besides the conventional method of raising antibodies in vivo, antibodies can be generated in vitro using phage display technology, by methods well known in the art (e.g. Current Protocols in Immunology, Colligan et al (Eds.), John Wiley & Sons, Inc. (1992-2000), Chapter 17, Section 17.1). In addition, various antibodies specific to human CD3 and CD28 are commercially available, for example agonistic anti-CD3 antibodies include OKT3 (ATCC CRL-8001/Invitrogen), UCHT1 (B. D. PharMingen), and HIT3a (B. D. PharMingen), and agonistic anti-CD28 antibodies include e.g. CD28.2 (Biologend). Anti-CD3 and anti-CD28 antibodies provided in a surface-bound form amenable for use in embodiments of the invention is available from Thermo Scientific and Milteny, as exemplified in the Examples below.

Step c. is also referred to herein as the engineering step, and comprises genetically engineering the activated cells resulting from step b. to express a TCR of the invention. Engineering may be performed by suitable methods known in the art such as transduction, infection, transfection or transformation, such that it is detectable on the surface of at least a portion of the resulting cell population by conventional methods such as flow cytometry. In another embodiment step c. is performed by incubating the cells following step b. with a viral vector comprising a nucleic acid construct encoding said TCR (so as to transduce said cells with said construct). In some embodiments, fibronectin fragments (e.g. Retronectin) or other agents promoting co-localization of a viral vector and the intended target cells may be used to improve transduction efficacy.

Step d. is also referred to herein as the expansion step, which includes further culturing of the engineered cells under specific conditions such that the total number of eTCR⁺ cells of a desired phenotype is enhanced. For example, expansion may employ the use of T-cell modulating cytokines that induce T cell proliferation such as IL-2, typically in combination with other T cell-stimulating agents as disclosed herein. In another embodiment, step d. is performed by incubating the cells following step c. with IL-2 and optionally feeder cells (e.g., attenuated allogeneic cells such as irradiated PBMC). In another embodiment step d. is performed in the absence of supplementation of antibodies. In another embodiment step d. is performed so as to obtain at least 10⁹ CD3Y eTCR⁺ cells. In another embodiment step e. is performed within 7-21, typically 14-21 days of initiating step b. In some embodiments, step e. is performed within 20, 19, 18, 17, 16, 15 or 14 days of initiating step b., wherein each possibility represents a separate embodiment of the invention. In a particular embodiment step e. is performed within 14 to 15 days of initiating step b. In other particular embodiments, step e. is performed within 13, 12, 11, 10, 9, 8 or 7 days of initiating step b., wherein each possibility represents a separate embodiment of the invention.

The term “feeder cells” generally refers to cells of one type that are co-cultured with cells of a second type, to provide an environment in which the cells of the second type can be maintained and proliferated. For the purpose of the present invention, this term specifically refers to Fc receptor-bearing accessory cells, which are typically allo-reactive with the T cell containing population to be propagated. In other words, the feeder cells need not be histocompatible with the T-cell containing population to be propagated, and in certain advantageous embodiments the two populations typically HLA-mismatched. A typical example of feeder cells used in embodiments of the invention is allogeneic normal donor peripheral blood mononuclear cells, PBMC. For example, without limitation, ACT compositions may be prepared using irradiated PBMC (incapable of proliferation) as feeder cells, e.g. attenuated by irradiation by exposing the cells to 6000RAD.

Step e. is also referred to herein as the harvesting step. This step comprises separating and collecting cells from their culture medium and preparing the cells for therapeutic applications. Harvesting typically comprises washing cells with a suitable buffer (e.g. a BSA-containing saline solution), and optionally resuspending cells in a buffer that is suitable for intended applications, e.g., for infusion (for example, suspension in 100-400 ml of in a saline solution with 1-5% human albumin and 100-500 IU/mL IL-2, e.g. in 200-400 ml of a 0.9% saline solution with 2% human albumin and 300 IU/mL IL-2).

Determining the therapeutically effective amount of expanded engineered cells obtained and harvested by the methods of the invention is well within the knowledge of the skilled artisan. For example, the total number of viable cells may be calculated by staining a sample of the cells with a viability dye (e.g. typan blue) and counting the cells using a hemocytometer. The number of CD3⁺ eTCR⁺ cells may be evaluated by flow cytometry (e.g. following staining with suitable antibodies and/or NY-ESO-1-specific tetramers). The effective amount of viable CD3⁺ eTCR⁺ cells obtained may then be calculated by multiplying the percentile of CD3⁺ eTCR⁺ cells by the total number of viable cells.

Thus, advantageous processes in accordance with the invention provide cell compositions with desirable phenotypes, exhibiting enhanced levels of activations markers and reduced levels of exhaustion markers as compared to hitherto reported compositions. In a particular embodiment, said activation markers comprise cytotoxic effector markers (e.g. CD107). According to some embodiment, an ACT composition of the invention is characterized by enhanced levels of activation markers including CD134, CD137 and/or CD107 as compared to hitherto-known compositions.

Suitable conditions for affecting the processes of the invention are provided and exemplified by the present specification, as detailed herein. The present specification demonstrates the implementations of these processes and conditions to provide therapeutically effective amounts of viable CD3⁺ eTCR⁺ cells that are characterized by said advantageous phenotype, amenable for the production of both autologous and allogeneic ACT compositions.

In another embodiment the process comprises the steps of:
a. providing a PBMC population,
b. culturing the cell population in the presence of a CD3-specific antibody and IL-2,
c. engineering the cells (resulting from step b.) to express the TCR by incubating said cells with a viral vector comprising a nucleic acid construct encoding said TCR,
d. expanding the cells following step c. in the presence of IL-2 and feeder cells, and in the absence of supplementation of antibodies, so as to obtain at least 10⁹ CD3⁺ cells expressing said TCR, and
e. harvesting the expanded cells within 7-21 or 14-21 (e.g. 15) days of initiating step b.

In another embodiment, the steps as recited above include the culturing/expansion of the cells in the sole presence of the antibodies, cells and/or and cytokines as recited in steps b. and d. above, and without the addition of other antibodies, cells and/or and cytokines. For example, without limitation, step b. advantageously includes culturing the cell population in the presence of 10-100, e.g. 50 ng/mL of a CD3-specific antibody (e.g. OKT3) and 100-500, e.g. 300 IU/mL IL-2 as the sole activators, and step d. advantageously includes culturing the resulting cells with irradiated allogeneic PBMC at a ratio of PBMC to engineered cells of 25:1 to 100:1 (e.g. 25:1 to 75:1, 40:1 to 75:1, or about 50:1) and 1500-4500, e.g. 3000 IU/mL IL-2, in the absence of supplementation of antibodies or other cytokines. Yet in other embodiments, additional methods for activating and/or expanding the cells may be used, which substantially retain the parameters of the resulting cell composition as disclosed herein. For example, without limitation, a colloidal polymeric nanomatrix covalently attached to humanized recombinant agonists against human CD3 and CD28 (e.g. TRANSACT) may be used in combination with IL-2 in the activation and expansion steps, as detailed herein. In another example, uniform 4.5 μm diameter, inert, superparamagnetic beads similar in size to antigen-presenting cells, and covalently coupled to anti-CD3 and anti-CD28 antibodies (e.g. DYNABEADS) may be used in combination with IL-2 in the activation and expansion steps, as detailed herein. By means of non-limitative examples, surface-bound anti-CD3 and anti-CD28 antibodies may be used at concentration ranges of 0.2 μg/ml to 5 μg/ml.

For instance, the Examples section below demonstrate the use of processes in which sample obtaining is performed by providing an autologous or allogeneic PBMC population; activation is performed by culturing the cell population in the presence of a CD3-specific antibody and IL-2 as the sole activators for 2-3 days under the conditions as described above; engineering is performed by transduction with a with a TCR-encoding viral vector of the invention (e.g. a retroviral vector as disclosed herein) in the presence of surface-bound fibronectin fragments; and expansion is performed by culturing the resulting cells with IL-2 and irradiated allogeneic PBMC as feeder cells, in the absence of supplementation of antibodies or other cytokines, under the conditions as described above for 11-13 days, and the harvested cells are further suspended in saline solution for injection comprising BSA and IL-2. It is herein demonstrated that the process may be used for obtaining at least 10⁹ CD3⁺ eTCR⁺ cells phenotypically characterized as disclosed herein, providing for improved ACT. Other examples for suitable reaction condition (for example when using surface-bound CD3-specific and CD28-specific antibodies) are further provided in the Examples section below.

Further as disclosed herein, ACT compositions of the invention provide for remarkable persistence in blood circulation in tumor-bearing individuals using both animal models and human cancer patients. According to some embodiments, an ACT composition of the invention is detectable in the blood of a recipient subject in need thereof for over 30 days and more typically at least 45, 60, 75 or 90 days or for at least 4, 5, 6, 7 or 8 months and up to about 9 months or more, e.g. 45 days to 9 months, 2-9 months, or 3-9 months in a human subject.

### Therapeutic Use

In another aspect, there is provided a method of treating cancer in a subject in need thereof, comprising administering to the subject an ACT composition of the invention. In various embodiments, the cells are autologous, allogeneic histocompatible, allogeneic partly histocompatible (e.g. substantially histocompatible), or allogeneic non-histocompatible with the subject. Typically and advantageously, the use of autologous and allogeneic histocompatible ACT compositions is contemplated. In a particular embodiment, the cells are autologous to the subject.

The term “autologous” as used herein, refers to any material derived from a subject to which it is later to be re-introduced into the same subject. According to certain preferable embodiments, the cell composition is histocompatible with the subject to be treated (e.g. autologous cells or MHC II-matched allogeneic cells). The term “histocompatibility” refers to the similarity of tissue between different individuals. The level of histocompatibility describes how well matched the patient and donor are. The major histocompatibility determinants are the human leukocyte antigens (HLA). HLA typing is performed between the potential donor and the potential recipient to determine how close an HLA match the two are. The term “histocompatible” as used herein refers to embodiments in which all six of the HLA antigens (2 A antigens, 2 B antigens and 2 DR antigens) are the same between the donor and the recipient. However, in other embodiments, donors and recipients who are “mismatched” at two or more antigens, for example 5 of 6, or in other embodiments, 4 of 6 or 3 of 6 match, may be encompassed by certain embodiments of the invention, despite the donor and recipient not having a complete match. The term “substantially histocompatible” as used herein refers to embodiments in which five out of six of the HLA antigens are the same between the donor and the recipient.

In another aspect, there is provided a method of treating cancer in a subject in need thereof, comprising administering to the subject a pharmaceutical composition comprising a construct of the invention. For example, without limitation, the construct may be delivered to the subject in the form of an expression vector exhibiting cell-type or tissue specificity (e.g. to immune cells as disclosed herein), or in the form of RNA molecules delivered via nanoparticles or other suitable in vivo delivery systems.

For example, without limitation, the methods of the invention may be effected by administering to the subject a pharmaceutical composition comprising a therapeutically effective amount of an expression vector comprising a nucleic acid construct encoding a TCR of the invention, e.g. comprising a first nucleic acid sequence encoding a TCR α chain in which the CDRs are encoded by nucleic acid sequences as set forth in SEQ ID NOs: 7-9, and a second nucleic acid sequence encoding a TCR β chain in which the CDRs are encoded by nucleic acid sequences as set forth in SEQ ID NOs: 10-12. In various exemplary embodiments, the vector exhibits T cell specificity, e.g. using T cell-specific promoters as disclosed herein.

In another aspect, the invention provides a method of enhancing anti-tumor immunity in a subject in need thereof, comprising administering to the subject a cell composition of the invention, or, in other embodiments, a pharmaceutical composition comprising a construct of the invention. Each possibility represents a separate embodiment of the invention. As used herein, enhances anti-tumor immunity comprises inducing or augmenting a tumor-specific immune response (also referred to herein as tumor-specific immune reactivity) in the subject. In another embodiment, the response comprises a T cell mediated eliciting or enhancing a TCR-mediated functional activity (such as cytotoxicity or cytokine production), specifically against an NY-ESO-1-expressing tumor as disclosed herein. In other embodiments, enhancement of anti-tumor immunity comprises enhancement of tumor-specific CD8-independent cytotoxicity. In another embodiment, enhancement of anti-tumor immunity is manifested by a significant tumor-specific immune response for over 30 days and more typically at least 45, 60, 75 or 90 days or for at least 4, 5, 6, 7 or 8 months and up to about 9 months or more, e.g. 45 days to 9 months, 2-9 months, or 3-9 months. In another embodiment, enhancement of anti-tumor immunity comprises diminishing a deficiency associated with a prior or previously-administered adoptive transfer therapy. In another embodiment, the methods of the invention provide for inducing or augmenting a tumor-specific immune response characterized by advantageous functional properties as disclosed herein. Each possibility represents a separate embodiment of the invention.

In some embodiments, methods of the invention employing the use of ACT compositions may typically comprise a step of lymphodepletion prior to adoptively transferring the cells to the recipient subject. Lymphodepletion protocols are well known and readily performed by the treating physician and adjusted to the clinical status and medical history of the patient. For example, without limitation, lymphodepletion may be employed by a non-myoablative lymphodepletion protocol (e.g. using cyclophosphamide, with or without fludarabine) e.g. within 5-6 days prior to ACT administration. Non-limitative examples of such protocols are provided in Example 9.

Typically, in the methods of the invention, the subject is afflicted with a NY-ESO-1 expressing tumor. In addition, the subject to be treated by the methods of the invention is typically HLA-A2-positive. The terms “expressing” or “positive” are well-known in the art and denote that a gene product is expressed in an amount detectable by an approach known in the art. A protein can be detected by use of immunological assay, for example, ELISA, immunostaining, or flow cytometry, e.g. using a labeled antibody. A transcript can be detected by use of a method of amplifying and/or detecting nucleic acid, for example, RT-PCR, microarray, biochip, or RNAseq.

The terms “NY-ESO-1 expressing tumor”, “NY-ESO-1 cancer” and “tumor cell that expresses NY-ESO-1” as used herein refer to a tumor comprising cells that express NY-ESO-1. These cells are typically characterized by surface expression of at least one epitope of a naturally-occurring NY-ESO-1 polypeptide in the context of an HLA molecule, in particular an HLA class I molecule. More typically, the HLA molecule is an HLA-A2 molecule.

HLA-A2 is one particular class I major histocompatibility complex (MHC) allele group at the HLA-A locus; the α chain is encoded by the HLA-A*02 allele group and the β chain is encoded by the β2-microglobulin or B2M locus. The following A2 alleles are known: A*02:01, A*02:02, A*02:03, A*02:04, A*02:05, A*02:06 (A2.4A), A*02:07, A*02:08, A*02:09, A*02:10, A*02:11 (A2.5), A*02:12, A*02:13 (A*02SLU), A*02:16, A*02:17, A*02:18 (A2K), A*02:19, A*02:20, A*02:21, A*02:31, A*02:34 (A*AAT), A*02:35, A*02:36 and A*02:37. The most common allele is the A*02:01 allele, and thus most subjects identified as having HLA-A2 bear the A*02:01 allele (also referred to as HLA-A*201 or HLA-A2*01).

In some embodiments, the tumor is selected from the group consisting of: melanoma, bladder, ovarian, lung, breast, and prostate tumors (typically a NY-ESO-1 expressing tumors). In other embodiments, the subject to be treated by the methods of the invention may be afflicted with e.g. neuroblastoma, myeloma, metastatic melanoma, synovial sarcoma, bladder cancer, esophageal cancer, hepatocellular cancer, head and neck cancer, non-small cell lung cancer, ovarian cancer, prostate cancer, or breast cancer. In yet other embodiments, the tumor may be e.g. a melanoma (e.g. uveal melanoma), an ovarian tumor (e.g. ovarian carcinoma), a synovial tumor (e.g. synovial sarcoma) or a breast tumor (e.g. triple-negative breast cancer, TNBC). According to exemplary embodiments, the tumor may be e.g. a NY-ESO-1 expressing tumor selected from uveal melanoma, synovial sarcoma, TNBC, and ovarian carcinoma. In other particular embodiments, the subject is afflicted with a tumor selected from the group consisting of melanoma (e.g. uveal melanoma), sarcoma (e.g. synovial sarcoma), and carcinoma (e.g. bladder, ovarian, lung, breast, and prostate carcinoma). In yet another embodiment the tumor may be a NY-ESO-1 expressing melanoma, bladder, ovarian, lung, breast, prostate or synovial tumor. In another embodiment said tumor is selected from the group consisting of: melanoma, myeloma, bladder, ovarian, lung, breast, synovial and prostate tumors. Each possibility represents a separate embodiment of the invention.

In another embodiment, the methods of the invention further comprise a step for determining expression of HLA-2 and/or NY-ESO-1 in a sample of a subject prior to treatment (for example, determining whether the subject is afflicted with a tumor expressing an HLA-2-presented NY-ESO-1 epitope) e.g. by an immunoassay or amplification method as disclosed herein. Thus, according to some embodiments, a subject determined to be afflicted with an HLA-2 and NY-ESO-1 positive tumor is treated by a pharmaceutical composition or cell composition of the invention. According to particular embodiments, a tumor to be treated by the methods of the invention expresses a peptide epitope of SEQ ID NO: 21 in the context of HLA-A*0201 and/or HLA-A*0206.

In another embodiment, the tumor is a sloid tumor. In another embodiment, said tumor is metastatic. In another embodiment, said tumor is a locally advanced tumor. In another embodiment, said tumor is a refractory (treatment resistant) tumor. In another embodiment, said tumor is a recurrent tumor or malignancy. In yet another embodiment, said tumor is a metastatic cancer or a locally advanced refractory or recurrent malignancy, not otherwise amenable to curative treatment.

In another embodiment, the patient had received prior treatment with a cancer therapy such as chemotherapy and/or immunotherapy. In another embodiment said patient had failed to respond to at least one course of treatment by chemotherapy and/or immunotherapy. In another embodiment, said patient is afflicted with a tumor resistant to said treatment. In another embodiment, said treatment is immunotherapy by immune checkpoint inhibitors. In another embodiment said checkpoint inhibitors are directed to PD-1 and/or CTLA-4. In another embodiment, said patient had received and failed to respond to prior treatment with immune checkpoint inhibitors directed to PD-1 and/or CTLA-4. Each possibility represents a separate embodiment of the invention.

The term “resistance” refers to the feature of cancer (or tumor) not responding to a given treatment. A tumor can be resistant to a particular therapy already at the initiation of treatment (primary resistance). Alternatively, a tumor can develop resistance during the course of treatment (acquired resistance, also referred to as secondary resistance). The resistance to therapy is commonly understood as unsatisfactory effectiveness of treatment usually resulting in disease progression. In the case of acquired resistance, the resistance can also be manifested as a decrease in the amount of tumor regression at the same dose or an increase in the dose necessary for the same amount of tumor regression. For example, in the case of resistance to immune checkpoint inhibitors, the term “resistant” refers to a tumor that fails, or has failed, to respond adequately in a favorable manner in terms of tumor shrinkage or duration of stabilization or shrinkage in response to treatment with an immune checkpoint inhibitor for a time of greater than 3 months or more.

The term “amenable for treatment” refers to a tumor predicted or determined to respond favorably to the treatment. Amenability for treatment can be determined by the treating physician or oncologist by assessing clinical response to the treatment (or to a similarly acting treatment of the same category) or predicted by the skilled artisan by assessing cell viability or apoptosis-inducing activity after bringing a suitable anti-cancer agent in contact with a tumor or tumor sample originating from a subject. The skilled person is aware of the existence of standard assays to determine treatment amenability or screen for resistant cancers, such as MTT assays, ATP-measurements and/or apoptosis-assays such as TUNEL, Cytochrome C release or Cleaved Caspase-3 assays. In another embodiment the methods of the invention further comprise administering to the subject a cancer immunotherapy. In another embodiment, said immunotherapy is administered prior to, concurrently with, or after adoptive transfer of an ACT composition of the invention, wherein each possibility represents a separate embodiment of the invention. In a particular embodiment the cancer immunotherapy is a T-cell mediated immunotherapy (directed at inducing, enhancing or otherwise modulating the activity of T cells in the subject). Exemplary T-cell mediated immunotherapies include, but are not limited to, T cell-modulating cytokines, immune checkpoint inhibitors and cell composition immunotherapies (e.g. adoptive transfer cell compositions) as disclosed herein.

For example, without limitation, the subject is further treated by one or more cytokines (e.g. IL-2, IL-7, IL-12, IL-15, and IL-21). In other exemplary embodiments, co-treatment with immune checkpoint inhibitors (e.g. directed to PD-1, CTLA4, TIGIT, TIM3, PD-L1, 41BB, FAS, GITR, BTLA, ICOS, OX40, LAG-3, GLECTIN 9, CD96, VISTA, H-VEM, P-SELECTIN, or PCGL), is contemplated. In another embodiment, said tumor is resistant to treatment by one or more of the above-mentioned cytokines or immune checkpoint inhibitors, wherein each possibility represents a separate embodiment of the invention. In another embodiment, said tumor is an immunotherapy resistant tumor, not amenable for treatment with immunotherapies such as cytokines and immune checkpoint inhibitors.

The following examples are presented in order to more fully illustrate some embodiments of the invention. They should, in no way be construed, however, as limiting the broad scope of the invention.

### EXAMPLES

### Example 1. Generation of Novel Engineered TCR Directed to NY-ESO-1

A novel engineered TCR that recognizes NY-ESO-1 in the context of the HLA-A*0201 class I restriction element was constructed. The TCR construct encodes the human α and β chains having the amino acid sequence as set forth in SEQ ID NOs: 17 and 18, respectively. The construct contains the nucleic acid sequences encoding the α and β chains as set forth in SEQ ID NOs: 19 and 20, respectively, connected via a P2A linker comprising a 5′ furin recognition site, having the amino acid as set forth in SEQ ID NO: 29 and the nucleic acid sequence as set forth in SEQ ID NO: 24, as follows:

(SEQ ID NO: 24)
AGGGCAAAGCGCAGCGGATCCGGAGCAACAAACTTTAGCCTGCTGAAGCA
GGCAGGCGACGTGGAGGAGAATCCAGGACCGCGG.

Thus, the sequence of the construct encoding the linked α and β chains has the nucleic acid sequence as set forth in SEQ ID NO: 25, as follows (in which the signal peptides are underlined and the linker is presented in bold):

(SEQ ID NO: 25)
ATGTTCGAAACTCTGCTGGGGCTGCTGATTCTGTGGCTGCAGCTGCAGTG
GGTGTCATCCAAACAGGAGGTCACTCAGATTCCCGCTGCCCTGAGCGTGC
CTGAGGGCGAGAACCTGGTGCTGAATTGCTCCTTCACCGACTCTGCCATC
TACAACCTGCAGTGGTTTAGGCAGGATCCAGGCAAGGGCCTGACCAGCCT
GCTGCTGATCCAGAGCTCGCAGAGGGAGCAGACATCCGGCCGCCTGAATG
CCTCTCTGGACAAGTCTAGCGGCCGGAGCACCCTGTACATCGCAGCAAGC
CAGCCAGGCGATTCCGCCACATACCTGTGCGCCGTGCGGCCTCTGTACGG
AGGCTCTTATATCCCAACCTTCGGCAGAGGCACAAGCCTGATCGTGCACC
CTTACATCCAGAACCCAGACCCCGCCGTGTATCAGCTGCGGGACAGCAAG
TCCTCTGATAAGTCCGTGTGCCTGTTCACCGACTTTGATTCCCAGACAAA
CGTGAGCCAGAGCAAGGACTCTGACGTGTACATCACCGACAAGTGCGTGC
TGGATATGAGAAGCATGGACITTAAGTCCAACTCTGCCGTGGCCTGGAGC
AATAAGTCCGATTTCGCCTGCGCCAACGCGTTTAACAATAGCATCATCCC
CGAGGATACATTCTTTCCTTCCCCAGAGAGCTCCTGTGACGTGAAGCTGG
TGGAGAAGAGCTTCGAGACAGATACAAACCTGAATTTTCAGAACCTGCTG
GTCATCGTGCTGCGGATCCTGCTGCTGAAGGTGGCCGGCTTCAATCTGCT
GATGACCCTGAGACTGTGGTCTAGCAGGGCAAAGCGCAGCGGATCCGGAG
CAACAAACTTTAGCCTGCTGAAGCAGGCAGGCGACGTGGAGGAGAATCCA
GGACCGCGGATGTCTATCGGCCTGCTGTGCTGTGCCGCCCTGAGCCTGCT
GTGGGCAGGACCAGTGAACGCAGGAGTGACCCAGACACCCAAGITCCAGG
TGCTGAAGACCGGCCAGTCTATGACACTGCAGTGCGCCCAGGACATGAAT
CACGAGTACATGAGCTGGTATCGGCAGGATCCTGGCATGGGCCTGAGACT
GATCCACTACTCCGTGGGAGCAGGAATCACCGACCAGGGAGAGGTGCCAA
ACGGCTATAACGTGAGCAGGAGCACCACAGAGGATTTCCCACTGAGGCTG
CTGTCTGCCGCACCTTCTCAGACAAGCGTGTACTTTTGCGCCTCCTCTTA
TGTGGGCAACACCGGCGAGCTGTTCTTTGGCGAGGGCTCCAGGCTGACAG
TGCTGGAGGACCTGAATAAGGTGTTCCCCCCTGAGGTGGCCGTGTTTGAG
CCCTCTGAGGCCGAGATCAGCCACACCCAGAAGGCCACCCTGGTGTGCCT
GGCAACCGGCTTCTTTCCTGATCACGTGGAGCTGTCCTGGTGGGTGAACG
GCAAGGAGGTGCACTCTGGCGTGTGCACAGACCCACAGCCCCTGAAGGAG
CAGCCAGCCCTGAATGACTCGAGATACTGCCTGAGCAGCCGCCTGAGGGT
CTCGGCCACCTTCTGGCAGAACCCCCGCAACCACTTCCGCTGTCAAGTCC
AGTTCTACGGGCTCTCGGAGAATGACGAGTGGACCCAGGATAGGGCCAAA
CCCGTCACCCAGATCGTCAGCGCCGAGGCCTGGGGTAGAGCAGACTGTGG
CTTTACCTCGGTGTCCTACCAGCAAGGGGTCCTGTCTGCCACCATCCTCT
ATGAGATCCTGCTAGGGAAGGCCACCCTGTATGCTGTGCTGGTCAGCGCC
CTTGTGTTGATGGCCATGGTCAAGAGAAAGGATTTCTGA.

The resulting engineered TCR encoded by the construct is referred to hereinafter as eTCR. The DNA sequence of the construct encoding the eTCR chains (SEQ ID NO: 25) was cloned into the MSGV-1 retroviral expression vector. MSGV-1 is a derivative of the murine stem cell virus (MSCV)-based splice-gag vector (pMSGV), which uses a MSCV long terminal repeat (LTR). Stable retroviral producer clones were generated by transfection into Eco-Phoenix cells and the vector supernatants were subsequently used to transduce PG13 packaging cells. Briefly, plasmid stock of eTCR was generated for transfection using DH5α E-Coli bacteria. Transfection to Eco-Phoenix cells was done by Jetprime reagent and 2 μg of purified eTCR construct plasmid. Virions from Eco-Phoenix cells were collected two days following transfection. Then, transduction of PG13 cells was performed using the spinoculation approach with retronectin reagent. Subsequently, virions collected from selected PG13 cells were spinoculated with retronectin and used to transduce healthy donor PBMCs.

The functionality of the eTCR was evaluated and determined to be highly effective against NY-ESO-1-expressing tumor cells, as described in Example 2 blow. Example 3 describes the selection of an eTCR subclone for the manufacture of clinical-grade cell compositions, and the generation of a master cell bank stably expressing the subclone. Examples 4-5 describe the development and characterization of the clinical-grade cell compositions and protocols for their preparation, and Examples 6-9 describe in vivo pre-clinical and clinical evaluation of these cell compositions.

### Example 2. Engineered TCR Exhibits Improved Functional Activity

To assess the functionality of the eTCR, its ability to induce antigen-specific stimulation and activity in transduced lymphocytes was evaluated and compared to that of control TCRs. To this end, primary peripheral blood mononuclear cells (PBMC)) of human donors were activated with an anti-CD3 antibody (OKT3) and IL-2, and were subsequently transduced with a retroviral vector encoding the tested TCR, as described in greater detail in Example 3 below. In addition to cells transduced with a vector encoding the eTCR, control cells were transduced with a retroviral vector encoding either a truncated version of nerve growth factor receptor (NGFR) as a negative control, or with a hitherto known native NY ESO-1 TCR directed to the same epitope as the eTCR, termed 1G4, as a positive control. The nucleic acid sequences encoding the variable regions of the α and β chains (VRα and VRs) of the 1G4 control TCR are as follows:

(1G4 VRα,)
SEQ ID NO: 26
ATGGAGACCCTCTTGGGCCTGCTTATCCTTTGGCTGCAGCTGCAATGGGT
GAGCAGCAAACAGGAGGTGACGCAGATTCCTGCAGCTCTGAGTGTCCCAG
AAGGAGAAAACTTGGTTCTCAACTGCAGTTTCACTGATAGCGCTATTTAC
AACCTCCAGTGGTTTAGGCAGGACCCTGGGAAAGGTCTCACATCTCTGTT
GCTTATTCAGTCAAGTCAGAGAGAGCAAACAAGTGGAAGACTTAATGCCT
CGCTGGATAAATCATCAGGACGTAGTACTTTATACATTGCAGCTTCTCAG
CCTGGTGACTCAGCCACCTACCTCTGTGCTGTGAGGCCCACATCAGGAGG
AAGCTACATACCTACATTTGGAAGAGGAACCAGCCTTATTGTTCATCCG;
and
(1G4 VRβ,)
SEQ ID NO: 27
ATGAGCATCGGCCTCCTGTGCTGTGCAGCCTTGTCTCTCCTGTGGGCAGG
TCCAGTGAATGCTGGTGTCACTCAGACCCCAAAATTCCAGGTCCTGAAGA
CAGGACAGAGCATGACACTGCAGTGTGCCCAGGATATGAACCATGAATAC
ATGTCCTGGTATCGACAAGACCCAGGCATGGGGCTGAGGCTGATTCATTA
CTCAGTTGGTGCTGGTATCACTGACCAAGGAGAAGTCCCCAATGGCTACA
ATGTCTCCAGATCAACCACAGAGGATTTCCCGCTCAGGCTGCTGTCGGCT
GCTCCCTCCCAGACATCTGTGTACTTCTGTGCCAGCAGTTACGTCGGGAA
CACCGGGGAGCTGTTTTTTGGAGAAGGCTCTAGGCTGACCGTACTG.

Next, the transduced cells were co-cultured with target tumor cells, either expressing or not expressing the NY-ESO-1 target antigen. The target cells included two types of melanoma tumor lines, namely A375 and 624.38, which are NY-ESO-1/HLA-A-0201 positive lines varying in their level of NY-ESO-1 expression. As a negative control, the 888A2 melanoma cell line, not expressing NY-ESO-1, was used. Incubation was performed at an effector:tumor cell ratio of 1:1 (100,000 cells from each population) for 24 hours. Following incubation, the levels of cytokines secreted to the media, as well as of activation markers on the transduced lymphocytes, were evaluated.

### Cytokine Secretion

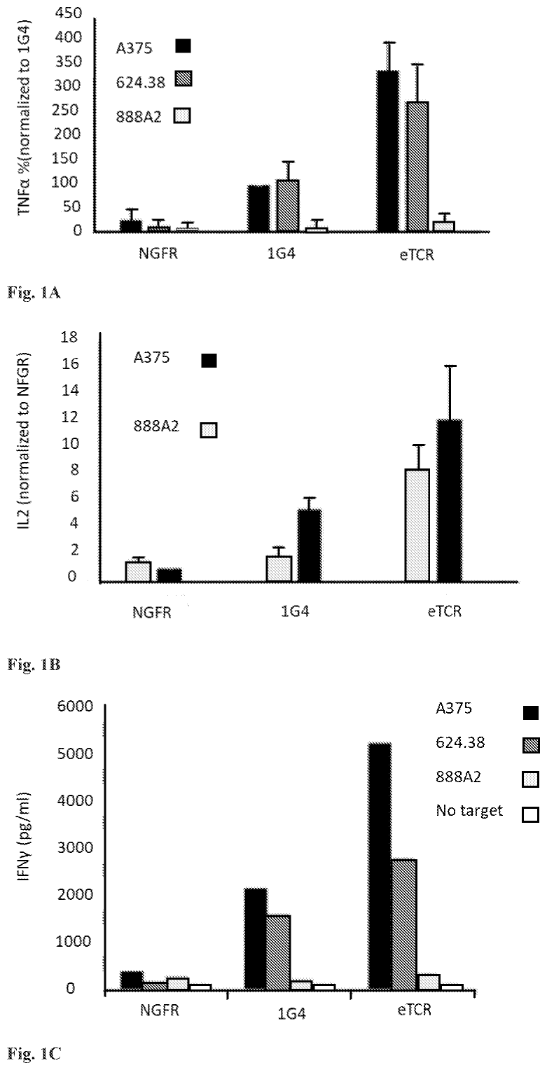

The secretion levels of tumor necrosis factor (TNFα), interleukin 2 (IL2) and interferon 7 (IFN7) to the co-culture supernatant were measured by ELISA. Results are presented in FIGS. 1A-1C respectively, in which the 1G4 TCR appears as 1G4. In FIG. 1A, TNFα concentrations are presented as mean t SEM, n=3, with three different donors; normalized to the activity of the 1G4 construct against A375 melanoma cells (with an average secretion of 567 pg/ml). In FIG. 1B, IL2 concentrations are presented as mean t SEM, n=2, with two different donors; normalized to the activity of negative control construct against A375 cells (with an average secretion of 119 pg/ml). In FIG. 1C, IFNγ concentrations are presented in pg/ml for the abovementioned co-cultures or with no target cells as a negative control.

As can be seen in FIGS. 1A-1C, transduction of either eTCR or 1G4 TCR resulted in enhanced cytokine secretion in the presence of NY-ESO-1 expressing cells as compared to the amounts secreted in the presence of unrelated tumor cells. In comparison, transduction with the negative control NGFR construct did not cause an enhancement in cytokine secretion, and these cells retained low basal levels in the presence of all types of target cells.

Furthermore, as can be seen in these figures, eTCR-transduced cells secreted remarkably higher levels of cytokines in the presence of their cognate target cells even as compared to cells transduced with the 1G4 TCR. For example, as can be seen in FIG. 1A, a statistically significant difference (p=0.0026) between the TNFα levels secreted by eTCR-transduced cells and by those transduced with the 1G4 TCR, calculated using a paired Student's t-test, was observes. Similarly, FIGS. 1B and 1C show marked enhancements in the magnitude of antigen-induced secretion of IL2 and IFNγ between the eTCR and 1G4-transduced groups.

### Activation Markers

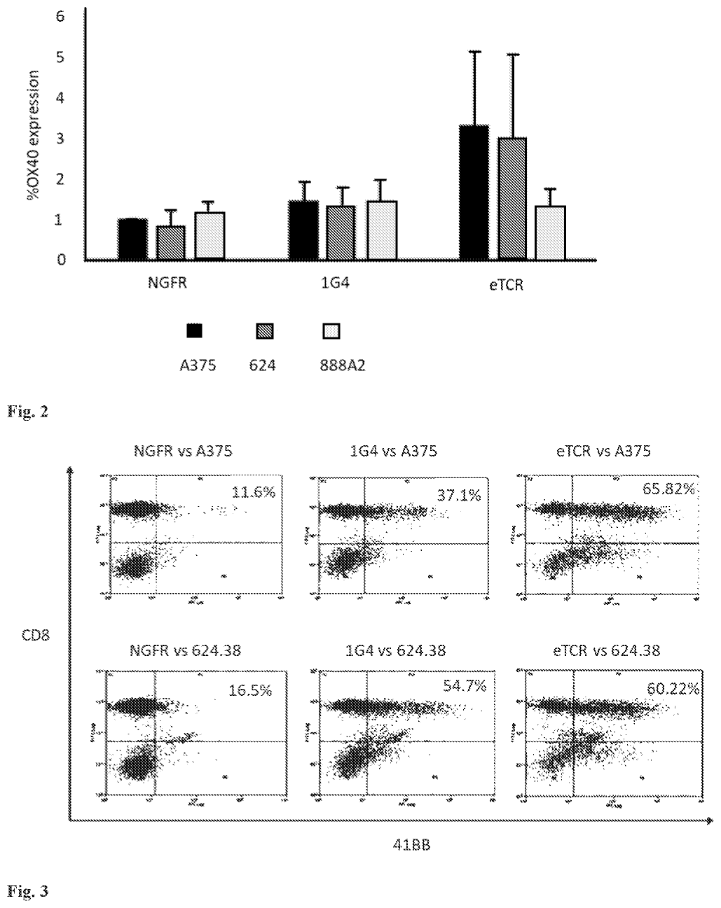

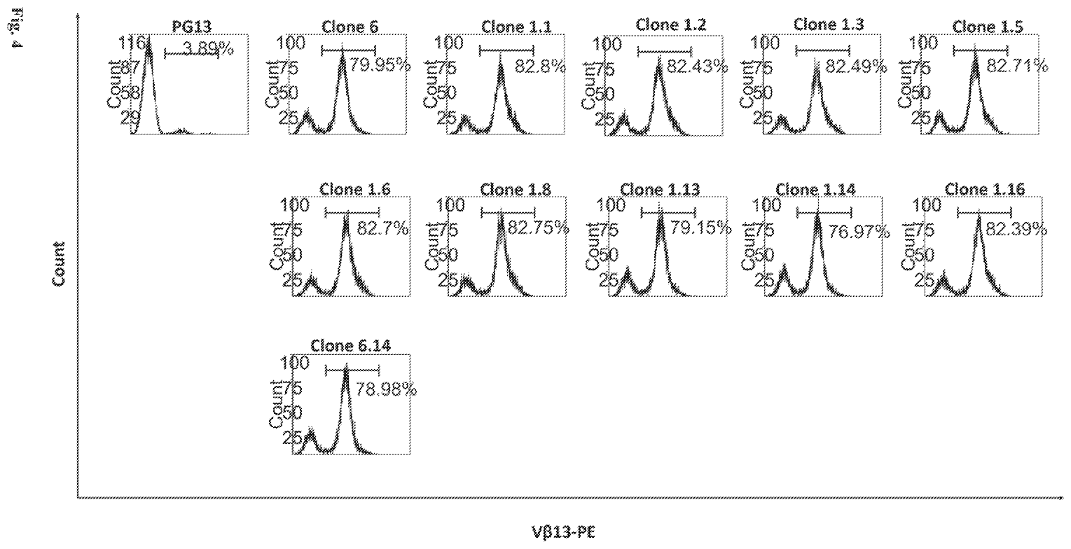

OX40 (CD134) and 4-1BB (CD137) are cell surface proteins expressed on activated T cells, thus they are considered T cell activation markers wherein their expression level serves as an indicator for T cell activation. Thus, OX40 and 4-1BB expression levels were determined following the co-culture of the eTCR, 1G4 TCR, or NGFR-transduced T cells with the target cells as described above. To this end, OX40 and 41BB were stained with suitable antibodies and measured by fluorescence activated cell sorting (FACS). Results for the OX40 are presented in FIG. 2 as mean+SEM, n=3, with three different donors; normalized to the activity of the NGFR construct against A375, wherein cell line 624.38 appears as 624. FIG. 3 presents FACS plots of 4-1BB expression in CD8⁺ cells transduced with either eTCR, 1G4 TCR, or NGFR expression vectors.

As can be seen in FIG. 2, it is clear that the eTCR transduced cells exhibited high OX40 expression levels, which were enhanced in the presence of NY-ESO-1-expressing tumor cells. Surprisingly, OX40 expression was remarkably higher in the eTCR transduced cells than in the 1G4 transduced cells, which exhibited only a slight elevation compared to the NGFR-transduced cells.

As can be seen in FIG. 3, a similar difference was also detected with respect to the 4-1BB expression levels; it is clear that the eTCR transduced cells exhibited higher 4-1BB expression levels compared to the 1G4 transduced cells co-cultured with either of the specific target cells lines, A375 and 624.38.

### CD8-Independent Activity

CD4⁺ cells were separated from PBMCs by magnetic beads. The purified population was transduced as described above and was found to express the eTCR at high levels. Functionally, these cells showed the ability to secrete IFN-γ (2174 pg/mL) in response to target melanoma cells A375 expressing the antigen. These results indicate that CD4⁺ T cells expressing the NY-ESO-1 TCR can be functionally reactive to melanoma tumor cells, as were the CD8⁺ cells (which secreted 2779 pg/mL IFN-γ under the same experimental settings).

### Functional Avidity

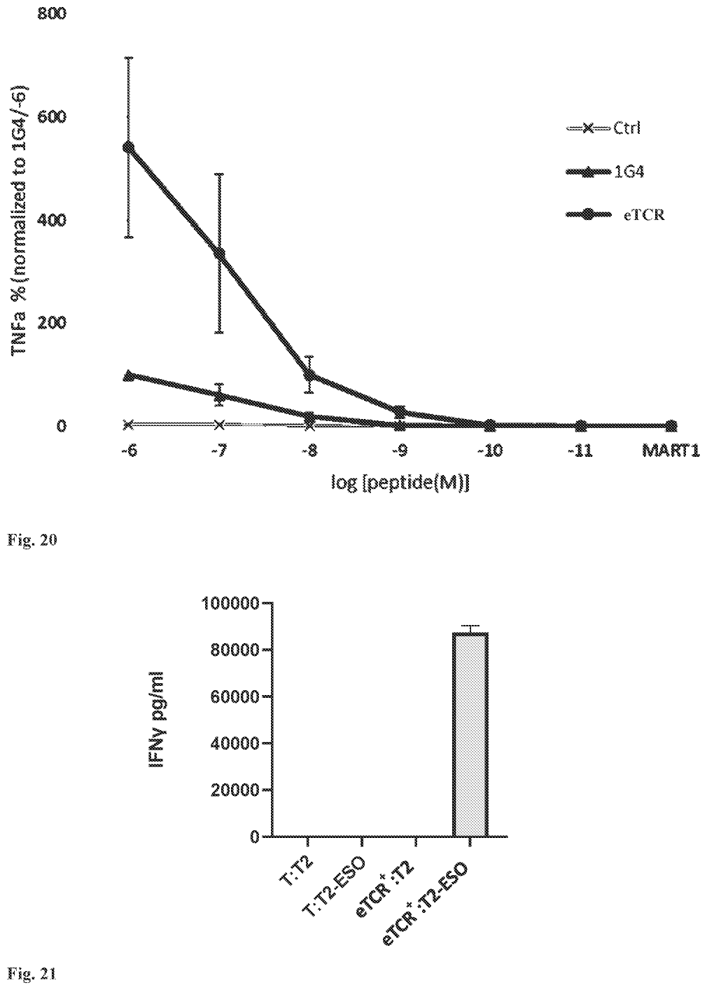

T-cells expressing either the 1G4 TCR or the eTCR were co-cultured with T2 cells pulsed with different concentrations of the NY-ESO-1 peptide (SEQ ID NO: 21) as indicated in FIG. 20, or with a MART-1 peptide as a negative control (1 μM). Following an overnight co-culture, the secretion of TNFα was measured in the culture supernatant. Results are presented in FIG. 20, in which TNFα levels (TNFa %) were plotted as a function of log[peptide concentration], and normalized to the levels measured in 1G4-expressing cells at the 1 μM concentration. The results are plotted as mean+SEM (n=4).

As cand be seen in FIG. 20, both eTCR-expressing cells and 1G4-expressing cells exhibited a dose-dependent enhancement in TNFα secretion in the presence of their cognate antigen. However, expression of the eTCR was associated with significantly higher target-induced TNFα secretion compared to cells expressing 1G4, in the presence of the T2 cells pulsed with NY-ESO-1 peptide concentrations ranging from about 1×10⁻⁶ M to about 1×10⁻¹⁰ M. Remarkably, enhancement of about 3-12-fold with an average of about 6-fold in TNFα secretion could be measured in the presence of all tested NY-ESO-1 peptide concentrations.

Thus, as demonstrated herein, the construct of the invention was found to be characterized by high functional avidity. Further, the construct exhibited marked enhancement in functional avidity as compared to a hitherto-known TCR directed to the same antigen, exhibiting an average improvement of about 6-fold at physiologically-relevant antigen levels.

In summary, transduction of the eTCR into lymphocytes derived from human PBMC resulted in a significantly enhanced tumor-specific response, not only as compared to an irrelevant TCR (NGFR), but also as compared to a NY-ESO-1 TCR directed to the same epitope as the eTCR (1G4). Transduction with the novel engineered construct resulted in enhanced secretion of cytokines that are major participants in anti-tumor immunity, in improved functional avidity, and in enhanced expression of activation markers, indicative of improved functionality of the eTCR. The results also demonstrate that the eTCR is remarkably capable of exerting tumor-specific reactivity in a CD8-independent manner.

### Example 3. Subcloning and Production of Viral Vector for Clinical Manufacturing

A stock of the eTCR expression vector was generated for transfection using DH5α E-Coli bacteria (ThermoFischer, Cat #18265-017) and transfected to Phoenix-ECO cells, essentially as described in Example 1. The ability of the vector to transduce PG13 cell was demonstrated, and 12 single-cell clones were produced. The expression and function potential of the produced clones and subsequent subclones were examined as detailed below.

For the selection of high-eTCR expressing clones, the transduction efficacy was evaluated by measuring the expression of the β chain of the eTCR on PBMC transduced with supernatants derived from each of the tested clones. To this end, cryopreserved PBMC from healthy donors were thawed, suspended in culture medium (433 ml RPMI medium, 50 ml Human Serum, 5 ml GlutaMAX-ICTS (100×), 0.5 ml β-mercaptoethanol, 12 ml Hepes and 300 IU/mL IL-2) and washed by centrifugation. Cells were then re-suspended in culture medium at a concentration of 2×10⁶ cells/mL, and activated for two days in the presence of an anti-CD3 antibody (OKT3, 50 ng/ml) and IL2. Transduction of the activated cells was performed in 24-well plates pre-coated with Retronectin and blocked with 2% BSA in PBS. Next, 2 mL of PG13-derived viral supernatants were added to the coated wells, centrifuged at 2000G for 2 hours, and supernatants were collected and treated by sodium hypochlorate (to inactivate the left-over retroviral particles). Cells resulting from the PBMC activation were added (500,000 cells per well) and the plates were centrifuged and incubated at 37° C. The following day, cells were washed, resuspended in culture medium plated in 6-well plates and incubated at 37° C. for 72 hours. The resulting cells were analyzed for eTCR expression at day 4 following transduction.

FIG. 4 shows exemplary results of a flow cytometry assay measuring the level of expression of the eTCR corresponding to the initial 12 clones, using a PE-conjugated anti-Vβ13 antibody directed to the specific beta chain of the eTCR. In FIG. 4, PG13 cells transduced only with medium were used as negative control, and the percentile of Vβ13-PE staining is indicated in each plot.

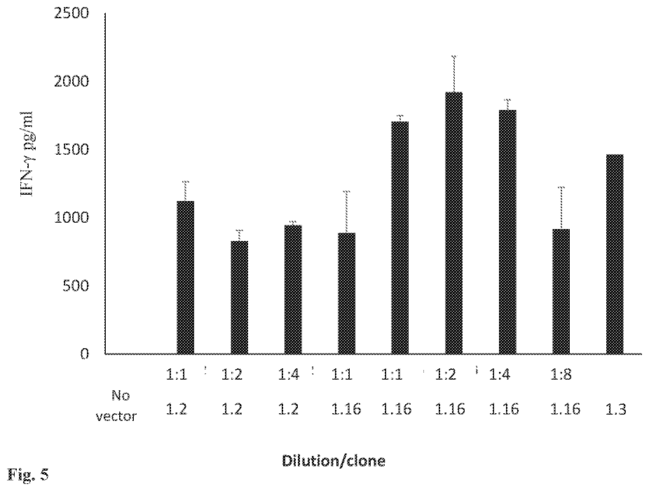

Selected clones were further subcloned in PG13 cells and these subclones (all determined to show adequately high expression levels as their originating clones) were used to transduce lymphocytes in order to examine their functional efficacy. For the IFN-γ secretion assay, the vector was also tittered, and lymphocytes were transduced with vector from the selected clones diluted at 1:1, 1:2, 1:4 or 1:8. Next, the transduced lymphocytes prepared from thawed healthy donor PBMC as detailed above were co-cultured with melanoma A375 cells expressing NY-ESO-1 at a 1:1 effector to target (E:T) ratio for 24 hours. Following culture, supernatants were examined for IFN-γ secretion by ELISA. In addition, killing of melanoma A375 cells by transduced lymphocytes (with the undiluted vector) was assayed by culturing transduced lymphocytes with A375 cells at a 1:1 E:T ratio for 1.5 hours. Melanoma cells were stained with the cell tracer marker DDAO, and for caspase-3 as an indicator of apoptosis. Cells were examined for % caspase-3 and DDAO by FACS. Representative results of the IFN-γ secretion assay are shown in FIG. 5, in which clones 1.2, and 1.16 appear in the different dilutions (1:1, 1:2, 1:4 or 1:8), undiluted clone 1.3 was used as a positive control, and PG13 cells transduced only with medium and not with a vector, referred herein after as no vector (FIG. 5) or PG13 (FIG. 6), were used as a negative control. Representative results of the caspase-3 measurements are shown in FIG. 6, in which “Poly” refers to the original polyclonal eTCR supernatant from the PEG13 initial transduction before sub-cloning.

The results teach that the novel construct of the invention facilitated the expression of the eTCR in an efficient manner, which enabled the transduced cells to exert a specific anti-cancer activity. In particular, clone 1.16 was determined to provide high expression levels, high IFN-γ secretion levels and high levels of apoptosis induction in melanoma A375 cells as measured by caspase-3 levels, was thus chosen for further manufacturing of clinical-grade viral master cell bank (MCB).

The selected PG13 clone was expanded to generate the MCB (each stock comprising an average of at least 300×10⁶ cells), which was tested for absence of mycoplasma and for sterility, and the retroviral supernatant was collected and used in the following examples.

### Example 4. Transduction Conditions for Manufacturing the Clinical-Grade Cell Composition

Clone 1.16 produced from the MCB served as the eTCR vector source for the following experiments, as part of the development and production of a GMP clinical-grade composition. In order to determine suitable conditions for transduction, the following experiments were conducted.

Several batches of eTCR supernatants, harvested at different times (12 or 24 hours from PG13 cells grown in different temperatures (32 or 37° C.) were subjected to different dilutions (1:2, 1:5 or 1:10). PBMC obtained from apheresis were activated in the presence of an anti-CD3 antibody and IL-2 essentially as described in Example 3, and two days later transduced with the vector collected in the different batches and in the different dilutions, using the spinoculation method with Retronectin essentially as described in Example 3. eTCR expression and function were assessed at days 6, 13, 20 post transduction, as follows.

Transduction efficacy of the different dilutions of eTCR vector harvests in different transduction conditions was evaluated using the Vβ13-PE antibody and analyzed by FACS, essentially as described in Example 3. Table 2 shows exemplary results of the post-transduction eTCR expression levels, presented as precent of Vβ13 expression.

TABLE 2
post-transduction eTCR expression levels as
function of different transduction conditions
% Vβ13 in lymphocytes

| | | | Day 6 | | Day 13 | | Day 20 | |
|---|---|---|---|---|---|---|---|---|
| Temp. (° C.) | Time | Harvest | 1:02 | 1:05 | 1:02 | 1:05 | 1:02 | 1:05 |
| 37 | 12 | 1 | 75.16 | 58.48 | 73.15 | 60.02 | 75.51 | 58.87 |
| 37 | 12 | 2 | 75.02 | 59.85 | 74.31 | 63.02 | 77.28 | 62.56 |
| 37 | 12 | 3 | 79.59 | 66.56 | 77.55 | 65.65 | 81.54 | 68.63 |
| 37 | 12 | 4 | 83.11 | 75.22 | 77.13 | 71.75 | 85.24 | 79.57 |
| 37 | 24 | 1 | 70.75 | 61.46 | 72.35 | 66.01 | 75.7 | 66.16 |
| 37 | 24 | 2 | 79 | 72.76 | 77.46 | 75.1 | 83.38 | 77.34 |
| 37 | 24 | 3 | 80.89 | 79.17 | 77.83 | 76.02 | 84.12 | 81.11 |
| 37 | 24 | 4 | 81 | 77.3 | 77.69 | 74.76 | 82.64 | 79.73 |
| 32 | 12 | 1 | 65.2 | 44.58 | 66.75 | 51.4 | 67.76 | 44.11 |
| 32 | 12 | 2 | 74.17 | 57.41 | 77.57 | 60.91 | 77.2 | 60.9 |
| 32 | 12 | 3 | 74.57 | 61.52 | 74.4 | 63.54 | 78.55 | 62.97 |
| 32 | 12 | 4 | 75.53 | 59.75 | 73.78 | 64.52 | 79.39 | 67.03 |
| 32 | 24 | 1 | 78.94 | 61.91 | 78.91 | 66.82 | 81.84 | 67.41 |
| 32 | 24 | 2 | 80.32 | 72.96 | 78.89 | 76.51 | 83.64 | 78.7 |
| 32 | 24 | 3 | NA | 72.44 | 80.03 | 75.45 | 84.09 | 78.59 |
| 32 | 24 | 4 | 78.92 | 76.29 | 79.93 | 77.62 | 82.27 | 81.12 |
| No dilution | NA | NA | 73.01 | ND | 73.42 | ND | 74.56 | ND |
| No vector | NA | NA | 3.76 | ND | 21.14 | ND | 4.32 | ND |
| HVB1 | NA | NA | 60.25 | ND | 61.38 | ND | 61.65 | ND |

In Table 2, “No dilution” indicates PG13 supernatants obtained from the MCB, without dilution. “No vector” indicates PG13 supernatants obtained from cells not carrying a TCR expression vector. “HVB1” represent a non-diluted stock of 1.16 vector derived from the MCB that was used in Example 5. ND—not determined, NA—not available.

The results show an unexpectedly high transduction efficacy of the MCB derived eTCR at dilutions of 1:2 and 1:5, in all conditions. The highest transduction efficiencies were detected with the eTCR vector harvested at 24-hour intervals.

Functionality and activity assessments were carried out by measuring IFN-γ secretion and caspase-3 levels, essentially as described in Example 3. Briefly, the transduced cells were co-cultured for 24 hours with A375 cells (expressing NY-ESO-1) at a 1:1 E:T ratio. Following the co-culture, supernatants from the cells were collected at days 6, 13 and 20 post transduction and IFN-γ secretion levels were measured by ELISA. For evaluation of the tumor killing capacity, transduced PBMC were co-cultured for 1.5 hours with A375 cells expressing NY-ESO-1 at 1:1 E:T ratio. Following the co-culture at days 6, 13 and 20 post transduction, caspase-3 was stained in A375 melanoma cells for FACS detection as an indicator of an apoptosis.

The results demonstrated that IFN-γ secretion levels were significantly higher compared to the negative control, indicating specific target-induced activity. The secretion levels were only little affected by the different conditions, with modest elevation in the group transduced at 32° c. with a 24h interval harvest. Similar results were obtained with the caspase-3 staining assay. The levels were higher compared to the negative control, indicating specific target-induced activity, and a drop in caspase-3 levels was detected on day 20 post transduction.

Copy number (CPN) of the eTCR vector in cells transduced with supernatants from sixteen different harvests, differing in harvest time and culture temperature, was further determined. PBMC from two healthy donors were transduced with eTCR vector and co-cultured with melanoma A375 cells. Copy number was detected using RT-PCR assay. The Results are presented in Table 3 as CPN/Transduced Cell, n=2.

TABLE 3
Average copy number (CPN)

Dilution

| Harvest | 1:02 | 1:05 | 1:10 |
|---|---|---|---|
| 1 | 5.51 ± 0.93 | 5.79 ± 0.55 | 1.80 ± 2.55 |
| 2 | 4.89 ± 1.56 | 2.39 ± 0.04 | 2.23 ± 1.11 |
| 3 | 2.89 ± 0.15 | 2.65 ± 0.59 | 2.28 ± 0.81 |
| 4 | 3.75 ± 0.87 | 2.42 ± 0.37 | 1.66 ± 1.075 |
| 5 | 2.66 ± 0.59 | 2.69 ± 0.67 | 2.40 ± 0.05 |
| 6 | 3.12 ± 0.71 | 2.75 ± 0.06 | 2.92 ± 0.79 |

As can be seen in Table 3, most test conditions generated CPN values of about 5 CPN or less. As CPN≤5 is a regulatory standard in clinical trials it is clear that the eTCR construct derived from the MCB provides for high surface expression with adequate functionality and activity as described above while complying with regulatory standards.

Multiplicity of infection (MOI) was calculated using the following formula:

$$P ( k ) = {e}^{- m} {m}^{k} / k !$$

where “P(k)” is the fraction of cells infected by k virus particles, and “m” is the MOI.

MOI was measured at day 6 post transduction, n=2. The average results in the different batches ranged from 1.14±0.02 to 1.47±0.06 in the 1:2 dilution, from 1.21±0.06 to 1.03±0.12 in the 1:5 dilution and from 0.74±0.02 to 0.94±0.07 in the 1:10 dilution.

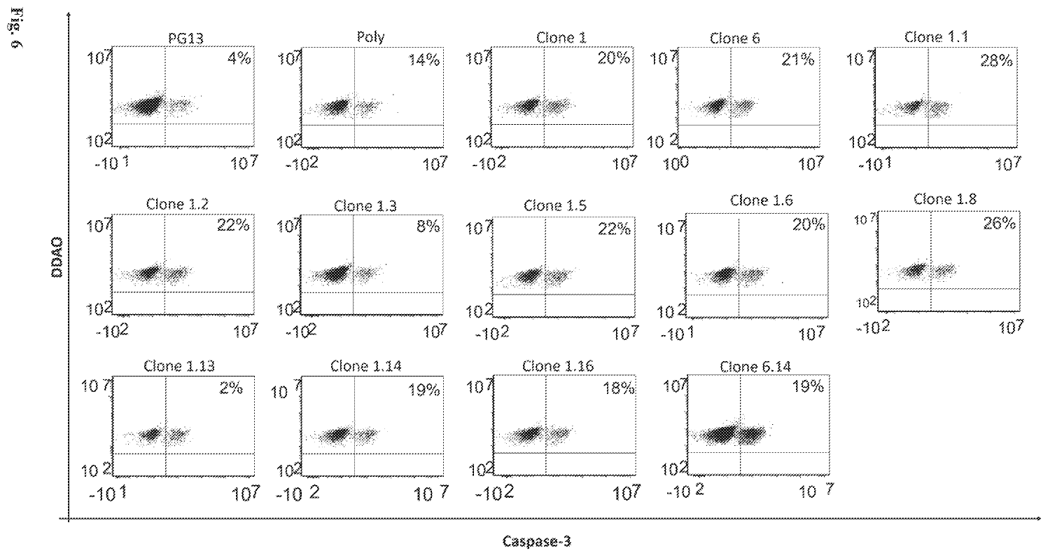

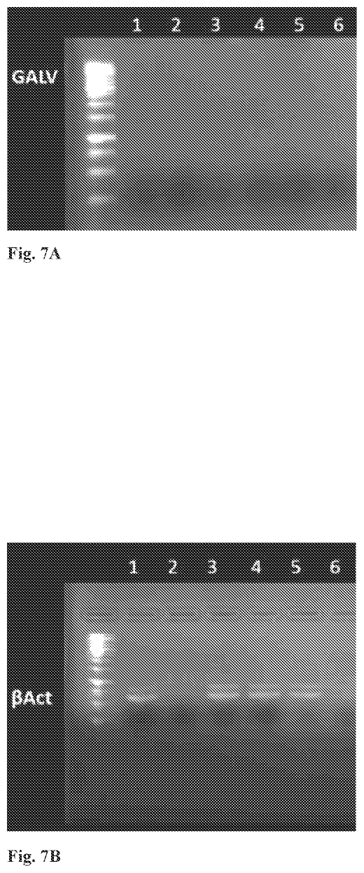

Finally, the presence of replication competent retrovirus (RCR) was examined in 6 random samples on day 20 post transduction, by PCR using primers for GALV (the viral envelope protein) as an indicator of RCR, and for beta-actin (βAct) as a positive control. PCR products were run on an 1% agarose gel. Results are presented in FIG. 7A-7B. FIG. 7A, PCR using primers for GALV (the viral envelope protein) as an indicator of RCR. FIG. 7B, PCR using primers and for beta-actin (βAct) as a positive control. It is clear from the results that none of the samples expressed GALV, indicating no RCR being present in any of the tested samples.

Based on the results of the experiments discussed herein, the following transduction conditions were selected for the experiments described in the following Examples: transduction temperature of 32° C., virion harvest time of 24 hours, vector dilution of 1:5 and MOI=1.

### Example 5. Expansion Conditions for Manufacturing Clinical-Grade Cell Compositions

Next, various protocols for manufacturing the clinical-grade adoptive cell transfer (ACT) composition were examined. To this end, fresh or thawed PBMC samples obtained by apheresis from healthy donors were subjected to various activation and expansion protocols, as described in further detail below and summarized in Table 4. Transduction with the eTCR vector was performed following the activation step and prior to the expansion step, under the conditions as described in Example 4.

The first protocol involved activation with an anti-CD3 antibody (OTK3) and IL-2 at the concentrations indicated in Table 4 for two days (in complete medium containing RPMI, 10% human serum, glutamine, Hepes and b-mercaptoethanol) and expansion in the presence of enhanced IL-2 levels and irradiated allogeneic PBMC, serving as feeder cells. In this protocol, no antibodies were supplemented in the expansion stage, and the only cell stimulating agents supplemented were IL-2 and the feeder cells. The cell media was changed gradually during the expansion step from complete medium to AIMV (Gibco) with 5% Human serum. This protocol is referred to herein as “Anti-CD3+feeders”. Lymphocytes transduced with a vector-free supernatant (“no vector”) served as a negative control.

The second and third protocols involved activation and expansion using surface-bound anti-CD3 and anti-CD28 antibodies and IL-2. In particular, the second protocol utilized the commercially available product T Cell TransAct™ (Milteny) comprising anti-CD3 and anti-CD28 antibodies conjugated to nanoparticles (referred herein throughout as “Transact”), and the third protocol utilized DynaBeads (Thermo Scientific), employing superparamagnetic bead-conjugated anti-CD3 and anti-CD28 antibodies. In these protocols, the surface-bound antibodies were introduced in the activation stage only, and the expansion step included only supplementation of IL-2 and gradual replacement of the culture medium as described in the first protocol. These protocols are also referred to as the “Transact” and “DynaBeads” protocols, respectively.

TABLE 4
Activation and expansion protocols

| Group | Transduction | Activation | Expansion |
|---|---|---|---|
| Anti-CD3 + feeders | eTCR 1:2 dilution | Anti-CD3 (50 ng/mL) + | Feeders + IL-2 3,000 |
| | | IL-2 (300 IU/mL) | IU/mL |
| Transact + | eTCR 1:2 dilution | Transact + IL-2 (3,000 | IL-2 3,000 IU/mL |
| IL-2 3,000 IU/mL | | IU/mL) | |
| Dynabeads + | eTCR 1:2 dilution | Dynabeads + IL-2 (200 | IL-2 200 IU/mL |
| IL-2 200 IU/mL | | IU/mL) | |
| Anti-CD3 + feeders | No vector | Anti-CD3 (50 ng/mL) + | Feeders + IL-2 3,000 |
| | | IL-2 (300 IU/mL) | IU/mL |
| Transact + | No vector | Transact + IL-2 (3,000 | IL-2 3,000 IU/mL |
| IL-2 3,000 IU/mL | | IU/mL) | |
| Dynabeads + | No vector | Dynabeads + IL-2 (200 | IL-2 200 IU/mL |
| IL-2 200 IU/mL | | IU/mL) | |
| Feeders only | ND (not done) | ND | IL-2 3,000 IU/mL |

Surface expression of the eTCR following the different activation and expansion protocols was evaluated by measuring Vβ13, using flow cytometry, at days 6, 13 and 20 post transduction, essentially as described in Example 3. Exemplary results of two independent experiments (experiment 1 and experiment 2) are presented in Table 5 below.

TABLE 5
eTCR expression following different expansion protocols
% Vβ13 in lymphocytes

| | Experiment 1 | | | Experiment 2 | | |
|---|---|---|---|---|---|---|
| Day post transduction | 6 | 13 | 20 | 6 | 13 | 20 |
| Anti-CD3 + feeders eTCR | 63.83 | 55.55 | 44.03 | 74.28 | 60.74 | 44.74 |
| 1:2 dilution | | | | | | |
| Transact IL-2 3,000 IU/mL | 80.97 | 85.1 | 81.4 | 79.17 | 83.48 | 70.19 |
| eTCR 1:2 dilution | | | | | | |
| Dynabeads IL-2 200 IU/mL | 84.18 | 87.04 | 90.41 | 81.9 | 82.59 | 75.17 |
| eTCR 1:2 dilution | | | | | | |
| Anti-CD3 + feeders, no | 3.55 | 5.38 | 4.73 | 3.82 | 4.93 | 7.03 |
| vector | | | | | | |
| Transact IL-2 3,000 IU/mL, | 3.06 | 2.61 | 2.96 | 3.53 | 2.99 | 5.02 |
| no vector | | | | | | |
| Dynabeads IL-2 200 IU/mL, | 3.91 | 3.94 | 4.42 | 4.21 | 4.75 | 6.04 |
| no vector | | | | | | |

As can be seen in Table 5, all protocols resulted in remarkably high transduction levels with surprisingly persistent retention of surface expression. Expression levels of the eTCR were high throughout the entire experiment and up until day 20 post transduction and across all protocols, with the Dynabeads protocol providing the highest levels and only modest decreases encountered by the end of the other protocols.

Next, the functional capacity of the cells produced by the different protocols was determined by evaluating antigen-induced IFN-γ secretion. To this end, the resulting cells were co-cultured with T2 cells loaded with 1 μg NY-ESO-1 peptide at E:T ratio of 1:1 for 24 hours. Following co-culture, supeUatants from the cells were collected at days 6, 13 and 20 post transduction and examined for IFN-γ secretion levels by ELISA. Exemplary results are presented in Table 6.

TABLE 6
Target-specific IFN-γ secretion following the different activation and
expansion protocols. The IFN-γ concentration are presented in fg/transduced cell.

| | | | Dynabeads | Transact | Anti- | Dyna | Transact | |
|---|---|---|---|---|---|---|---|---|
| | | | IL-2 200 | IL-2 3,000 | CD3⁺ | beads IL-2 | IL-2 3,000 | Anti-CD3⁺ |
| | | | IU/mL | IU/mL | feeders | 200 IU/mL | IU/mL | feeders |
| Exp. | Day | | eTCR 1:2 | eTCR 1:2 | eTCR 1:2 | No vector | No vector | No vector |
| 1 | 6 | Avg | 6.87 | 179.74 | 80.47 | 9.8 | 0 | 0 |
| | | SD | 0.45 | 37.85 | 1.13 | 12.81 | 23.97 | 0 |
| | 13 | Avg | 4.31 | 15.99 | 10.05 | 0 | 0 | 0 |
| | | SD | 1.04 | 1.89 | 1.53 | 0 | 0 | 0 |
| | 20 | Avg | 1.64 | 13.67 | 8.68 | 0 | 0 | 0 |
| | | SD | 0.81 | 1.25 | 3.90 | 0 | 0 | 0 |
| 2 | 6 | Avg | 3.10 | 34.86 | 23.32 | 0 | 0 | 0 |
| | | SD | 0.54 | 1.63 | 2.18 | 0 | 0 | 0 |
| | 13 | Avg | 0.38 | 4.76 | 4.61 | 2.17 | 1.49 | 0.90 |
| | | SD | 0.72 | 2.29 | 2.29 | 3.77 | 2.58 | 1.56 |
| | 20 | Avg | 0 | 0.86 | 0.26 | 0 | 0 | 0 |
| | | SD | 0 | 0.22 | 0.22 | 0 | 0 | 0 |

In Table 6, “Exp” indicates the experiment number (corresponding to the experiments described in Table 5 above), “Avg” indicates average, “SD” indicates standard deviation, and the production protocols are as detailed in Table 4 above.

As can be seen in Table 6, target-induced IFN-γ secretion was detected in the various eTCR-transduced groups, and in particular in the cell compositions produced by the Transact protocol and anti-CD3+feeders protocol.

For further examination of the effect of the activation and expansion protocols on the functionality of the GMP eTCR cell compositions, the cytotoxic activity of the cells was evaluated by the detection of apoptosis in cognate target cells. To this end, cells produced as described above were co-cultured with T2 cells loaded with 1 μg NY-ESO-1 peptide at E:T ratio of 1:1 for 1.5 hours. Subsequently, caspase-3 was stained in the co cultured T2 cells and detected by FACS at day 6, 13 and 20 post-transduction. Exemplary results are presented in Table 7.

TABLE 7
Caspase-3 expression (%) in NY-ESO-1-loaded target cells

| | | | Dynabeads | Transact | Anti- | Dynabeads | Transact | Anti- |
|---|---|---|---|---|---|---|---|---|
| | | | IL-2 200 | IL-2 3000 | CD3⁺ | IL-2 200 | IL-2 3000 | CD3⁺ |
| | | | IU/mL | IU/mL | feeders | IU/mL | IU/mL | feeders |
| Exp | Day | | eTCR 1:2 | eTCR 1:2 | eTCR 1:2 | No vector | No vector | No vector |
| 1 | 6 | Avg | 39.05 | 14.16 | 13.15 | 1.82 | 1.8 | 2.22 |
| | | SD | 1.37 | 0.2 | 0.56 | 0.26 | 0.23 | 0.18 |
| | 13 | Avg | 51.317 | 17.73 | 33.23 | 4.26 | 4.27 | 5.05 |
| | | SD | 1.52 | 1.39 | 3.8 | 0.34 | 0.33 | 0.66 |
| | 20 | Avg | 43.73 | 26.97 | 12.49 | 4.73 | 4.78 | 4.67 |
| | | SD | 3.96 | 6.22 | 2.62 | 0.79 | 1.76 | 1.72 |
| 2 | 6 | Avg | 63.01 | 30.07 | 38.65 | 4.66 | 5.69 | 6.05 |
| | | SD | 2.45 | 2.4 | 6.51 | 0.18 | 0.04 | 0.7 |
| | 13 | Avg | 36.1 | 6.79 | 47.59 | 2.41 | 2.67 | 5.87 |
| | | SD | 19.92 | 0.33 | 16.24 | 0.75 | 0.78 | 3.08 |
| | 20 | Avg | 59.05 | 40.06 | 43.33 | 12.64 | 10.10 | 12.17 |
| | | SD | 3.13 | 16.29 | 2.242 | 3.75 | 2.65 | 0.36 |

As can be seen in Table 7, antigen-specific target cell apoptosis was demonstrated in eTCR transduced cells manufactured according to each of the tested protocols, with particularly potent activity observed in the Dynabeads group.

In summary, results presented herein show that all manufacture protocols support the production of cell compositions characterized by high and persistent eTCR expression and potent anti-tumor activity.

Next, the compositions were further characterized with respect to the presence of specific subsets and phenotypes. For the characterization of different T cell subsets in the cell compositions manufactured by the above protocols, CD3⁺ cells obtained from each cell compositions were stained with different antibodies directed to CD45RA and CCR7 and analyzed by flow cytometry. A mixture of the following flourophore-conjugated antibodies was used for the analysis: CCR7-rPE, CD45RA-ECD, CD28-PC5.5, CD3-PC7, CD8-APC, CD62L-AF700, CD4-APC-Cy7, and CD27-BV421.

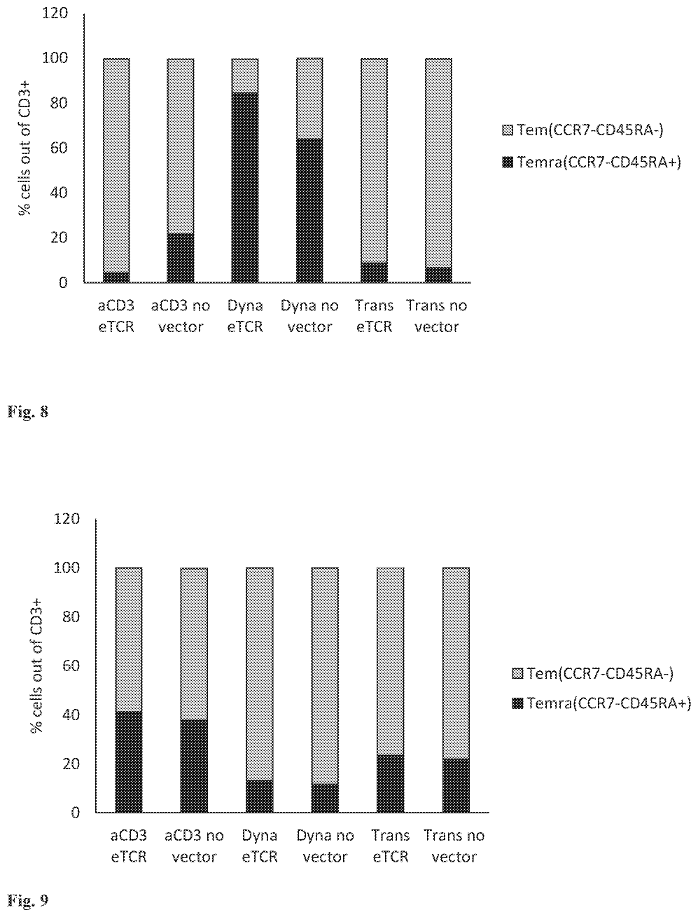

The staining provides for the characterization of four groups of T cells, as follows: naive T cells (Tn) expressing both CCR7 and CD45RA, and three groups of primed (memory) cells, namely central memory cells (T_{CM}) expressing CCR7 but not CD45RA, effector memory (T_{EM} gray bars) cells lacking expression of both CCR7 and CD45RA, and T_{EMRA} cells that express CD45RA but lack expression of CCR7 (black bars). Exemplary results, presented in FIGS. 8 and 9, correspond to experiments 1 and 2, respectively, at day 20 post transduction. Results are presented as the percentile of each group (T_{EM} or T_{EMRA}) out of the total number of CD3⁺ cells.

As can be seen in FIGS. 8-9, the resulting T cells adopted a differentiated effector phenotype, with negligible amounts of naïve cells detected at day 20. As can be further seen, T cells produced by the anti-CD3+feeders and Transact protocols adopted mainly a T_{EM} (CD45RA⁻CCR7⁻) phenotype with a smaller population of T_{EMRA} (CD45RA⁺CCR7⁻) cells, whereas T cells produced by the Dynabeads protocol exhibited a different phenotype with an increased proportion of T_{EMRA} (CD45RA⁺CCR7⁻) cells.

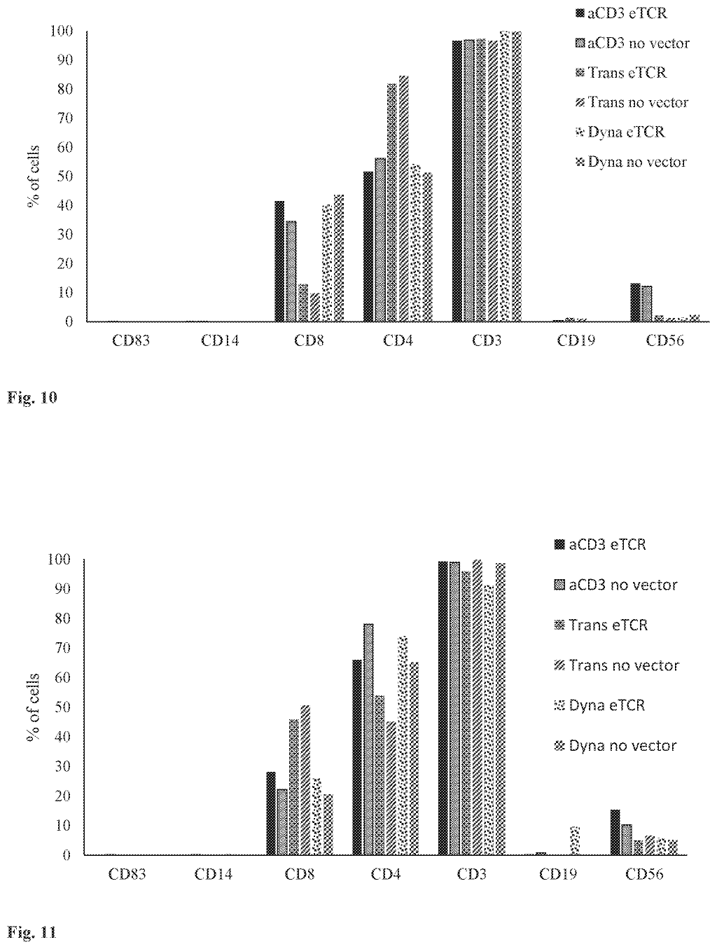

Further, the proportion of different subsets of immune cells in the various cell compositions was analyzed at days 13 and 20 post transduction. Cells were stained for CD3, CD4, CD8, CD14, CD19, CD83 and CD56 and analyzed by flow cytometry. Exemplary results corresponding to days 13 and 20 of experiment 2 are presented in FIG. 10-11, respectively (similar results were obtained in experiment 1).

As can be seen in these figures, the resulting cell compositions comprised of mainly CD3⁺ cells with minimal CD14⁺, CD83⁺ or CD19⁺ impurities (representing other cells such as macrophages and B cells). As can also be seen, all eTCR transduced groups surprisingly included considerable proportions of both CD4⁺ cells and CD8⁺ cells. It can also be seen that the relative proportion of CD8⁺ cells out of the entire T cell population (CD3⁺) was significantly lower in the Transact group at day 13 and 20 compared to the other groups.

Finally, copy number analysis of the eTCR vector in the cell compositions resulting from the different protocols of experiment 1 was assessed by qPCR. Copy number was 1.82 for the anti-CD3+feeders protocol, 0.93 for the Transact protocol and 1.89 for the Dynabeads protocol.

In summary, described herein is the production of a clinical-grade ACT composition expressing a newly-designed NY-ESO-1-specific TCR. The composition was characterized as exhibiting improved properties, including but not limited to: exceptionally high and prolonged retention of transgene expression at low vector copy numbers, expression on both CD4⁺ and CD8⁺ cells, high proportion of effector cells exhibiting desirable phenotypes, and potent antigen-specific activity.

Based on these results and considering all of the above factors, the CD3+feeders protocol was chosen for the manufacture of the clinical-grade ACT composition to be examined in the in vivo experiments described in Examples 6-8 below (compositions 1-3, respectively). Example 8 further describes the in vivo evaluation of ACT compositions produced using the other manufacture methods as detailed above.

A summary of the characteristics of ACT compositions produced for in vivo testing is presented in Table 8 below.

TABLE 8
Characteristics of ACT compositions

| ACT | Specification/ | | | |
|---|---|---|---|---|
| characteristics | Method | Composition 1 | Composition 2 | Composition 3 |
| Potency | IFNγ release | Week 2/Week 3 | Week 2/Week 3 | Week 2/Week 3 |
| | (ELISA, fg/TDC) | 25.26/8.96 | 10.05/8.68 | 4.61/1.17 |
| | Apoptosis | Week 2/Week 3 | Week 2/Week 3 | Week 2/Week 3 |
| | Cleaved Caspase3 | 57.27/44.63 | 33.23/12.49 | 47.59/43.33 |
| | killing assay (% of | | | |
| | target cells) | | | |
| Identity (%) | | | Week 2: 92.67 | Week 2: 96.62 |
| CD3⁺ CD19⁺ | | | CD3⁺/0.22 | CD3⁺/0.22 |
| CD56⁺ CD14⁺ | | | CD19⁺/15.32 | CD19⁺/13.2 |
| CD83⁺ | | | CD56⁺/4.24 | CD56⁺/0.26 |
| | | | CD14⁺/0 | CD14⁺/0 |
| | | | CD83⁺ | CD83⁺ |
| | | | Week 3: 98.24 | Week 3: 99.24 |
| | | | CD3⁺/0.05 | CD3⁺/0.17 |
| | | | CD19⁺/1.03 | CD19⁺/15.27 |
| | | | CD56⁺/0.01 | CD56⁺/0 |
| | | | CD14⁺/0 | CD14⁺/0.02 |
| | | | CD83⁺ | CD83⁺ |
| Purity (CD3⁺ T | | | Week 2: 92.67 | Week 2: 96.62 |
| cells, %) | | | Week 3: 98.24 | Week 3: 99.24 |
| Cell viability | Flow cytometry | | Week 2: 59.88 | Week 2: 82.36 |
| (%) | (SytoxBlue- cells) | | Week 3: 89.93 | Week 3: 94.08 |
| Transduction | Flow Cytometry | Week 2: 56.21 | Week 2: 55.55 | Week 2: 55.55 |
| (%) | Vβ13-PE | Week 3: 67.71 | Week 3: 44.03 | Week 3: 44.03 |
| CD8/CD4 (%) | | | Week 2: | Week 2: |
| | | | 35.55/59.96 | 51.58/41.54 |
| | | | Week 3: | Week 3: |
| | | | 4.7/92.6 | 65.92/28.09 |
| Injection | | Saline + 2% | Saline + 2% | Saline + 2% |
| solution/ | | human albumin | human albumin | human albumin |
| formulation | | | | |

### Example 6. In Vivo Efficacy

A first in vivo study was performed with 21 female and 4 male NOD SCID gamma (NSG) mice, inoculated with 1×10⁶ melanoma A375 cells/mouse. The tumor-bearing mice were divided into three experimental groups on day 7 post inoculation, and infused (adoptively transferred) with the tested cell composition or control, as follows:

Group 1 (n=8) was infused with 2×10⁷ eTCR-expressing cells (composition 1 in Table 8 above). Group 2 (n=7) was infused with 2×10⁷ negative control cells (transduced with supernatants of PG13 that do not contain retroviruses, also referred to as “no vector”). Group 3 (n=6) mice were infused with saline. On day 15 post adoptive transfer (ACT), blood and spleen samples were analyzed for the presence of transduced cells, and tumor volume and weight were subsequently evaluated, as detailed below.

For evaluating the presence of eTCR-expressing cells in blood and spleen samples, leukocytes obtained from the samples were stained for CD3 and V0313 and analyzed by flow cytometry essentially as described above. Briefly, the spleen lymphocyte samples were prepared by mashing spleens in 20 mL PBS, centrifugation for 6 minutes at 1000 rpm and treating the resulting pellets with 5 mL of Ammonium chloride potassium (ACK) for 5 minutes. For the preparation of blood samples, 100 μl of whole blood is treated with ACK as described above. The resulting cells are washed and 300,000 cells from each sample (spleen and blood) are used for flow cytometry analysis. To this end, cells were seeded in 96-well plates, centrifuged, stained with an antibody mix containing Vβ13- and CD3-specific labeled antibodies and re-suspended in FACS buffer. The results are presented in Table 9 as percentile of CD3 and Vβ13 positive cells out of the total cells processed.

TABLE 9
eTCR expression in mouse blood and
spleen 15 days following ACT

| Animal | % CD3⁺Vβ13⁺ cells in blood | | % CD3⁺Vβ13⁺ cells in spleen | |
|---|---|---|---|---|
| identifier | No vector | eTCR vector | No vector | eTCR vector |
| 1 | 2.17 | 53.19 | 3.12 | 48.56 |
| 2 | 3.52 | 42.42 | 19.21 | 55.14 |
| 3 | 2.38 | 51.11 | 7.46 | 93.44 |
| 4 | 5.13 | 45.83 | 4.80 | 56.40 |
| 5 | 5.04 | 30.00 | 10.96 | 40.40 |
| 6 | 2.83 | 49.09 | 3.21 | 92.31 |
| 7 | 8.16 | 44.90 | 8.13 | 64.38 |
| 8 | | 31.11 | 6.19 | 22.8 |
| Average | 4.18 | 43.46 | 8.13 | 64.38 |
| SDV | 2.12 | 8.68 | 6.19 | 22.8 |

As can be seen in Table 9, eTCR⁺ cells showed remarkable persistence in mouse blood and in spleen, maintaining a high level of eTCR expression with an average of 43% in blood and 48% in spleen as measured 15 days following the onset of ACT.

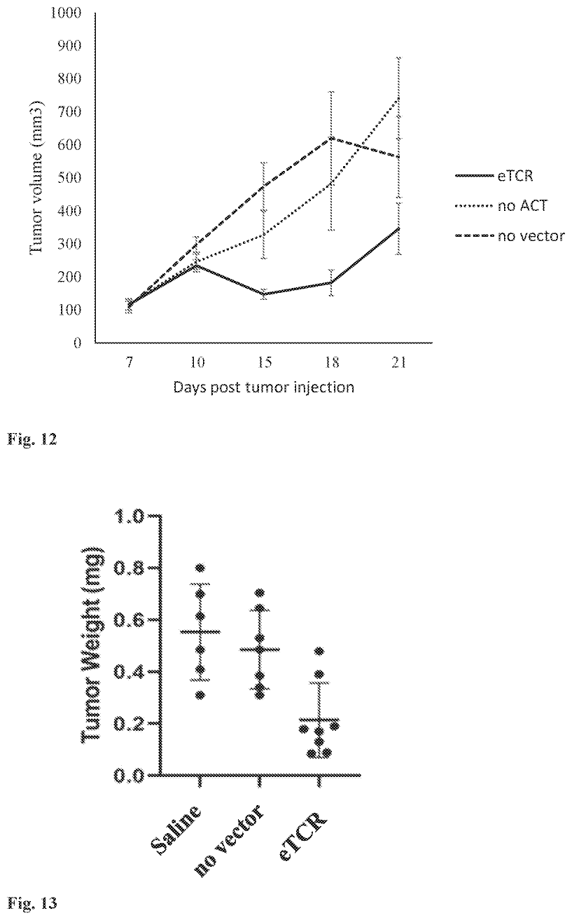

To evaluate the effect of the treatment on tumor development, mice were sacrificed on day 22 post tumor inoculation and the tumors were collected and examined. Tumor volume measurements are presented in FIG. 12, in which A375 tumor volumes determined in cohorts of mice treated with cells transduced with eTCR vector (black line, n=8), cells transduced with negative control PG13 supermatants (no vector, dashed line n=7), or with saline (no ACT dotted line, n=6) are presented. Tumor weights are presented in FIG. 13, indicating the mean±SD.

As can be seen in FIGS. 12 and 13, marked reduction in both the volumes and weights of the tumors was observed with mice treated by the eTCR⁺ cells compared to the two control groups. In particular, FIG. 12 shows a highly significant difference (**p<0.001) in tumor volumes between the eTCR group compared to the saline-treated group, as well as a significant difference (*p<0.01) between the eTCR group and the group treated with vector-free cells. FIG. 13 shows significantly reduced tumor weights (** p<0.01) between the two control groups and the eTCR groups (all calculated using a paired Student's t-test).

In summary, the results demonstrate a potent tumor-specific response induced by an ACT composition of the invention, characterized by prolonged persistence of the adoptively transferred cells and significant inhibition of tumor progression.

### Example 7. Characterization of In Vivo Response

A second in vivo experiment was conducted to evaluate the compositions prepared using clinical-grade viral constructs, in which A375 melanoma cells carrying a luciferase reporter gene were monitored, following administration of different doses of the eTCR⁺ or vector-free cells, as detailed in Table 10 below.

To this end, A375 cells were genetically modified to express luciferase, as a reporter protein, and a puromycin resistance gene. To select for luciferase expressing cells, transfected cells were cultured in the presence of 5 μg/mL puromycin for two weeks prior to tumor cells inoculation. The resulting cells emit light following exposure to the luciferase substrate luciferin, and accordingly mice were injected with luciferin following treatment to facilitate imaging of the tumors in vivo. The luciferin (VivoGlo) was administered IV 5 minutes before imaging.

Escalating doses of the cells formulated in saline +2% human albumin, were administrated intraperitoneally (IP) to male NSG mice. Due to the variation in tumor development in the individual mice, the treatment groups were constructed such that the average tumor volume in all groups would be similar.

TABLE 10
Treatment and dosage administration regime

| | Mice per | | | Adoptively | |
|---|---|---|---|---|---|
| Group | group | Treatment | Tumor injected | transferred cells | Imaging |
| 1 | 8 | No Treatment (NT) | A375-luc | None | Mice |
| 2 | 7 | No vector | 1 × 10⁶/mouse | 28 × 10⁶ Control | injected with |
| 3 | 6 | eTCR | | 14 × 10⁶ eTCR⁺ | VivoGlo |
| 4 | 7 | eTCR | | 21 × 10⁶ eTCR⁺ | luciferase |
| 5 | 9 | eTCR | | 28 × 10⁶ eTCR⁺ | substrate |
| 6 | 6 | eTCR | | 56 × 10⁶ eTCR⁺ * | |

* Group 6 - dosage was divided into two injections administered one week apart.

At day 6 post ACT, three of the treated mice from each group were sacrificed. Tumors were weighed and fixed in buffered-formalin and analyzed by immunohistochemistry (IHC) for the human marker CD3. Organs (heart, lungs, kidneys, spleen, brain and liver) were preserved in formalin or flash frozen and stored at −80° C. to enable examination for the presence of adoptively transferred cells by IHC and qRT-PCR, respectively. Various parameters including anti-tumor efficacy, persistence of eTCR⁺ cells in the blood of the treated mice; tumor infiltration by the eTCR⁺ cells close to the time of administration (day 6 post ACT); and potential for tumorigenicity during a 4-months follow-up period were determined, as described below.

### A. Anti-Tumor Efficacy

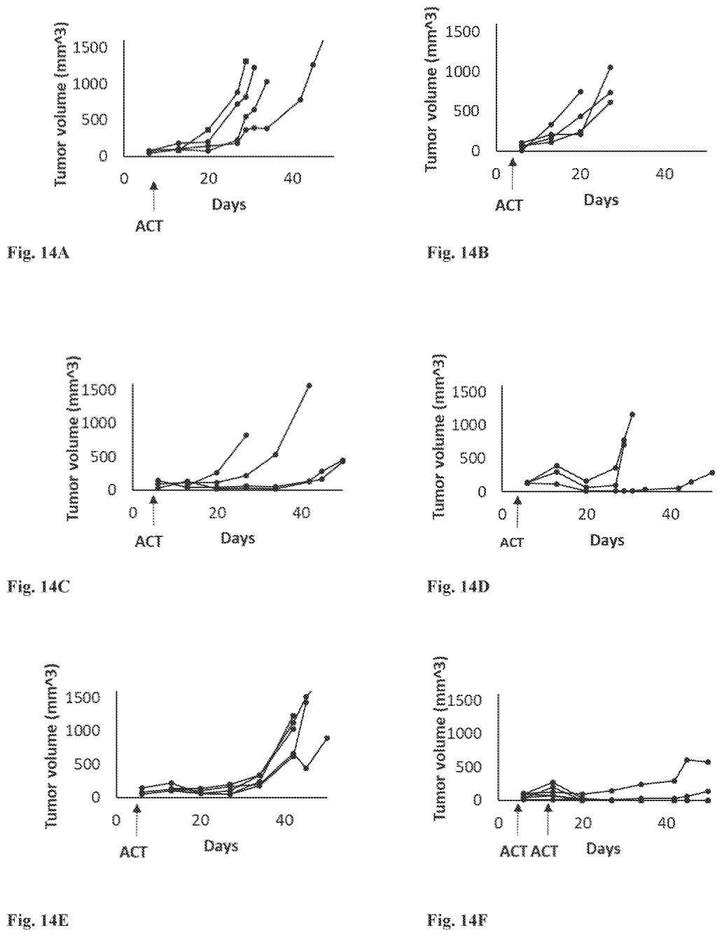

FIGS. 14A-14F present plots of tumor volumes (calculated based on caliper measurements) in individual mice according to tested groups 1-6 as detailed in Table 10, respectively. The results demonstrated that an eTCR⁺ ACT composition produced in a controlled manner with GMP-grade virus supernatant, is an effective treatment in reducing tumor volume in this NSG melanoma xenograft model. eTCR⁺ compositions succeeded at reducing tumor load even at the lowest dose examined, in correlation with the absolute number of tumor cells administered, with complete inhibition of tumor development measured at a dose of 56×10⁶ eTCR⁺ per mouse.

The average survival over time was calculated for each of the tested groups. FIG. 15 presents the Kaplan Meier survival curves for each group (p<0.01): no treatment appears as tumor only—line with full rhombus, negative control (no vector cells)—line with vertical dashes on the top, eTCR⁺ cells: 14×10⁶—line with full squares, 21×10⁶—line with full hexagon, 28×10⁶—line with full circle, 56×10⁶ line with empty triangle.

The results reveal a clear dose dependent anti-tumor activity, as a delay in tumor growth and overall survival rates was observed in all eTCR⁺ ACT treated mice compared to the no vector or non-treated mice groups. Notably, the groups treated with 28×10⁶ eTCR cells and 56×10⁶ eTCR cells show an outstanding 100% survival rate throughout the entire experiment period of about 50 days.

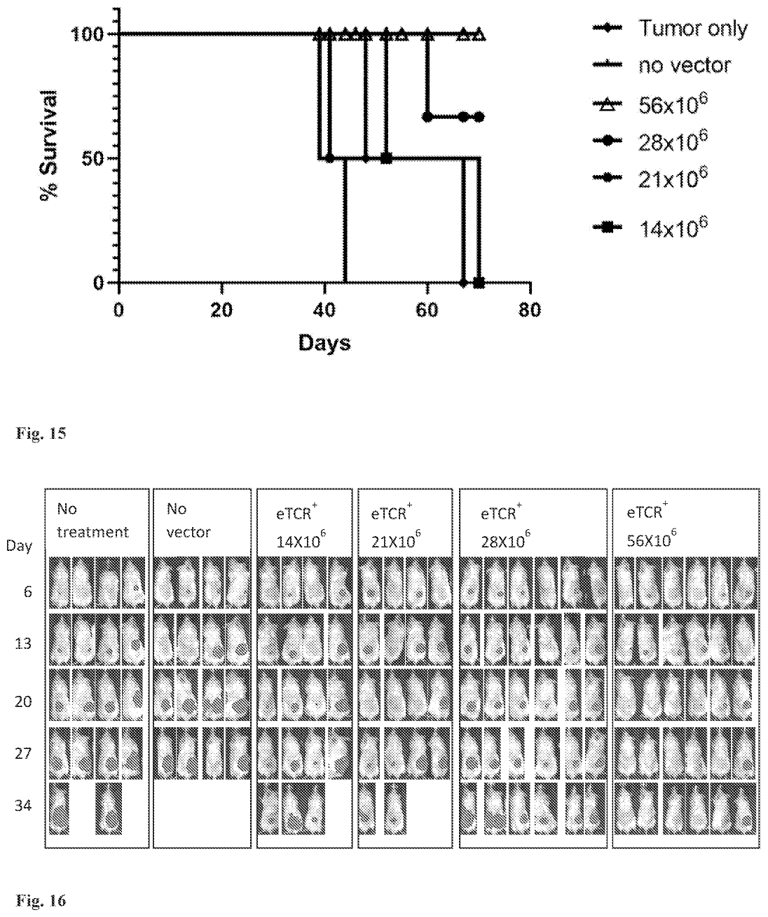

Bioluminescence of the luciferase-expressing A375 melanoma cells was monitored until day 34 post inoculation, and results are presented in FIG. 16 (for days 6, 13, 20, 27 and 34). As can be seen, the groups treated with eTCR⁺ cells showed reduced or delayed tumor growth compared to controls, which appeared to be dose-dependent. It is noted that tumor growth in the majority of mice in the non-treated mice group reached the volume in which mice had to be sacrificed prior to day 34, for ethical reasons.

B. Biodistribution. Persistence and Characterization of Adoptively Transferred Cells

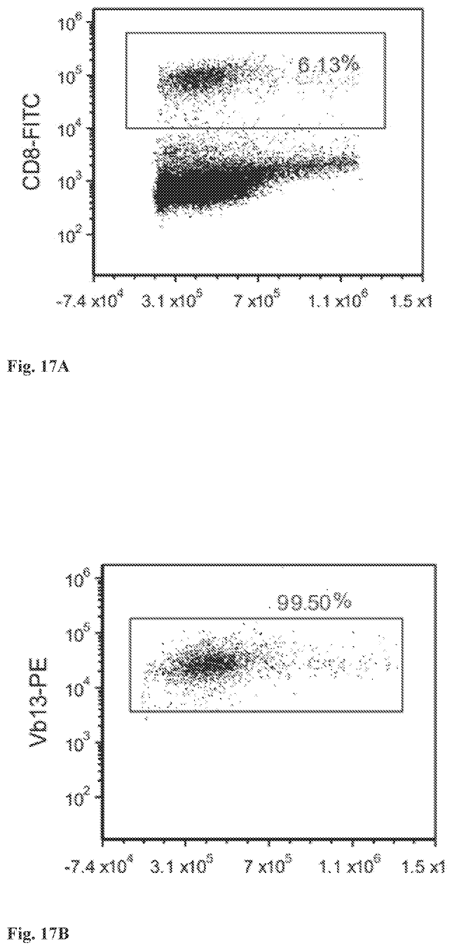

The persistence of eTCR⁺ cells in blood of NSG tumor-bearing mice was assessed at different time points post ACT. To this end, blood samples obtained from mice at days 6, 19 and 33 were stained with FITC-conjugated anti human CD8. The CD8⁺ cell population was further stained with PE-conjugated anti-Vβ13 antibodies, and analyzed by flow cytometry. FIGS. 17A-17B show the results in mice receiving 56×10⁶ eTCR⁺ cells at day 33 following the first infusion stained for CD8 and Vβ13, respectively.

As can be seen, all CD8⁺ cells circulating in the blood of mice infused with 56×10⁶ eTCR⁺ cells (which achieved complete anti-tumor response) were positive for the eTCR at day 33, demonstrating a remarkable and significant persistence of the adoptively transferred cells. Further, these findings indicate an in vivo clonal proliferation of the eTCR⁺ cells, as the starting transduction percentage under the experimental conditions employed was 45%. Surprisingly, in mice that received the maximal dose of eTCR⁺ cells, expression of the activation marker 4-1BB was further detected in human lymphocytes circulating in the peripheral blood of the mice.

Finally, characterization of T cell populations in peripheral blood of the mice post ACT was conducted. Blood from the different treatment groups was sampled at days 6, 19 and 33 post ACT. Human CD4⁺ (hCD4⁺) or Human CD8⁺ (hCD8⁺) cells were detected using the corresponding antibodies and analyzed by FACS. Results are presented in Table 11, in which “2ACT” indicates that the cells were divided to two 28×180 doses and administered one week apart.

TABLE 11
Characterization of T cells in peripheral blood of mice post ACT

| | | % Human T cells in | | |
|---|---|---|---|---|
| | | peripheral blood | | |
| | Group by | Day | Day | Day |
| Marker | treatment | 6 | 19 | 33 |

% hCD8⁺

| | | | |
|---|---|---|---|
| No vector | 2 | 0.88 | NA |
| 14 × 10⁶ | 1.08 | 0.44 | 0.42 |
| 21 × 10⁶ | 1.15 | 0.83 | 0.445 |
| 28 × 10⁶ | 0.95 | 1.3 | 0.46 |
| 56 × 10⁶ (2ACT) | 0.95 | 1.89 | 5.25 |

% hCD4⁺

| | | | |
|---|---|---|---|
| No vector | 2 | 1.89 | NA |
| 14 × 10⁶ | 1.75 | 0.75 | 0.68 |
| 21 × 10⁶ | 2.97 | 1.07 | 0.49 |
| 28 × 10⁶ | 2.54 | 2.98 | 0.82 |
| 56 × 10⁶ (2ACT) | 2.54 | 10.65 | 15.18 |

Results shows that at days 19 and 33, the proportion of human T cells in the peripheral blood of the eTCR⁺ ACT treated mice was markedly and continuously increased over time, attesting to active expansion of the transfused cells.

In summary, the results of this experiment demonstrate remarkable and dose-dependent efficacy of the clinical-grade eTCR⁺ ACT compositions, accompanied by prolonged persistence and in-vivo clonal expansion over time. The results further show adequate safety and significantly extended survival of the treated animals compared to controls.

### Examples 8. In Vivo Evaluation of Cell Compositions Produced by Different Manufacturing Protocols

Clinical-grade cell compositions produced by different protocols as described in Example 5 were examined in the xenograft in vivo model essentially as described in Examples 6 and 7. Briefly, female NSG mice were implanted with luciferase-expressing A375 melanoma cells and infused with the various ACT compositions as summarized in Table 12 below.

TABLE 12
Treatment regime with cells produced using different expansion protocols

| | Mice | | | |
|---|---|---|---|---|
| | per | | Tumor | Number of eTCR⁺ T cells in |
| Group | group | ACT treatment/protocol | cells | ACT |
| 1 | 6 | No treatment (tumor only) | A375-luc | — |
| 2 | 3 | No vector | 1 × 10⁶ per | — |
| 3 | 6 | eTCR⁺ cells prepared | mouse | 13 × 10⁶ positive cells/ |
| | | using anti-CD3 + feeders | | 28 × 10⁶ total |
| 4 | 3 | eTCR⁺ cells prepared | | 13 × 10⁶ positive cells/ |
| | | using Transact | | 18.6 × 10⁶ total |
| 5 | 5 | eTCR⁺ cells prepared | | 13 × 10⁶ positive cells/ |
| | | using Dynabeads | | 17.3 × 10⁶ total |

More specifically, the manufacture processes are described in greater detail in Table 4 above, and the specific cell composition administered to group 3 corresponds to Composition 3 of Table 8.

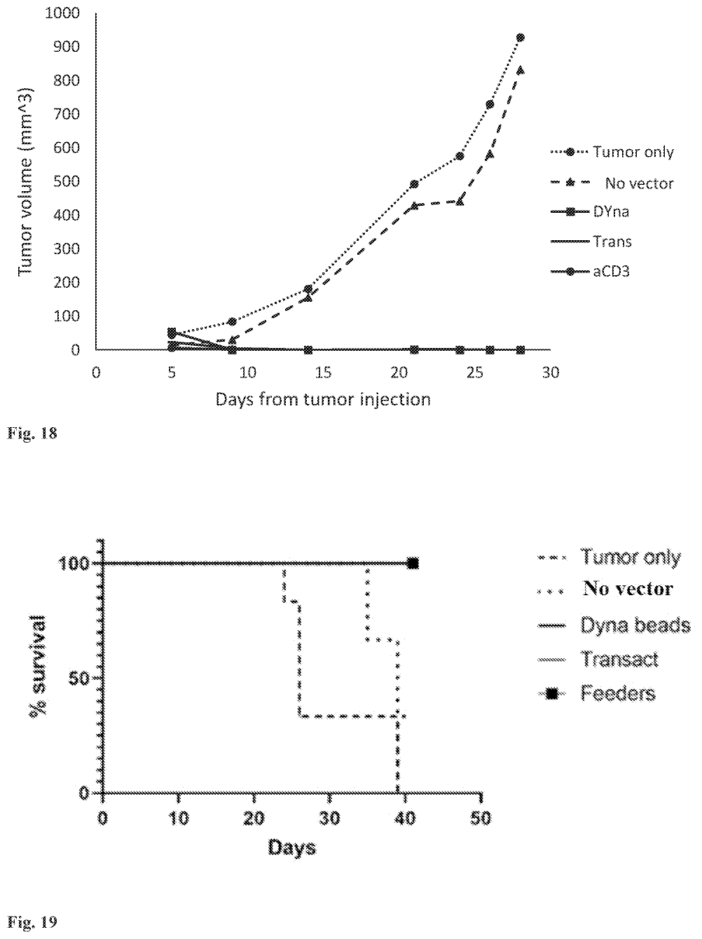

FIGS. 18 and 19 depict the results of tumor volume and survival in the different groups, respectively. In FIG. 18, no treatment appears as tumor only—dotted line with full circle, no vector—dashed line with full triangle, Dyna beads appears as DYna—black line with full squares, Transact appears as Trans—black line and anti-CD3—aCD3, black line with full circles. In FIG. 19, Kaplan-Meier curves are presented in which no treatment appears as tumor only—dashed line, no vector—dotted line, Dynabeads appears as DYna—thick line, Transact appears as Trans—thin line, anti-CD3 appears as Feeders—squares.

As can be seen, all production protocols yielded ACT compositions effective in eradicating tumor growth. Further, all treated mice receiving eTCR⁺ ACT survived throughout the monitoring period, while all mice in the control groups were dead at day 40.

Finally, GMP-compatible protocols for use in the clinical trial were further evaluated using healthy donor PBMC, transduced and expanded according to the “anti-CD3+feeders” protocol in a GMP-grade facility. The results, presented in Table 13 below, show remarkable transduction efficacies in the preparation of allogeneic ACT compositions, with an average of about 63% after 13 days of culture, which were even higher and more consistent at day 15, approaching a transduction rate of about 70% (with an average greater than 69%). Thus, a 15-day expansion period was used in the clinical trial using autologous ACT compositions described below.

TABLE 13
Transduction rates in clinical trial facility

| | Day 6* | | | Day 13* | | | Day 15** | | |
|---|---|---|---|---|---|---|---|---|---|
| Test | 1 | 2 | 3 | 1 | 2 | 3 | 1 | 2 | 3 |
| eTCR⁺ | 47.60 ± | 70.96 ± | 67.8 ± | 52.52 ± | 60.2 ± | 75.34 ± | 69.64 | 69.95 | 68.44 |
| cells | 6.09 | 1.30 | 1.55 | 7.62 | 0.52 | 0.08 | | | |
| (%) | | | | | | | | | |

*Average of two samples
**1 bag with the product dose was prepared.

### Example 9. Clinical Trial

A clinical trial was conducted in human patients with NY-ESO-1-expressing metastatic cancers, in which autologous PBMC were obtained from a patient, subjected to ex-vivo modifications to obtain an ACT composition as disclosed herein, and subsequently infused back into the patient. The ACT composition and viral source material are manufactured and formulated according to GMP-compatible protocols essentially as described in the aforementioned Examples, and are described in greater detail below.

Briefly, the manufacture procedure of the ACT composition included the following major steps, wherein the overall length of the production procedure (following collection of source material) was approximately 14-21 days. These steps are described in greater detail below.
1. Collection of source material (leukapheresis);
2. Purification of the PBMC by density gradient;
3. Ex vivo stimulation;
4. Transduction of stimulated cells with retroviral vector;
5. Expansion of the transduced cells;
6. Harvesting of the cells and preparation of the cell composition; and
7. Preparation of the final formulation for infusion (dosage form).

Screening of patients (aged between 18 and 70 years) for NY-ESO-1 expression was conducted by immunohistochemistry of resected tissue, and patient expressing tumor related NY-ESO-1 epitope were determined to be eligible for treatment. Screened patients were afflicted with solid tumors, and additional inclusion criteria were inter alia metastatic cancer or locally advanced refractory/recurrent malignancy not amenable to curative treatment, and HLA-A*0201 or A*0206 expression.

The eligible patients underwent leukapheresis such that approximately two times the patient's total blood volume was processed and at least 2.5×10⁹ mononuclear cells were collected. Following leukapheresis, the collected material was tested for cell number and viability, number and percentage of CD3⁺ cells, sterility and mycoplasma. PBMC collected from the pheresis by density gradient then underwent activation (also referred to herein as stimulation) using 50 ng/mL soluble anti-CD3 antibody (OKT3) in the presence of 300 IU/mL recombinant IL-2 for two days. Optionally, collected PBMC were subjected to cryopreservation and thawing prior to stimulation.

Next, the cells resulting from PBMC activation were transduced by a clinical-grade PG13 supernatant comprising a retrovirus encoding the eTCR essentially as described in Example 1, using retronectin. Briefly, the wells of 6-well plates were coated with 25 μg/mL Retronectin in 1.5 mL PBS and blocked with 2.5% human albumin in PBS. Retronectin-coated wells were then coated with PG13 supernatants diluted in culture medium without IL-2 by spinoculation (including centrifugation at 2000G for 2 hours at 23° C.). Cells resulting from the 2-days activation were washed in culture medium and seeded at 0.5×10⁶ cells/mL in CM with 300 IU/mL IL-2. Centrifugation was performed at 1000G for 10 min at room temperature. Plates were incubated overnight in a 37° C. 5% CO₂ humidity incubator.

Transduced cells underwent expansion by culturing with irradiated allogeneic PBMC, used as feeder cells. To this end, allogeneic PBMC were collected under informed consent procedure, kept frozen until use in the manufacturing process, and were irradiated at 7000 RAD prior to their utilization. Next, transduced cells and feeder cells were incubated in AIMV medium with IL-2 (3000 IU/mL) at a ratio of 1:50 (transduced cells to feeder cells). Following the rapid expansion step, a clinical dose of 1×10⁹ to 1×10¹⁰ of transduced expanded cells were obtained.

For the preparation of the final GMP cell composition for infusion, the expanded cells were harvested and suspended in 300 ml of 0.9% saline with 2% human albumin and 300 IU/mL IL-2, to obtain a formulation amenable for intravenous (IV) administration to the patients. The resulting ACT composition was then packaged in the infusion bag (Macopharma, 600 ml VSE 4001XA), labelled with patient identifiers, relevant product information, expiry period, warnings and precautionary statements including “For autologous use only”, according to local regulatory requirements.

The process was performed according to a series of Standard Operating Procedures (SOPs) by specifically trained personnel. A quality management system (QMS) was in place which monitors all aspects of the manufacture, including raw materials, intermediate products and final product (receipt, inspection, testing, handling, labelling, traceability storage and inventory); facility and equipment (maintenance and calibration); personnel training and responsibilities; batch manufacture SOPs; batch records; packaging, transport, stability and shelf life.

A series of quality control (QC) tests assessing viability, cell number, identity, purity, % of transduction, potency, replicate competent virus (RCR), mycoplasma and sterility were performed throughout the various stages of the manufacturing process. The preliminary results of the sterility, potency and percent of transduced cells performed at day 6 of manufacturing were available prior to starting of the lymphodepletion regimen (day 10).

Prior to administration of the cell composition to the patient, a non-myoablative lymphodepletion procedure was commenced 5 or 6 days prior to ACT administration. Patients were treated according to one of the following two protocols for lymphodepletion according to their clinical status and history of bone marrow recovery, on days 5, 4, and 3, pre administration:
1. Cyclophosphamide (CYTOXAN) 250 mg/m² and Fludarabine 25 mg/m².
2. Cyclophosphamide 250 mg/m².

The cell composition was then infused at doses ranging from 1×10⁹ cells to 1×10¹⁰ cells, each elected predetermined dose was administered to a single patient. This dose escalation protocol was employed, to determine the maximal tolerated dose. IL-2 may be continuously infused (e.g. 18×10⁶ IU/24h administrated 24 hours post ACT infusion and for 5 days or until a dose limiting toxicity is observed).

Safety and tumor progression were monitored (e.g. by tumor detecting imaging and scanning methods and blood characteristics analysis). In addition, the presence and persistence of transduced cells in the patients' blood was evaluated.

Summarized in Table 14 below are the details of five treated patients.

TABLE 14
Patient details

| Patient | Tumor type | Prior treatment | ACT dose |
|---|---|---|---|
| 1 | Uveal Melanoma | Immunotherapy | 1 × 10⁹ |
| 2 | Synovial Sarcoma | Chemotherapy | 1 × 10⁹ |
| 3 | Triple-Negative Breast | Chemotherapy | 1 × 10⁹ |
| | Cancer (TNBC) | | |
| 4 | Uveal Melanoma | Immunotherapy | 5 × 10⁹ |
| 5 | Ovarian Carcinoma | Chemotherapy | 5 × 10⁹ |

Evaluation of the resulting ACT compositions indicated the presence of both CD4⁺ and CD8⁺ eTCR⁺ cells, with a ratio of CD8⁺ cells to CD4⁺ cells ranging from about 1.87 to about 12.5 of the eTCR⁺ cells. In addition, transduction efficacy could be maintained at an average of about 53% even when using PBMC from immune compromised or otherwise impaired patients as detailed above (a rate significantly higher than the regulatory requirement of >10%), and could be enhanced up to about 82% by increasing the construct copy number per transduced cell (CPN).

The functionality and potency of the ACT composition were evaluated prior to treatment, by measuring their IFN-γ secretion upon target recognition essentially as detailed in Example 3. Briefly, the activated transduced or non-transduced PBMCs were co-cultured with T2 cells, either loaded with NY-ESO-1 peptide or unloaded, at a 1:1 effector to target (E:T) ratio for 24 hours. Following culture, supernatants were examined for IFN-γ secretion by ELISA (at day 6 post-activation). Exemplary results for ACT compositions prepared for patient 1 (uveal melanoma, previously treated by a combination of PD-1 and CTLA4 checkpoint inhibitors) are presented in FIG. 21, in which non-specific T2 cells incubated with unloaded T2 cells are denoted “T:T2”, non-specific T cells incubated with peptide-loaded T2 cells are denoted “T:T2-ESO”, eTCR⁺ T cells incubated with T2 cells are denoted “eTCR⁺:T2”, and eTCR⁺ T cells incubated with peptide-loaded T2 cells are denoted “eTCR⁺:T2-ESO”.

As can be seen in FIG. 21, eTCR⁺ ACT compositions secreted high levels of IFN-γ upon specific recognition of the NY-ESO-1 peptide, wherein no detectable secretion was observed in any of the control groups. Further, the IFN-γ levels measured for ACT compositions prepared from autologous PBMC of cancer patients, previously treated with chemotherapy or immune modulating treatments, were found to be comparable to those measured for ACT compositions prepared from PBMC of healthy donors using the constructs of the invention, indicating potent anti-tumor activity as described in Example 2 (and improvement over hitherto reported compositions), of autologous ACT modalities under clinical settings.

Further, the ACT compositions were very well tolerated and exhibited remarkable safety profiles, with no severe adverse events related to the transferred cells.

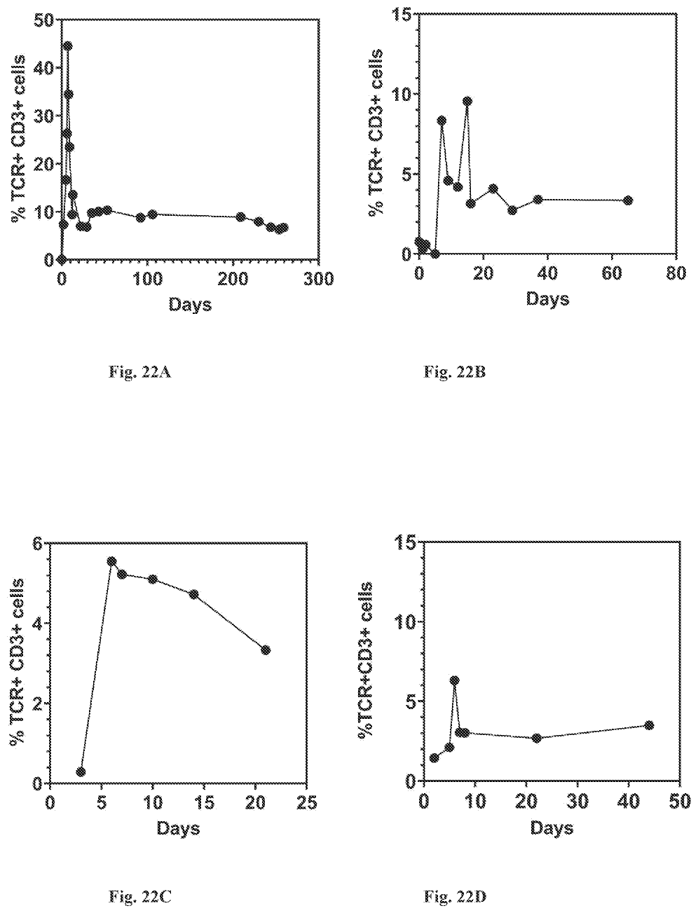

The persistence of eTCR⁺ cells in blood of the treated patients was monitored at different time points following ACT. To this end, PBMCs obtained from each of the tested patients were stained with FITC-conjugated anti human CD3. The CD3⁺ cell population was further stained with PE-conjugated anti-Vβ13 antibodies, and analyzed by flow cytometry. FIGS. 22A-22D show the percent of eTCR⁺ expressing CD3⁺ in PBMC of patients 1 to 4, respectively, receiving an ACT dosage of 1×10⁹ or 5×10⁹ eTCR⁺ cells, as indicated in Table 14.

As can be seen in FIGS. 22A-22D, high levels of eTCR⁺ expressing CD3⁺ cells were detected in all patients following ACT, which gradually stabilized to remain in the range of 3-10% of the PBMCs. Remarkably, eTCR⁺ expressing CD3⁺ cells could be detected in peripheral blood for prolonged periods, and even at the latest time point monitored for each of the patients, namely up to 9 months of the onset of ACT (FIG. 22A). The results indicate that that the transduced cells adopted a highly persistent phenotype.

In summary, the results demonstrate that ACT compositions could be prepared not only from the blood of healthy donors, but also from PBMC of patients with refractory solid tumors, which had failed to respond to treatment with chemotherapy or immunotherapy. These patients are often immunocompromised due to the presence of the tumor and/or prior treatment, but were nevertheless amenable for the preparation of ACT compositions in accordance with the invention. The results further demonstrate remarkable in vivo safety and persistence in peripheral blood of the resulting ACT compositions; as opposed to many hitherto reported compositions in which persistence was demonstrated for up to 30 days, compositions in accordance with the invention could even be detected in peripheral blood of patients for up to 1.5-9 months. Finally, in vitro evaluation of the functional capacity of the compositions suggests the ability to exert improved anti-tumor activity.

### Example 10. Characterization of eTCR⁺ CD4⁺ Cells

The functionality of eTCR⁺ CD4⁺ cells (CD8-independent activity) was further evaluated and characterized as detailed below. CD4⁺ cells were isolated by magnetic beads (Milteny 130-096-533) from PBMC of healthy donors. The resulting cells were activated with anti-CD3 and anti-CD28 antibodies (OKT3 and CD28.2 clones, from Invitrogen and Biologend, respectively, each at a concentration of 1 μg/ml) two days prior to transduction with the eTCR viral vector. Following transductions, cells were maintained in culture medium containing 300 IU/ml IL-2 for 9 days until subjected to the target-induced cytokine secretion assay.

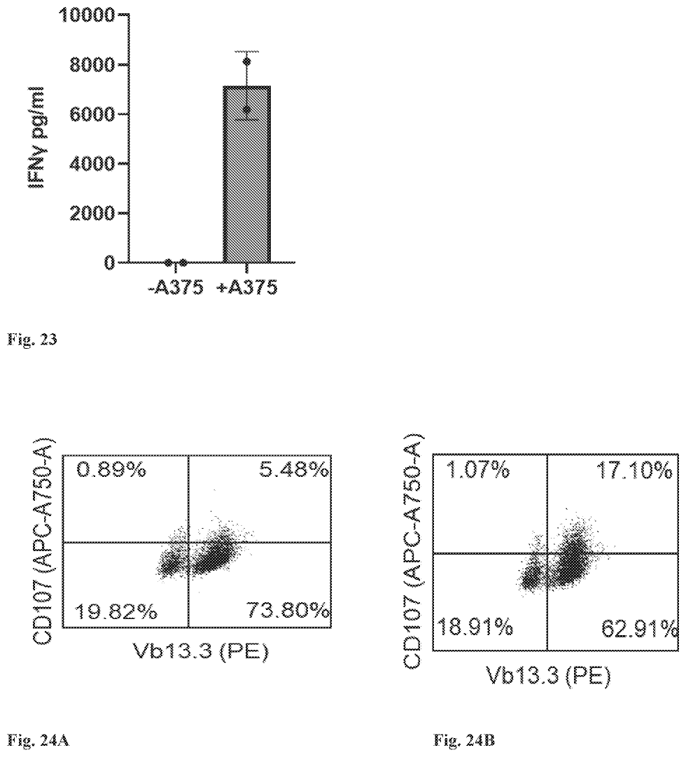

IFN-γ secretion was evaluated as described in Example 3. Briefly, transduced cells were cultured with or without A375 melanoma target tumor cells (expressing the NY-ESO-1 target antigen). Incubation was performed at an effector:tumor cell ratio of 1:1 (100,000 cells from each population) for 24 hours. Following incubation, the levels of IFN-γ secreted to the culture supernatant were measured by ELISA. Results are presented in FIG. 23, in which cells incubated with A375 melanoma target tumor are denoted “+A375” and control cells incubated in the absence of target cells are denoted “-A375”.

The results show that eTCR⁺ CD4⁺ cells were able to secrete high levels of IFN-γ in the presence of NY-ESO-1-expressing target tumor cells, whereas no detectable IFN-γ levels were measured in the absence of the tumor cells. These results indicate that the eTCR is not merely detectable on the surface of transduced CD4⁺ T cells (eTCR⁺ CD4⁺) in ACT compositions prepared in accordance with the invention, but rather that the constructs and protocols employed enabled the eTCR to be expressed in a functional manner.

Next, CD4⁺ cells transduced as discussed above were further evaluated phenotypically, by examining their expression of CD107. CD107 (Lysosomal Associated Membrane Protein-1, LAMP-1) is a cell surface protein, involved in the degranulation process of activated CD8⁺ T cells and NK cells. Cells were incubated for 6 hours of in the presence or absence of the A375 target cells, as detailed above. Following incubation, CD107, CD4 and the eTCR β chain (Vb13.3) were stained with differentially labeled antibodies, and measured by FACS. Results following the incubation in the absence or presence of the A375 melanoma cells are presented in FIGS. 24A and 24B, respectively.

As can be seen in FIG. 24A, eTCR⁺ CD4⁺ cells surprisingly exhibited significant levels of CD107 expression even in the absence of the target cells (5.48%), whereas less than 1% of the gated eTCR⁻ cells exhibited CD107 surface expression. Further, as can be seen in FIG. 24B, CD107 surface expression in eTCR⁺ CD4⁺ cells was further enhanced by about threefold in the presence of the NY-ESO-1-expressing target cells, reaching 17.1%, indicating a target-specific enhancement of the cytotoxic effector marker CD107. Similar results were also obtained when the experiments were performed when healthy donor PBMC (without pre-separation of CD4⁺ cells) were transduced and co cultured with tumor cells, wherein evaluation of CD107 levels specifically on CD4⁺ cells was performed by gating said cells during flow cytometry.

CD107 is considered a T cell activation and cytotoxicity marker, wherein its expression level serves as an indicator for T cell activation and effector functions (such as degranulation) in cytotoxic lymphocytes. CD107 is not normally expressed on helper CD4⁺ cells (such as Th1 and Th2 cells), and was only detected in certain T cell populations adopting a CD4⁺ CTL phenotype upon activation. Accordingly, without being bound by a theory or mechanism of action, the results indicate that the eTCR⁺ CD4⁺ cells adopted a cytotoxic effector phenotype, that does not normally characterize CD8⁻ cells such as helper (CD4⁺) T cells. It was further found that the incidence of the CD8⁻ effector cell population (CD4⁺ eTCR⁺ CD107⁺ cells, consistent with the CD4⁺ CTL phenotype) out of the total CD4⁺ eTCR⁺ was about 15%.

In summary, the results show that the constructs and protocols of the invention provide for the production of cell compositions comprising substantial amounts of both CD8⁺ and CD4⁺ eTCR⁺ cell populations, and that the CD4⁺ eTCR⁺ cells are capable of exerting a CD8-independent NY-ESO-1-specific activity (which can also be regarded as being MHC II-non-obligatory). Further, the results show that the transduced CD4⁺ eTCR⁺ cells adopt a unique phenotype characterized by the surface expression of CD107, thus demonstrating the potential to enhance the overall cytotoxic activity of the ACT compositions.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. An isolated T cell receptor (TCR) directed to NY-ESO-1, comprising:
(a) a TCR α chain comprising:
i. a variable region (VR) comprising a CDR1 having the amino acid sequence of SEQ ID NO: 1, a CDR2 having the amino acid sequence of SEQ ID NO: 2, and a CDR3 having the amino acid sequence of SEQ ID NO: 3;
ii. a constant region (CR) comprising a cysteine residue at position 47 thereof and the amino acid sequence as set forth in SEQ ID NO: 22 at positions 250-254 thereof;
(b) a TCR β chain comprising:
i. a VR comprising a CDR1 having the amino acid sequence of SEQ ID NO: 4, a CDR2 having the amino acid sequence of SEQ ID NO: 5, and a CDR3 having the amino acid sequence of SEQ ID NO: 6; and
ii. a CR comprising a cysteine residue at position 57 thereof, and
(c) a plurality of interchain disulfide bonds between the α chain and β chain.

2. The isolated TCR of claim 1, comprising a first disulfide bond between the cysteine residues at positions 94 of the α chain CR and 131 of the β chain CR, and a second disulfide bond between the cysteine residues at positions 47 of the α chain CR and 57 of the β chain CR; and/or
wherein the transmembrane region of the α chain has the amino acid sequence LLVIVLRILLLKVAGFNLLMT (SEQ ID NO: 23).

3. (canceled)

4. The isolated TCR of claim 1, comprising a TCR α chain having the amino acid sequence as set forth in SEQ ID NO: 17, optionally excluding the signal peptide at positions 1-20 thereof, and/or comprising a TCR β chain having the amino acid sequence as set forth in SEO ID NO: 18, optionally excluding the signal peptide at positions 1-21 thereof.

5. (canceled)

6. The isolated TCR of claim 1, in which each of the α and β chains exhibits at least 95% sequence identity to SEQ ID NOs: 17 and 18, respectively, and wherein said TCR is capable of specific binding to an HLA-A2-presented epitope comprising the amino acid sequence SLLMWITQC (SEQ ID NO: 21).

7. A nucleic acid construct encoding the isolated TCR of claim 1.

8. The nucleic acid construct of claim 7, comprising the nucleic acid sequences as set forth in SEQ ID NOs: 7-9, encoding the CDR1, CDR2 and CDR3 of the α chain, respectively, and the nucleic acid sequences as set forth in SEQ ID NOs: 10-12, encoding the CDR1, CDR2 and CDR3 of the β chain, respectively.

9. The nucleic acid construct of claim 8, comprising a first nucleic acid molecule encoding the α chain and having the nucleic acid sequence as set forth in SEQ ID NO: 19, and a second nucleic acid molecule encoding the β chain and having the nucleic acid sequence as set forth in SEQ ID NO: 20, or
comprising a first nucleic acid molecule encoding the α chain and having at least 95% identity to the nucleic acid sequence as set forth in SEO ID NO: 19, and a second nucleic acid molecule encoding the β chain and having at least 95% identity to the nucleic acid sequence as set forth in SEO ID NO: 20, wherein the encoded TCR is capable of specific binding to an HLA-A2-presented epitope comprising the amino acid sequence of SEQ ID NO: 21.

10. The nucleic acid construct of claim 7, wherein said first and second nucleic acid molecules are operatively linked to one or more transcription control elements capable of inducing or enhancing the expression of said TCR chains in a human lymphocyte, or wherein the first nucleic acid molecule and the second nucleic acid molecule are connected by a third nucleic acid molecule encoding a linker, wherein the linker is a 2A peptide.

11. (canceled)

12. (canceled)

13. The nucleic acid construct of claim 7, wherein the encoded TCR is capable of exerting tumor-specific reactivity in a CD8-independent manner, or
capable of providing an enhanced level and/or duration of expression of the encoded TCR, as compared to a construct encoding a naturally-occurring TCR directed to an HLA-A2-presented epitope as set forth in SEO ID NO: 21.

14. (canceled)

15. (canceled)

16. (canceled)

17. (canceled)

18. An expression vector comprising the nucleic acid construct of claim 7.

19. A host cell comprising the vector of claim 18.

20. A pharmaceutical composition comprising a therapeutically effective amount of the vector of claim 18.

21. A cell composition for cancer immunotherapy, comprising a therapeutically effective amount of immune cells engineered to express a TCR directed to NY-ESO-1, the TCR comprising:
(a) a TCR α chain comprising:
i. a VR comprising a CDR1 having the amino acid sequence of SEQ ID NO: 1, a CDR2 having the amino acid sequence of SEQ ID NO: 2, and a CDR3 having the amino acid sequence of SEQ ID NO: 3;
ii. a CR comprising a cysteine residue at position 47 thereof and the amino acid sequence as set forth in SEQ ID NO: 22 at positions 250-254 thereof;
(b) a TCR β chain comprising:
i. a VR comprising a CDR1 having the amino acid sequence of SEQ ID NO: 4, a CDR2 having the amino acid sequence of SEQ ID NO: 5, and a CDR3 having the amino acid sequence of SEQ ID NO: 6; and
ii. a CR comprising a cysteine residue at position 57 thereof; and
(c) a plurality of interchain disulfide bonds between said α chain and β chain, wherein the immune cells are a T cell-containing cell population and said composition is adapted for adoptive transfer therapy (ACT).

22. (canceled)

23. The cell composition of claim 21, wherein the TCR comprises a TCR α chain having the amino acid sequence as set forth in SEQ ID NO: 17, optionally excluding the signal peptide at positions 1-20 thereof, and a TCR β chain having the amino acid sequence as set forth in SEQ ID NO: 18, optionally excluding the signal peptide at positions 1-21 thereof, or
wherein the nucleic acid construct comprises a first nucleic acid molecule encoding the α chain and having the nucleic acid sequence as set forth in SEO ID NO: 19, and a second nucleic acid molecule encoding the β chain and having the nucleic acid sequence as set forth in SEO ID NO: 20.

24. (canceled)

25. The cell composition of claim 21, which is prepared by a process comprising:
a. provided a T-cell containing cell population,
b. culturing the cell population in the presence of a CD3-specific antibody and interleukin-2 (IL-2),
c. engineering the cells resulting from step b. to express said TCR,
d. expanding the cells resulting from step c. so as to obtain a therapeutically effective amount of expanded engineered cells, and
e. harvesting the expanded engineered cells resulting from step d.

26. The cell composition of claim 25, wherein the T-cell containing cell population provided in step a. is a peripheral blood mononuclear cells (PBMC) population, or
wherein step b. further comprises culturing said cells with a CD28-specific antibody, and wherein the CD3-specific antibody and the CD28-specific antibody are surface-bound, or wherein step d. is performed by incubating the cells following step c. with IL-2 and optionally feeder cells, and in the absence of supplementation of antibodies.

27. (canceled)

28. (canceled)

29. (canceled)

30. The cell composition of claim 25, wherein step d. is performed so as to obtain at least 10⁹ CD3⁺ eTCR⁺ cells, or wherein step e. is performed within 7-21 days of initiating step b.

31. (canceled)

32. A process for preparing a cell composition for ACT, the process comprising:
a. provided a T-cell containing cell population,
b. culturing the cells in the presence of a CD3-specific antibody and IL-2,
c. engineering the cells resulting from step b. to express a TCR as defined in claim 1,
d. expanding the cells resulting from step c. so as to obtain a therapeutically effective amount of expanded engineered cells, and
e. harvesting the expanded engineered cells resulting from step d.

33. The process of claim 32, comprising:
a. provided a PBMC population,
b. culturing the cell population in the presence of a CD3-specific antibody and IL-2,
c. engineering the cells to express the TCR by incubating said cells with a viral vector comprising a nucleic acid construct encoding said TCR,
d. expanding the cells following step c. in the presence of IL-2 and feeder cells, and in the absence of supplementation of antibodies, so as to obtain at least 10⁹ CD3⁺ cells expressing said TCR, and
e. harvesting the expanded cells within 14-15 days of initiating step b, and wherein step b. comprises administering to the cells an expression vector as defined in claim 32.

34. (canceled)

35. A method of treating cancer or for enhancing anti-tumor immunity in a subject in need thereof, comprising administering to the subject the cell composition of claim 21 or the pharmaceutical composition of claim 20.

36. The method of claim 35, wherein the cells of the composition are autologous to the subject.

37. The method of claim 35, wherein the subject is afflicted with a NY-ESO-1 expressing tumor, and/or wherein said subject is HLA-A2-positive.

38. (canceled)

39. The method of claim 35, wherein the tumor is selected from the group consisting of: melanoma, myeloma, bladder, ovarian, lung, breast, synovial and prostate tumors.

40-59. (canceled)


SEQUENCE LISTING (29 sequences)

SEQ ID NO: 1 - protein, 6 aa, Synthetic construct
DSAIYN

SEQ ID NO: 2 - protein, 7 aa, synthetic construct
IQSSQRE

SEQ ID NO: 3 - protein, 13 aa, synthetic construct
AVRPLYGGSYIPT

SEQ ID NO: 4 - protein, 5 aa, synthetic construct
MNHEY

SEQ ID NO: 5 - protein, 6 aa, synthetic construct
SVGAGI

SEQ ID NO: 6 - protein, 12 aa, synthetic construct
ASSYVGNTGELF

SEQ ID NO: 7 - DNA, 18 bp, synthetic construct
  source 1..18: mol_type = other DNA
gactctgccatctacaac

SEQ ID NO: 8 - DNA, 21 bp, synthetic construct
  source 1..21: mol_type = other DNA
atccagagctcgcagagggag

SEQ ID NO: 9 - DNA, 39 bp, synthetic construct
  source 1..39: mol_type = other DNA
gccgtgcggcctctgtacggaggctcttatatcccaacc

SEQ ID NO: 10 - DNA, 15 bp, synthetic construct
  source 1..15: mol_type = other DNA
atgaatcacgagtac

SEQ ID NO: 11 - DNA, 18 bp, synthetic construct
  source 1..18: mol_type = other DNA
tccgtgggagcaggaatc

SEQ ID NO: 12 - DNA, 36 bp, synthetic construct
  source 1..36: mol_type = other DNA
gcctcctcttatgtgggcaacaccggcgagctgttc

SEQ ID NO: 13 - protein, 115 aa, synthetic construct
KQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPY

SEQ ID NO: 14 - protein, 140 aa, synthetic construct
IQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 15 - protein, 111 aa, synthetic construct
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVL

SEQ ID NO: 16 - protein, 177 aa, synthetic construct
EDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 17 - protein, 275 aa, synthetic construct
MFETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 18 - protein, 309 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 19 - DNA, 825 bp, synthetic construct
  source 1..825: mol_type = other DNA
  sig_peptide 1..60
atgttcgaaactctgctggggctgctgattctgtggctgcagctgcagtgggtgtcatccaaacaggaggtcactcagattcccgctgccctgagcgtgcctgagggcgagaacctggtgctgaattgctccttcaccgactctgccatctacaacctgcagtggtttaggcaggatccaggcaagggcctgaccagcctgctgctgatccagagctcgcagagggagcagacatccggccgcctgaatgcctctctggacaagtctagcggccggagcaccctgtacatcgcagcaagccagccaggcgattccgccacatacctgtgcgccgtgcggcctctgtacggaggctcttatatcccaaccttcggcagaggcacaagcctgatcgtgcacccttacatccagaacccagaccccgccgtgtatcagctgcgggacagcaagtcctctgataagtccgtgtgcctgttcaccgactttgattcccagacaaacgtgagccagagcaaggactctgacgtgtacatcaccgacaagtgcgtgctggatatgagaagcatggactttaagtccaactctgccgtggcctggagcaataagtccgatttcgcctgcgccaacgcgtttaacaatagcatcatccccgaggatacattctttccttccccagagagctcctgtgacgtgaagctggtggagaagagcttcgagacagatacaaacctgaattttcagaacctgctggtcatcgtgctgcggatcctgctgctgaaggtggccggcttcaatctgctgatgaccctgagactgtggtctagc

SEQ ID NO: 20 - DNA, 930 bp, synthetic construct
  source 1..930: mol_type = other DNA
  sig_peptide 1..63
atgtctatcggcctgctgtgctgtgccgccctgagcctgctgtgggcaggaccagtgaacgcaggagtgacccagacacccaagttccaggtgctgaagaccggccagtctatgacactgcagtgcgcccaggacatgaatcacgagtacatgagctggtatcggcaggatcctggcatgggcctgagactgatccactactccgtgggagcaggaatcaccgaccagggagaggtgccaaacggctataacgtgagcaggagcaccacagaggatttcccactgaggctgctgtctgccgcaccttctcagacaagcgtgtacttttgcgcctcctcttatgtgggcaacaccggcgagctgttctttggcgagggctccaggctgacagtgctggaggacctgaataaggtgttcccccctgaggtggccgtgtttgagccctctgaggccgagatcagccacacccagaaggccaccctggtgtgcctggcaaccggcttctttcctgatcacgtggagctgtcctggtgggtgaacggcaaggaggtgcactctggcgtgtgcacagacccacagcccctgaaggagcagccagccctgaatgactcgagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 21 - protein, 9 aa, synthetic construct
SLLMWITQC

SEQ ID NO: 22 - protein, 5 aa, synthetic construct
LVIVL

SEQ ID NO: 23 - protein, 21 aa, synthetic construct
LLVIVLRILLLKVAGFNLLMT

SEQ ID NO: 24 - protein, 84 aa, synthetic construct
AGGGCAAAGCGCAGCGGATCCGGAGCAACAAACTTTAGCCTGCTGAAGCAGGCAGGCGACGTGGAGGAGAATCCAGGACCGCGG

SEQ ID NO: 25 - DNA, 1839 bp, synthetic construct
  source 1..1839: mol_type = other DNA
atgttcgaaactctgctggggctgctgattctgtggctgcagctgcagtgggtgtcatccaaacaggaggtcactcagattcccgctgccctgagcgtgcctgagggcgagaacctggtgctgaattgctccttcaccgactctgccatctacaacctgcagtggtttaggcaggatccaggcaagggcctgaccagcctgctgctgatccagagctcgcagagggagcagacatccggccgcctgaatgcctctctggacaagtctagcggccggagcaccctgtacatcgcagcaagccagccaggcgattccgccacatacctgtgcgccgtgcggcctctgtacggaggctcttatatcccaaccttcggcagaggcacaagcctgatcgtgcacccttacatccagaacccagaccccgccgtgtatcagctgcgggacagcaagtcctctgataagtccgtgtgcctgttcaccgactttgattcccagacaaacgtgagccagagcaaggactctgacgtgtacatcaccgacaagtgcgtgctggatatgagaagcatggactttaagtccaactctgccgtggcctggagcaataagtccgatttcgcctgcgccaacgcgtttaacaatagcatcatccccgaggatacattctttccttccccagagagctcctgtgacgtgaagctggtggagaagagcttcgagacagatacaaacctgaattttcagaacctgctggtcatcgtgctgcggatcctgctgctgaaggtggccggcttcaatctgctgatgaccctgagactgtggtctagcagggcaaagcgcagcggatccggagcaacaaactttagcctgctgaagcaggcaggcgacgtggaggagaatccaggaccgcggatgtctatcggcctgctgtgctgtgccgccctgagcctgctgtgggcaggaccagtgaacgcaggagtgacccagacacccaagttccaggtgctgaagaccggccagtctatgacactgcagtgcgcccaggacatgaatcacgagtacatgagctggtatcggcaggatcctggcatgggcctgagactgatccactactccgtgggagcaggaatcaccgaccagggagaggtgccaaacggctataacgtgagcaggagcaccacagaggatttcccactgaggctgctgtctgccgcaccttctcagacaagcgtgtacttttgcgcctcctcttatgtgggcaacaccggcgagctgttctttggcgagggctccaggctgacagtgctggaggacctgaataaggtgttcccccctgaggtggccgtgtttgagccctctgaggccgagatcagccacacccagaaggccaccctggtgtgcctggcaaccggcttctttcctgatcacgtggagctgtcctggtgggtgaacggcaaggaggtgcactctggcgtgtgcacagacccacagcccctgaaggagcagccagccctgaatgactcgagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 26 - protein, 399 aa, synthetic construct
ATGGAGACCCTCTTGGGCCTGCTTATCCTTTGGCTGCAGCTGCAATGGGTGAGCAGCAAACAGGAGGTGACGCAGATTCCTGCAGCTCTGAGTGTCCCAGAAGGAGAAAACTTGGTTCTCAACTGCAGTTTCACTGATAGCGCTATTTACAACCTCCAGTGGTTTAGGCAGGACCCTGGGAAAGGTCTCACATCTCTGTTGCTTATTCAGTCAAGTCAGAGAGAGCAAACAAGTGGAAGACTTAATGCCTCGCTGGATAAATCATCAGGACGTAGTACTTTATACATTGCAGCTTCTCAGCCTGGTGACTCAGCCACCTACCTCTGTGCTGTGAGGCCCACATCAGGAGGAAGCTACATACCTACATTTGGAAGAGGAACCAGCCTTATTGTTCATCCG

SEQ ID NO: 27 - protein, 396 aa, synthetic construct
ATGAGCATCGGCCTCCTGTGCTGTGCAGCCTTGTCTCTCCTGTGGGCAGGTCCAGTGAATGCTGGTGTCACTCAGACCCCAAAATTCCAGGTCCTGAAGACAGGACAGAGCATGACACTGCAGTGTGCCCAGGATATGAACCATGAATACATGTCCTGGTATCGACAAGACCCAGGCATGGGGCTGAGGCTGATTCATTACTCAGTTGGTGCTGGTATCACTGACCAAGGAGAAGTCCCCAATGGCTACAATGTCTCCAGATCAACCACAGAGGATTTCCCGCTCAGGCTGCTGTCGGCTGCTCCCTCCCAGACATCTGTGTACTTCTGTGCCAGCAGTTACGTCGGGAACACCGGGGAGCTGTTTTTTGGAGAAGGCTCTAGGCTGACCGTACTG

SEQ ID NO: 28 - protein, 19 aa, synthetic construct
ATNFSLLKQAGDVEENPGP

SEQ ID NO: 29 - protein, 28 aa, synthetic construct
RAKRSGSGATNFSLLKQAGDVEENPGPR